(19) United States
(12) Patent Application Publication
(10) Pub. No.: US 2023/0212513 A1
(43) Pub. Date: Jul. 6, 2023

(54) COMPOSITIONS AND METHODS FOR RED BLOOD CELL DIFFERENTIATION

(71) Applicant: The Children's Medical Center Corporation, Boston, MA (US)

(72) Inventors: Ashlee J. Conway, Boston, MA (US); Tolulope O. Rosanwo, Boston, MA (US); Thomas E. Williamson, Boston, MA (US); Trista E. North, Newton Center, MA (US); George Q. Daley, Cambridge, MA (US)

(73) Assignee: The Children's Medical Center Corporation, Boston, MA (US)

(21) Appl. No.: 18/009,192

(22) PCT Filed: Jun. 17, 2021
(86) PCT No.: PCT/US2021/037784

§ 371 Date: Dec. 8, 2022

Related U.S. Application Data
(60) Provisional application No. 63/041,148, filed on Jun. 19, 2020.

(51) Int. Cl. C12N 5/078; A61K 31/573; A61K 31/727; A61K 35/16; A61K 35/545; A61K 38/18; A61K 38/20; A61K 38/28; A61K 38/40

(52) U.S. Cl. CPC C12N 5/0641; A61K 31/573; A61K 31/727; A61K 35/16; A61K 35/545; A61K 38/18; A61K 38/1816; A61K 38/202; A61K 38/28; A61K 38/40; C12N 2500/25; C12N 2501/125; C12N 2501/14; C12N 2501/2303; C12N 2501/39; C12N 2501/91

(57) ABSTRACT

The invention described herein is directed to compositions and methods for inducing red blood cell (RBC) differentiation. Additionally, provided herein are methods of treating a subject in need thereof by administering the induced RBC described herein.

### CROSS-REFERENCE TO RELATED APPLICATIONS

This application claims benefit under 35 U.S.C. § 119(e) of U.S. Provisional Application No. 63/041,148 filed Jun. 19, 2020, the contents of which are incorporated herein by reference in their entirety.

### GOVERNMENT SUPPORT

This invention was made with government support under Grant Nos R01DK098241 and HL134812 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 4, 2021, is named 701039-097860WOPT_SL.txt and is 3,748 bytes in size.

### TECHNICAL FIELD

The technology described herein relates to red blood cell (RBC) differentiation methods.

### BACKGROUND

Donor-derived blood products (e.g., whole blood, platelets, etc.) are in high demand in clinical medicine, whether to combat trauma or to maintain the health of patients who are lacking in key blood components required to sustain life. The risk of infection, type mismatching, clotting, and the variability of donor blood supply warrants the need to find alternative methods to obtain human blood cells. Engineering patient-specific and/or universal donor human red blood cells (RBCs) in a laboratory setting would allow for a more controlled supply to meet the demands of patients. Engineered RBCs can also be used as models of human blood diseases (e.g., Sickle Cell Anemia), and as a platform on which to develop novel drugs and vaccines (e.g., Malaria), without the need to rely on donor blood supply.

### SUMMARY

The technology described herein is directed to methods of red blood cell (RBC) differentiation. Accordingly, one aspect provided herein is a composition for inducing a red blood cell (RBC) comprising: human AB plasma; heparin; insulin; holo-transferrin; a corticosteroid; and polyvinyl alcohol (PVA).

In one embodiment of any aspect, the composition further comprises at least one growth factor.

In one embodiment of any aspect, the at least one growth factor is selected from the group consisting of IL-3, stem cell factor (SCF), and erythropoietin (EPO).

In one embodiment of any aspect, the corticosteroid is dexamethasone.

In one embodiment of any aspect, the composition further comprises Iscove's DMEM (IMDM).

In one embodiment of any aspect, the composition further comprises at least one antibiotic. In one embodiment of any aspect, the at least one antibiotic is Penicillin and Streptomycin.

In one embodiment of any aspect, PVA is present at a 0.08% concentration.

In one embodiment of any aspect, the composition does not comprise bovine serum albumin (BSA).

In one embodiment of any aspect, each component of the composition is of pharmaceutical grade.

Another aspect described herein provides a composition for inducing a RBC comprising at least one of the components selected from the group consisting of: human AB plasma, an antibiotic, heparin, insulin, human holo-transferrin, a corticosteroid, and polyvinyl alcohol (PVA).

In one embodiment of any aspect, the composition further comprises IL-3, SCF, and EPO.

In one embodiment of any aspect, the composition further comprises SCF and EPO.

In one embodiment of any aspect, the composition further comprises EPO.

Another aspect described herein provides a method of inducing a red blood cell (RBC), the method comprising contacting a stem cell with any of the compositions described herein for a time sufficient to induce a RBC.

In one embodiment of any aspect, the RBC is an enucleated RBC.

In one embodiment of any aspect, the RBC expresses at least one cellular marker selected from GlyA (CD235a), Band 3, and CD71.

In one embodiment of any aspect, the time sufficient is at least 18 days.

In one embodiment of any aspect, the stem cell is an induced pluripotent stem cell (iPSC). In one embodiment of any aspect, the stem cell is a hematopoietic stem cell (HSC) or hematopoietic stem and progenitor cell (HSPC).

In one embodiment of any aspect, contacting occurs on an ultra-low attachment culture dish. In one embodiment of any aspect, contacting occurs at 37° C. with at least 20% 02.

In one embodiment of any aspect, the composition is replaced at least every 2 or 3 days.

Another aspect described herein provides a method of inducing a red blood cell (RBC), the method comprising (a) contacting a population of stem cells with any composition described herein; (b) contacting the population of (a) with a second composition selected from any composition described herein; and (c) contacting the population of (b) with a third composition selected from any composition described herein.

In one embodiment of any aspect, contacting is in vitro or ex vivo. In one embodiment of any aspect, contacting is culturing.

Another aspect described herein provides a RBC induced by any of the methods described herein.

Another aspect described herein provides a composition comprising any induced RBC or population thereof described herein.

In one embodiment of any aspect, the composition further comprises a pharmaceutically acceptable carrier.

Another aspect described herein provides a pharmaceutical composition comprising any induced RBC or population thereof described herein, and a pharmaceutically acceptable carrier.

In one embodiment of any aspect, the pharmaceutical composition is for use in a blood transfusion in a subject.

Another aspect described herein provides a method of treating a subject in need of a blood transfusion, the method comprising administering any induced RBC or population thereof described herein, or any composition of iRBCs described herein, or any pharmaceutical composition of iRBCs described herein to a recipient subject in need thereof.

In one embodiment of any aspect, the subject in need thereof has a disease or disorder that inhibits proper RBC formation or production.

In one embodiment of any aspect, the disease or disorder is selected from the group consisting of hemoglobinopathies (congenital abnormality of the hemoglobin molecule or of the rate of hemoglobin synthesis), examples of which include sickle-cell disease, thalassemia, and methemoglobinemia; Anemias (lack of red blood cells or hemoglobin), Pernicious anemia; disorders resulting in decreased numbers of cells, such as myelodysplastic syndrome, neutropenia (decrease in the number of neutrophils), and thrombotic thrombocytopenic purpura (TTP), thrombocytosis, hematological malignancies such as lymphomas, myelomas, and leukemia; Lymphomas such as Hodgkin's disease, Non-Hodgkin's lymphoma, Burkitt's lymphoma, Anaplastic large cell lymphoma, Splenic marginal zone lymphoma, Hepatosplenic T-cell lymphoma, and Angioimmunoblastic T-cell lymphoma (AILT); myelomas such as Multiple myeloma, Waldenstrom macroglobulinemia, Plasmacytoma; leukemias that increases defect WBC such as Acute lymphocytic leukemia (ALL), Chronic lymphocytic leukemia (CLL), Acute myelogenous leukemia (AML), Chronic Idiopathic Myelofibrosis (MF), Chronic myelogenous leukemia (CML), T-cell prolymphocytic leukemia (T-PLL), B-cell prolymphocytic leukemia (B-PLL), Chronic neutrophilic leukemia (CNL), Hairy cell leukemia (HCL), T-cell large granular lymphocyte leukemia (T-LGL), and Aggressive NK-cell leukemia.

In one embodiment of any aspect, the method further comprises, prior to administering, diagnosing a subject as having a disease or disorder that inhibits proper RBC formation or production.

In one embodiment of any aspect, the method further comprises, prior to administering, receiving the results of an assay that diagnoses a subject as having a disease or disorder that inhibits proper RBC formation or production.

In one embodiment of any aspect, the subject in need thereof has a hemoglobin level below 10 g/dL, 9 g/dL, 8 g/dL, or below 7 g/dL.

In one embodiment of any aspect, the method further comprises, prior to administering, diagnosing a subject as having hemoglobin level below 10 g/dL, 9 g/dL, 8 g/dL, or below 7 g/dL.

In one embodiment of any aspect, the method further comprises, prior to administering, receiving the results of an assay that diagnoses a subject as having hemoglobin level below 10 g/dL, 9 g/dL, 8 g/dL, or below 7 g/dL.

Another aspect described herein provides a method of treating a disease or disorder that inhibits proper RBC formation or production, the method comprising administering any induced RBC or population thereof described herein, or any composition of iRBCs described herein, or any pharmaceutical composition of iRBCs described herein to a recipient subject diagnosed as having a disease or disorder that inhibits proper RBC formation or production.

In one embodiment of any aspect, the RBC is autologous or allogenic to the subject.

Another aspect described herein provides a method of transfusing a population of autologous RBCs to a subject in need thereof, the method comprising (a) obtaining a stem cell source from a subject; (b) inducing a population of RBCs according to any of the methods described herein; and (c) administering the induced RBC population of (b) via transfusion to the subject.

Another aspect described herein provides a method of treating a disease or disorder that inhibits proper RBC formation or production, the method comprising (a) obtaining a stem cell source from a subject; (b) inducing a population of RBCs according to any of the methods described herein; and (c) administering the induced RBC population of (b) via transfusion to the subject diagnosed as having a disease or disorder that inhibits proper RBC formation or production.

In one embodiment of any aspect, the method further comprises, prior to administering, the step of genetically modifying the induced RBC of b).

In one embodiment of any aspect, the method further comprises, prior to inducing, the step of genetically modifying the stem cell source of a).

In one embodiment of any aspect, genetically modifying corrects a disease gene carried by the subject.

Another aspect described herein provides a kit comprising any of the compositions for inducing a RBC described herein and instructions for inducing a RBC using the composition.

Another aspect described herein provides a kit comprising at least two of the compositions for inducing a RBC described herein.

In one embodiment of any aspect, the kit further comprises a stem cell source.

Another aspect described herein provides a kit for inducing a RBC comprising: (a) a first composition comprising at least one of the components selected from the group consisting of: human AB plasma, an antibiotic, heparin, insulin, human holo-transferrin, a corticosteroid, and polyvinyl alcohol (PVA); (b) a second composition comprising IL-3, SCF, and EPO; (c) a third composition comprising SCF and EPO; and (d) a fourth composition comprising EPO and optionally human holo-transferrin.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

As used herein, the term “cell” refers to a single cell as well as to a population of (i.e., more than one) cells. The population may be a pure population comprising one cell type, such as a population of pluripotent stem cells or a population of induced RBCs. As used herein, the term “population” refers to a pure population or to a population comprising a majority (e.g., at least 50%, at least 60%, at least 70%, at least 80%, at least 90%, at least 95%, at least 99%) of one cell type. Alternatively, the population may comprise more than one cell type, for example a mixed cell population. It is not meant to limit the number of cells in a population; for example, a mixed population of cells may comprise at least one differentiated cell. In the present invention, there is no limit on the number of cell types that a mixed cell population may comprise.

As used herein, in one embodiment, the term “hematopoietic stem cell” or “HSC” refers to a stem cell that has self-renewal capacity and also give rise to all the blood cell types of the three hematopoietic lineages, erythroid, lymphoid, and myeloid. These cell types include the myeloid lineages (monocytes and macrophages, neutrophils, basophils, eosinophils, erythrocytes, megakaryocytes/platelets, dendritic cells), and the lymphoid lineages (T-cells, B-cells, NK-cells). Human HSCs are determined as CD34⁺, CD59⁺, CD90/Thy1⁺, CD38^{low/-}, c-kit/CD117^{-/low}, and Lin⁻. Mouse HSC- are considered CD34^{low/-}, SCA-1⁺, CD90/Thy1^{+/low}, CD38⁺, c-Kit/CD117⁺, and Lin⁻. Detecting the expression of these marker panels allows separation of specific cell populations via techniques like fluorescence-activated cell sorting (FACS). In one embodiment, the term “hematopoietic stem cell” or “HSC” refers to a stem cell that has self-renewal capacity and that have the following cell surface markers: CD34+, CD59+, Thy1/CD90⁺, CD38^{lo/-}, CD133+, c-Kit/CD117^{-/lo}, and Lin⁻. In one embodiment, the term “hematopoietic stem cell” or “HSC” refers to a stem cell that is at least CD34+. In one embodiment, the term “hematopoietic stem cell” or “HSC” refers to a stem cell that has self-renewal capacity and that is at least CD34⁺ and c-kit/CD117^{10/-}. In one embodiment, the term “hematopoietic stem cell” or “HSC” refers to a stem cell that has self-renewal capacity and that is at least CD38^{low/-}, c-kit/CD117^{-/low}. The term HSC can be used interchangeably with the term “hematopoietic stem and progenitor cell” (HSPC).

As used herein, the terms “iPS cell”, “iPSC”, and “induced pluripotent stem cell” are used interchangeably and refers to a pluripotent cell artificially derived by the transfection of the following reprogramming factors OCT4, SOX2, KLF4, and optionally c-MYC or nanog and LIN28, from a differentiated cell, e.g., a somatic cell. Alternative combinations of reprogramming factors include OCT4, SOX2, NANOG, and LIN28. The term hPSC refers to a human pluripotent stem cell.

As used herein, the term “lineage” when used in the context of stem and progenitor cell differentiation and development refers to the cell differentiation and development pathway, which the cell can take to becoming a fully differentiated cell. For example, a HSC has three hematopoietic lineages, erythroid, lymphoid, and myeloid; the HSC has the potential, i.e., the ability, to differentiate and develop into those terminally differentiated cell types known for all these three lineages. When the term “multilineage” used, it means the cell is able to, in the future, differentiate and develop into those terminally differentiated cell types known for more than one lineage. For example, the HSC has multilineage potential. When the term “limited lineage” used, it means the cell can differentiate and develop into those terminally differentiated cell types known for one lineage.

As used herein, the term “a progenitor cell” refers to an immature or undifferentiated cell that has the potential later on to mature (differentiate) into a specific cell type (a fully differentiated or terminally differentiated cell), for example, a blood cell, a skin cell, a bone cell, or hair cells. Progenitor cells have a cellular phenotype that is more primitive (e.g., is at an earlier step along a developmental pathway or progression than is a fully differentiated cell) relative to a cell, which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate. A progenitor cell also can proliferate to make more progenitor cells that are similarly immature or undifferentiated.

The term “differentiated cell” is meant any primary cell that is not, in its native form, pluripotent as that term is defined herein. The term a “differentiated cell” also encompasses cells that are partially differentiated, such as multipotent cells (e.g. adult somatic stem cells). In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell or a endodermal stem cell), which in turn can differentiate into other types of precursor cells further down the pathway (such as an tissue specific precursor, for example, a cardiomyocyte precursor, or a pancreatic precursor), and then to an end-stage differentiated cell, which plays a characteristic role in a certain tissue type, and may or may not retain the capacity to proliferate further.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult somatic stem cells, such as for example, hematopoietic stem cells and neural stem cells, hair follicle stem cells, liver stem cells etc. Multipotent means a stem cell may form many types of cells in a given lineage, but not cells of other lineages. For example, a multipotent blood stem cell can form the many different types of blood cells (red, white, platelets, etc . . . ), but it cannot form neurons; cardiovascular progenitor cell (MICP) differentiation into specific mature cardiac, pacemaker, smooth muscle, and endothelial cell types; pancreas-derived multipotent progenitor (PMP) colonies produce cell types of pancreatic lineage (cells that produces insulin, glucagon, amylase or somatostatin) and neural lineage (cells that are morphologically neuron-like, astrocytes-like or oligodendrocyte-like).

The term a “reprogramming gene”, as used herein, refers to a gene whose expression, contributes to the reprogramming of a differentiated cell, e.g. a somatic cell to an undifferentiated cell (e.g. a cell of a pluripotent state or partially pluripotent state, multipotent state). A reprogramming gene can be, for example, genes encoding master transcription factors Sox2, Oct3/4, Klf4, Nanog, Lin-28, c-myc and the like. The term “reprogramming factor” refers to the protein encoded by the reprogramming gene.

The term “isolated” as used herein signifies that the cells are placed into conditions other than their natural environment. The term “isolated” does not preclude the later use of these cells thereafter in combinations or mixtures with other cells.

As used herein, the term “expanding” refers to increasing the number of like cells through cell division (mitosis). The term “proliferating” and “expanding” are used interchangeably.

As used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to. A cell-surface marker of particular relevance to the methods described herein is CD34. An induced red blood cell (iRBC) according to the present disclosure preferably express GlyA (CD235a), Band 3, and CD71 or in other words, they are GlyA (CD235a), Band 3, and CD71 positive.

A cell can be designated “positive” or “negative” for any cell-surface marker, and both such designations are useful for the practice of the methods described herein. A cell is considered “positive” for a cell-surface marker if it expresses the marker on its cell-surface in amounts sufficient to be detected using methods known to those of skill in the art, such as contacting a cell with an antibody that binds specifically to that marker, and subsequently performing flow cytometric analysis of such a contacted cell to determine whether the antibody is bound the cell. It is to be understood that while a cell may express messenger RNA for a cell-surface marker, in order to be considered positive for the methods described herein, the cell must express it on its surface. Similarly, a cell is considered “negative” or “negative/low” (abbreviated as “−/lo” or “lo/−”) for a cell-surface marker if the cell does not express the marker on its cell surface in amounts sufficient to be detected using methods known to those of skill in the art, such as contacting a cell with an antibody that binds specifically to that marker and subsequently performing flow cytometric analysis of such a contacted cell to determine whether the antibody is bound the cell. In some embodiments, where agents specific for cell-surface lineage markers used, the agents can all comprise the same label or tag, such as fluorescent tag, and thus all cells positive for that label or tag can be excluded or removed, to leave uncontacted hematopoietic stem or progenitor cells for use in the methods described herein.

As used herein, the term “engraftment” in reference to a recipient host is when the new blood-forming cells start to grow and which are derived from the implanted cells and make healthy blood stem cells that show up in recipient's blood after a minimum period of 10 days after implantation. Engraftment can occur as early as 10 days after transplant but is more common around 14-20 days.

As used herein, the term “reconstitution” with respect to the blood system in a recipient host refers to the rebuilding the innate reservoir or working system, or part thereof within the body of recipient host to a natural or a functionally state.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cellular replacement therapy. A subject can be male or female.

A subject can be one who has been previously diagnosed with or identified as suffering from or having a condition in need of treatment (e.g. hematologic disease, etc.) or one or more complications related to such a condition, and optionally, have already undergone treatment for a hematologic disease or the one or more complications related to a hematologic disease. Alternatively, a subject can also be one who has not been previously diagnosed as having a hematologic disease or one or more complications related to a hematologic disease. For example, a subject can be one who exhibits one or more risk factors for a hematologic disease or one or more complications related to a hematologic disease or a subject who does not exhibit risk factors.

A “subject in need” of treatment for a particular condition can be a subject having that condition, diagnosed as having that condition, or at risk of developing that condition.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a hematological disease. The term “treating” includes reducing or alleviating at least one adverse effect or symptom of a condition, disease or disorder associated with a hematological disease or cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “administering,” refers to the placement of a therapeutic, e.g., cell population described herein, or composition thereof, into a subject by a method or route which results in at least partial delivery of the therapeutic at a desired site. Pharmaceutical compositions disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent or composition to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### BRIEF DESCRIPTION OF THE DRAWINGS

FIGS. 1A-1I show human plasma is crucial for the growth and development of iRBCs from human iPSCs. FIG. 1A shows a step-by-step diagram of the iPSC-iRBC differentiation protocol, starting with hemogenic differentiation of iPS cells to create CD34+ hematopoietic progenitors, followed by a three-step erythroid differentiation (ED) protocol using the EDM. Light microscope images depict the morphological characterization of cells typically seen at each ED stage. FIG. 1B is a line graph showing cell proliferation throughout the EDM protocol, measured by manual counts. *p<0.05; **p<0.01 for comparing WT plasma to serum. ^{#}<0.05; ^{##}p<0.01 for comparing SCA plasma to serum. FIG. 1C-1E are a series of line graphs showing the percentage of cells presenting with common erythroid markers, GlyA (CD235a), Band 3, and CD71 throughout the EDM protocol, measured by flow cytometry. *p<0.05; **p<0.01 for comparing WT plasma to serum. ^{#}<0.05; ^{##}p<0.01 for comparing SCA plasma to serum. FIG. 1F is a bar graph showing the percentage of enucleated iRBCs, measured by flow cytometry, counted on Days 14, 16, and 18 (mean±S.D. shown, **p<0.01). FIG. 1G is a bar graph showing the proportions of enucleated cells that were RNA-positive (reticulocytes) or RNA-negative (RBCs) by the conclusion of the EDM protocol. FIG. 1H shows images of cytospins of iRBCs, stained with May-Grunwald-Giemsa and photographed under light microscopy, showing erythroid morphological evolution at various stages of the EDM protocol. FIG. 1I shows an example of enucleation in WT iRBCs, typically seen from Day 14 onwards in a plasma-based EDM culture. Light microscopy (top) and electron microscopy (bottom) show a perforated red cell membrane where the pyrenocyte was ejected from the cell (arrowheads).

FIGS. 2A-2G show iRBCs undergo globin-switching and model human Sickle Cell Anemia in vitro. FIG. 2A is a series of line graphs showing real-time qPCR (RTqPCR) performed on WT and SCA iRBCs to measure relative globin mRNA expression throughout the EDM protocol. *p<0.05; **p<0.01. FIG. 2B shows an overlay of the expression levels of each of the beta-like globin to generate a genotype-specific map of globin-switching in iRBCs, cultured in either a normoxic or hypoxic environment. FIG. 2C shows the results of high performance liquid chromatography (HPLC) performed on WT and SCA iRBCs at Day 18 of EDM. Key eluted hemoglobin proteins are labelled. FIG. 2D is a bar graph showing the mean fractional representation of each eluted hemoglobin obtained from iRBC HPLCs. The bar areas are shown in the same top-down order as the legend; note that the level of HbS is not visible on the graph for WT iRBCs. FIG. 2E shows images of cytospins of SCA iRBCs, stained with May-Grunwald-Giemsa and photographed under light microscopy, showing iRBC morphologies under either normoxic or hypoxic incubation environments. A combination of iRBCs bearing either a “mild” or “severe” sickle phenotype was observed. Pathological findings such as sickling (see e.g., narrow arrowhead in the upper right normoxia severe panel), cell-to-cell adhesion (see e.g., black arrows in the lower right hypoxia severe panel) and haemolysis (see e.g., wide arrowhead in the lower right hypoxia severe panel) were more frequently seen in hypoxic cultures. FIG. 2F shows electron microscopic images of sickling SCA iRBCs in a hypoxic environment, showing protein filaments (arrowheads) found in the cytoplasm of the elongated edges of cells, attributed to HbS polymerization. FIG. 2G is a series of bar graphs showing the results of phalloidin permeability assays performed on peripheral blood from adult patients and iRBCs, showing the fraction of cells determined to be phalloidin-positive is significantly greater in sickled cells. **p<0.01.

FIGS. 3A-3G show RNA-sequencing identifies perturbed oxygen and metabolic pathways in SCA iRBCs. FIG. 3A Principal component analysis (PCA) of cRBCs, iRBCs, and SCA iRBCs differentiated together in comparison to publicly available (GSE53983) 24 cord blood (CB)-derived RBCs sorted at proerythroblast, basophilic, polychromatic, and orthochromatic stages of differentiation. FIG. 3B shows a heat map of Euclidean distance between all 26 samples at Day 11 and Day 15 with corresponding dendrogram depicting hierarchical clustering of samples. FIG. 3C shows a heat map of gene expression z-score for all statistically significant (p<0.05) genes across WT and SCA iRBCs. FIG. 3D is a series of bar graphs showing the p-values of enriched Gene Ontology (GO) terms from significantly up-regulated and downregulated genes in SCA iRBCs in comparison to WT iRBCs. FIG. 3E shows GSEA plots of upregulated gene sets/pathways in SCA iRBCs. FIG. 3F is a bar graph showing the fold change expression of hypoxia inducible factor-1 (HIF-1) targets genes. *p<0.05. FIG. 3G shows GSEA plots of down-regulated gene sets/pathways in SCA iRBCs.

FIGS. 4A-4H show xeno-free EDM containing PVA further enhances iRBC proliferation. FIG. 4A is a line graph showing cell proliferation of WT iRBCs grown using either a BSA- or PVA-based EDM, by manual counts. *p<0.05; **p<0.01. FIG. 4B-4C is a series of line graphs showing the percentage of WT cells, grown using either a BSA- or PVA-based EDM, presenting with common erythroid markers, GlyA (CD235a) and Band 3, throughout the EDM protocol, measured by flow cytometry. *p<0.05; **p<0.01; n.s.: not significantly different. FIG. 4D is a bar graph showing the percentage of enucleated WT iRBCs, grown using either a BSA- or PVA-based EDM, measured by flow cytometry (mean±S.D. shown, n.s.: not significantly different. FIG. 4E is a bar graph showing the proportions of enucleated cells that were RNA-positive (reticulocytes) or RNA-negative (RBCs) when grown in either a BSA- or PVA-based EDM. FIG. 4F shows the results of high performance liquid chromatography (HPLC) performed on WT iRBCs, grown in either a BSA- or PVA-based EDM. Key eluted hemoglobin proteins are labelled. FIG. 4G is a bar graph showing mean fractional representation of each eluted hemoglobin obtained from iRBC HPLCs, comparing a BSA- or PVA-based EDM. The bar areas are shown in the same top-down order as the legend. FIG. 4H shows images of May-Grunwald-Giemsa stained cytospins of Day 14 WT iRBCs grown using either a BSA- or PVA-based EDM, showing round, enucleated red blood cells with unremarkable morphology.

FIGS. 5A-5D are a series of schematics, graphs, and flow cytometry plots. FIG. 5A shows Sanger sequencing of iPS patient samples, targeting the human β-globin locus. Sequencing shows the normal genetic sequence present in the healthy (WT) patient #1157, the HBBGlu6Val missense mutation present in homozygosity in the sickle cell (SCA) patient #1347, and the correction of the HBBGlu6Val mutation back to the normal HBBGlu6 sequence in the CRISPR-corrected patient sample.

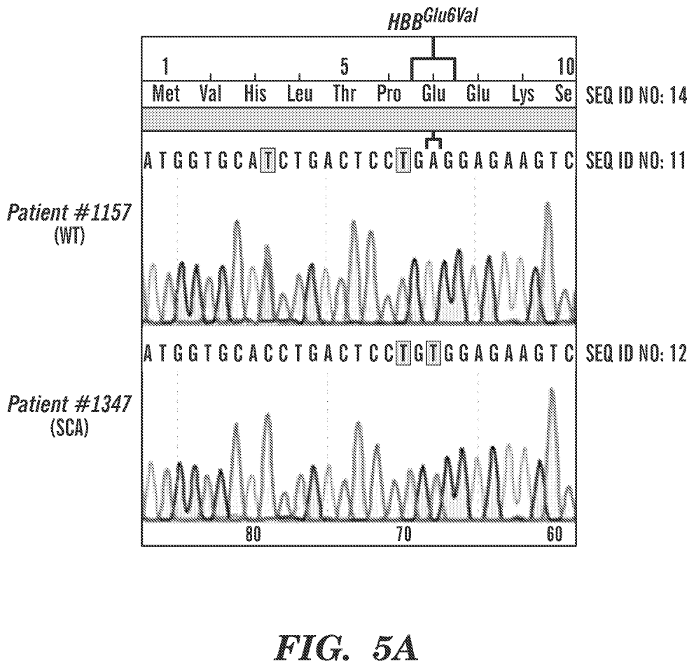

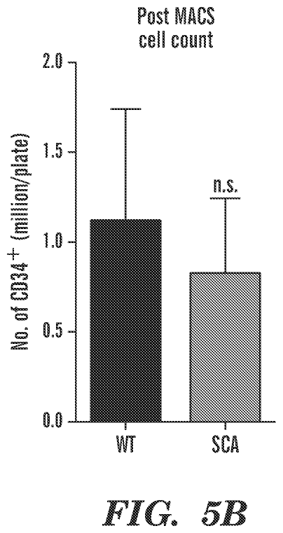

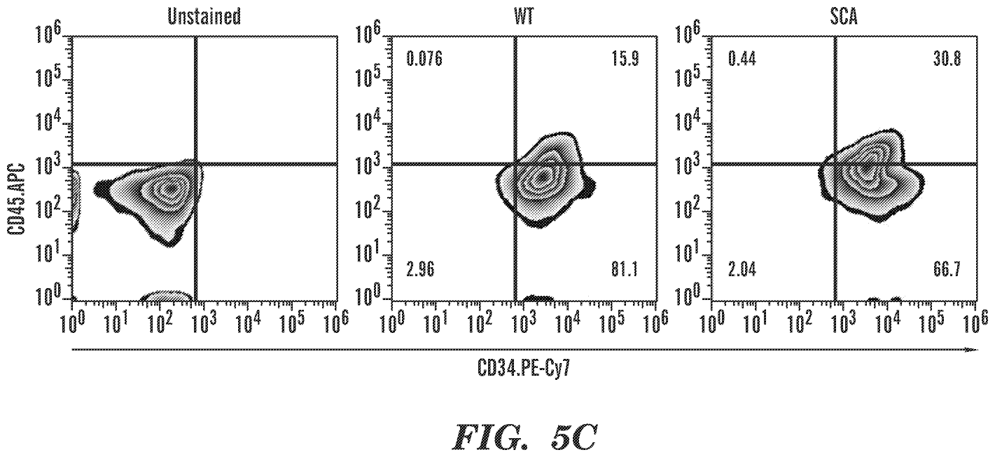

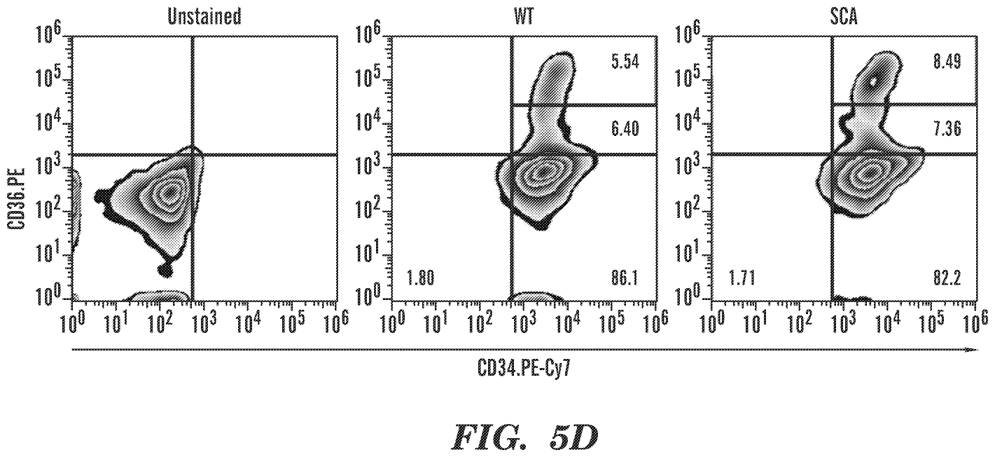

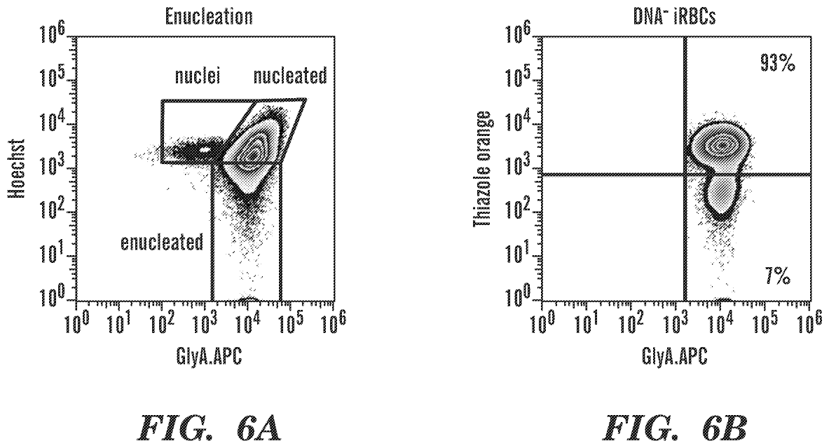

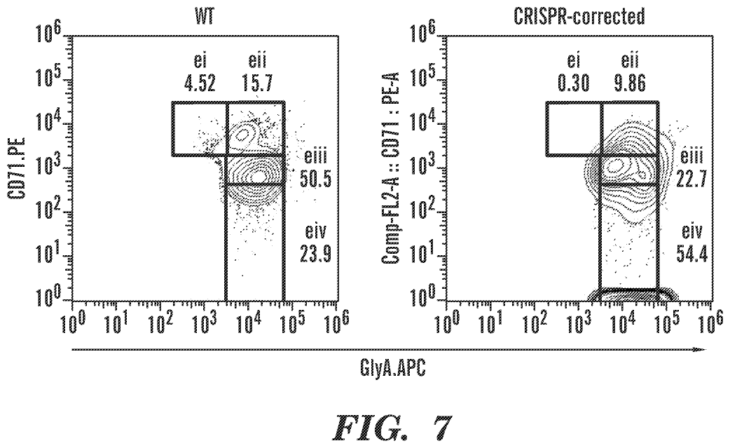

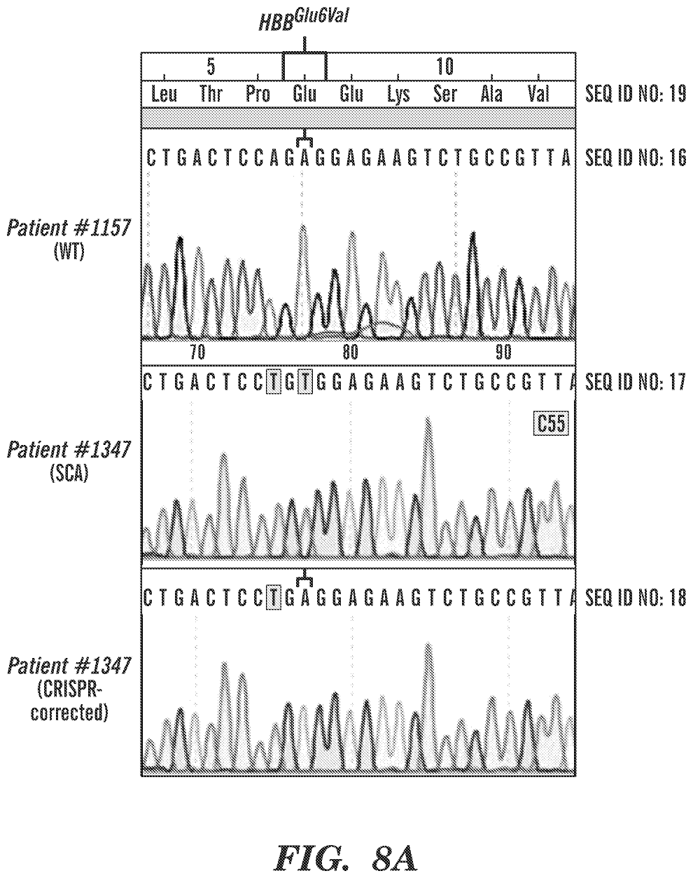

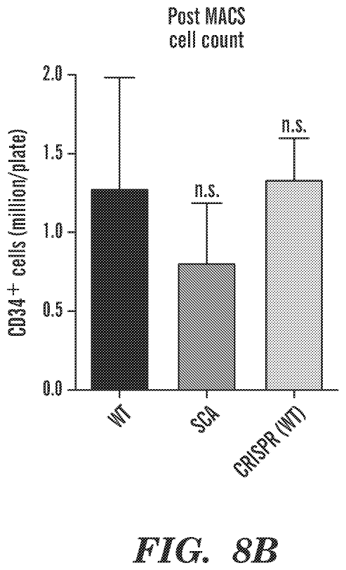

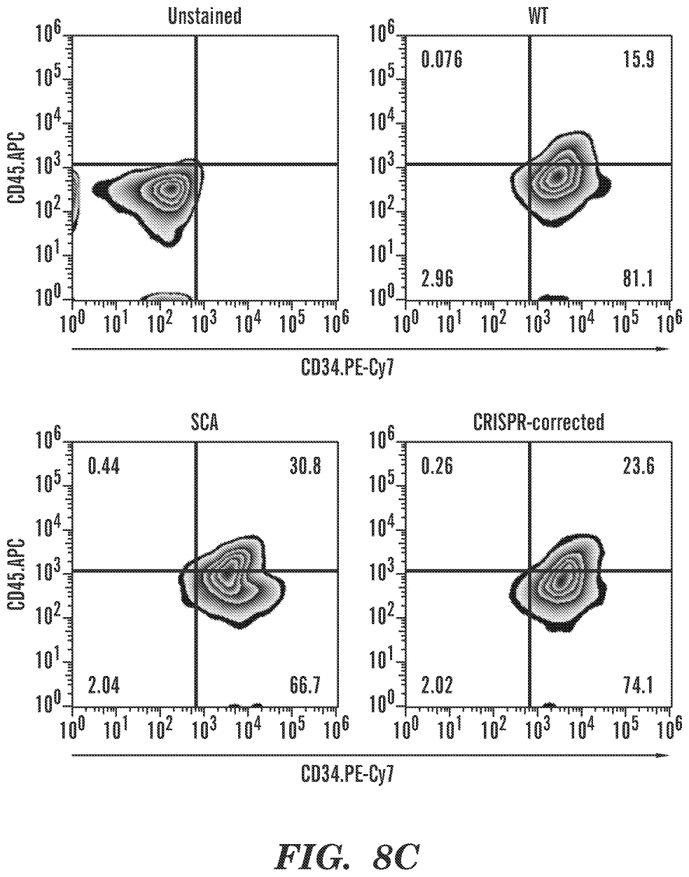

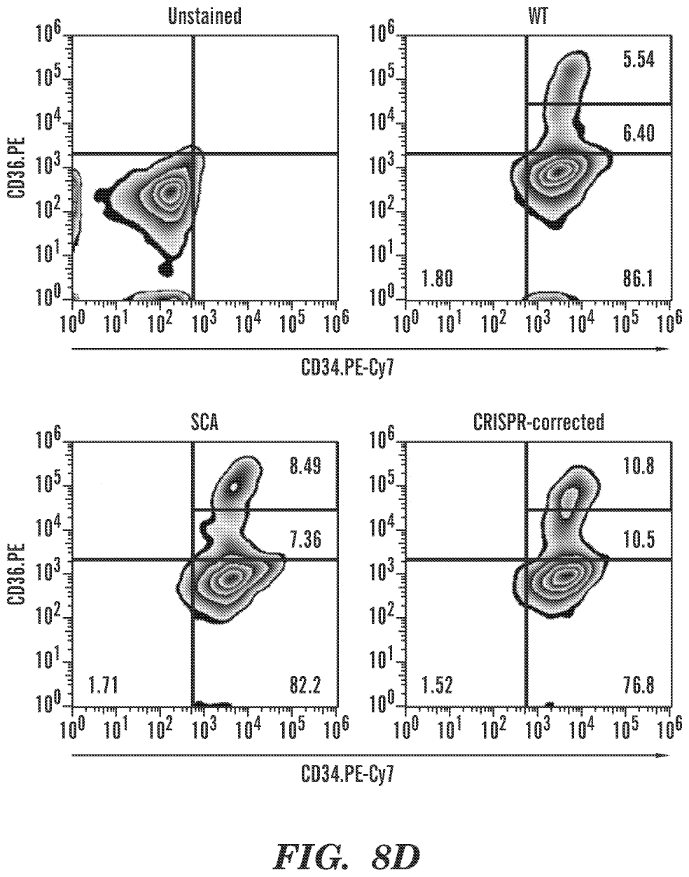

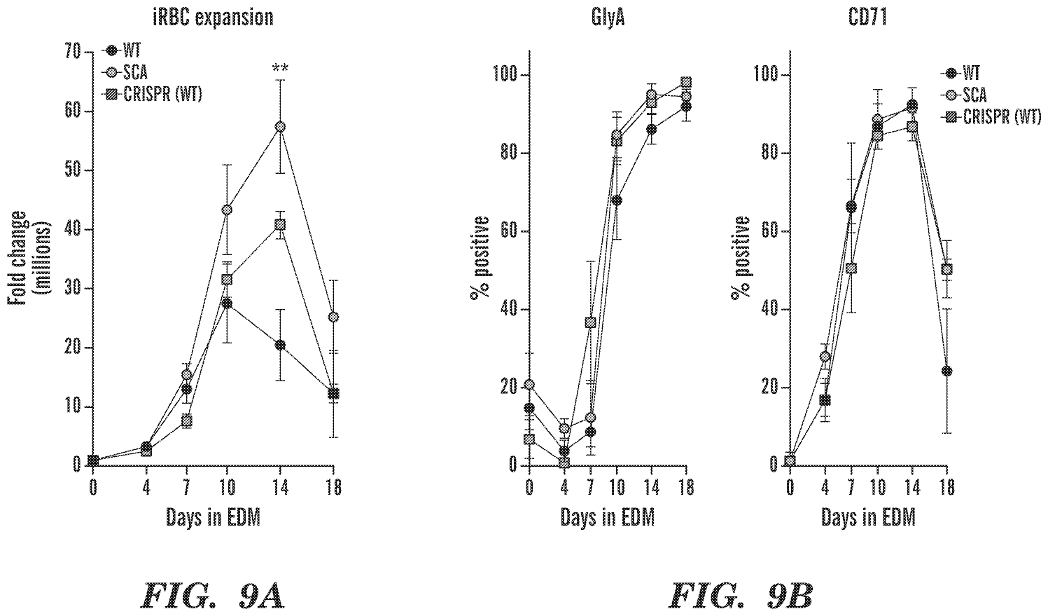

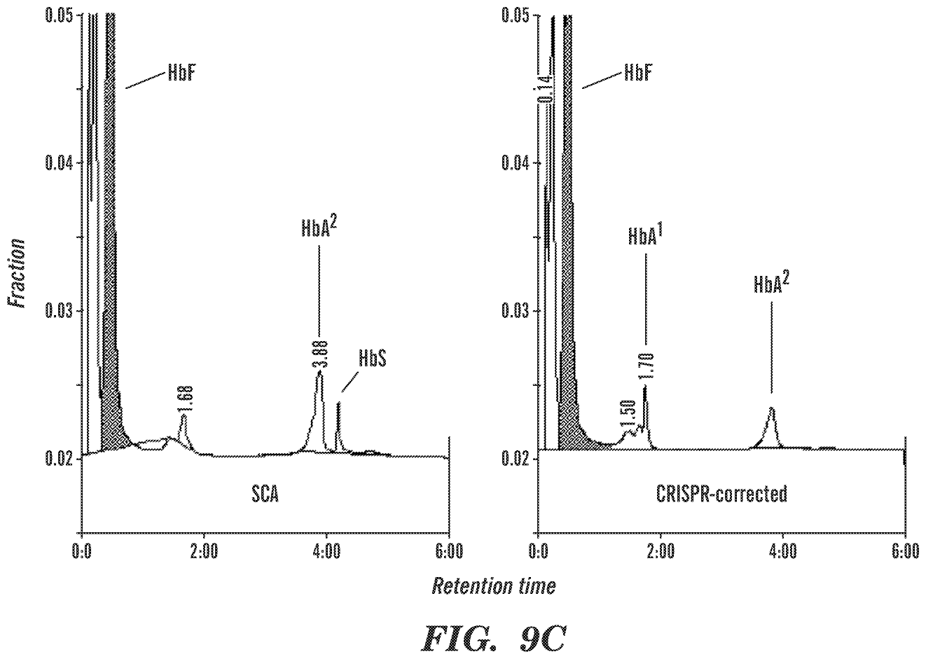

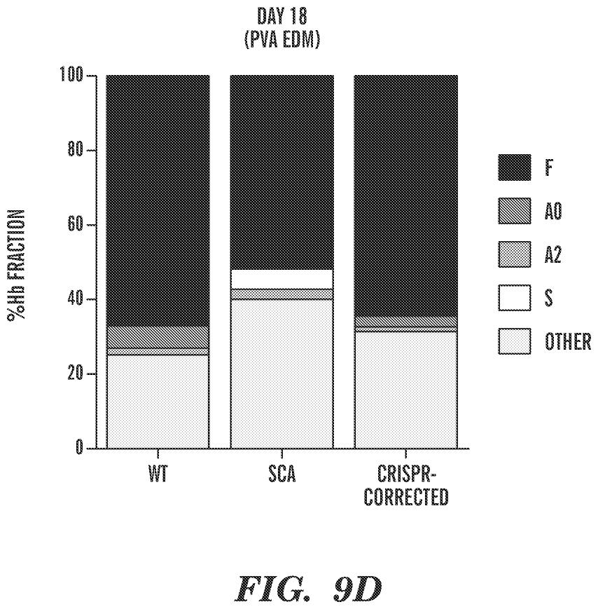

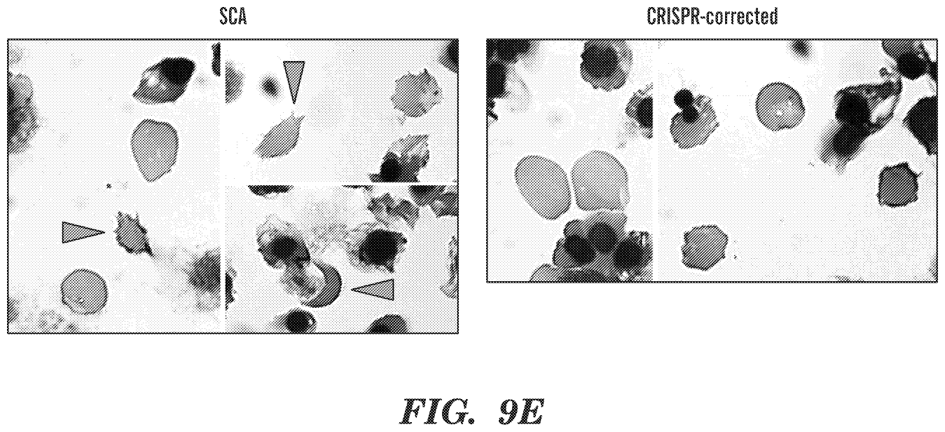

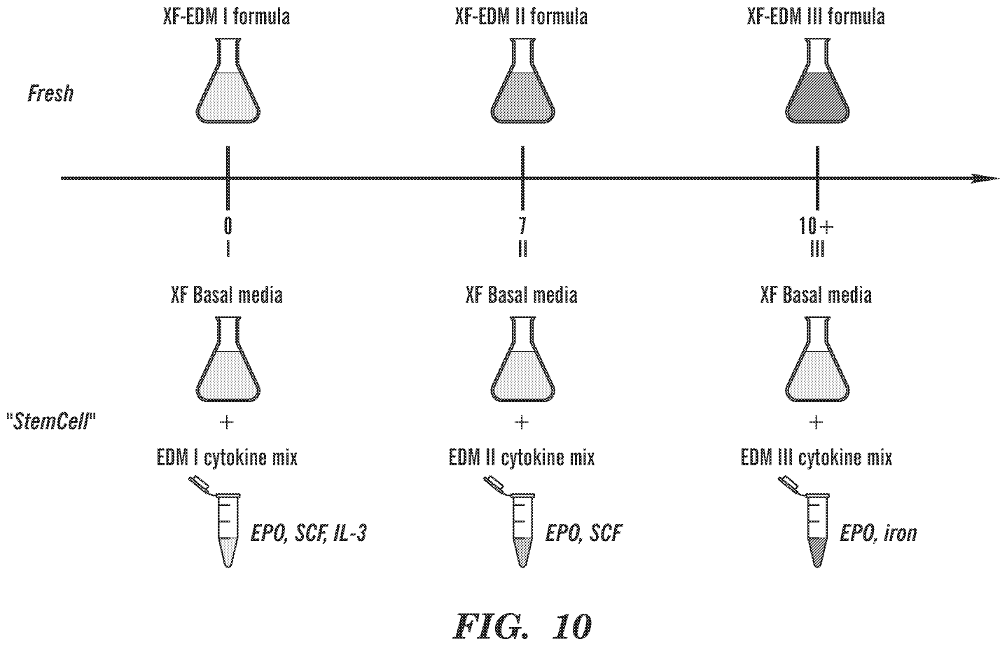

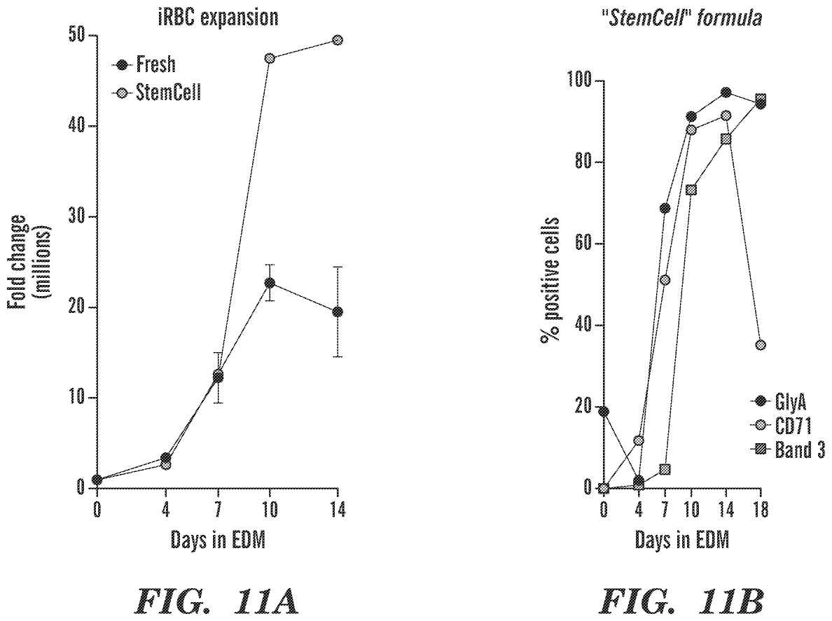

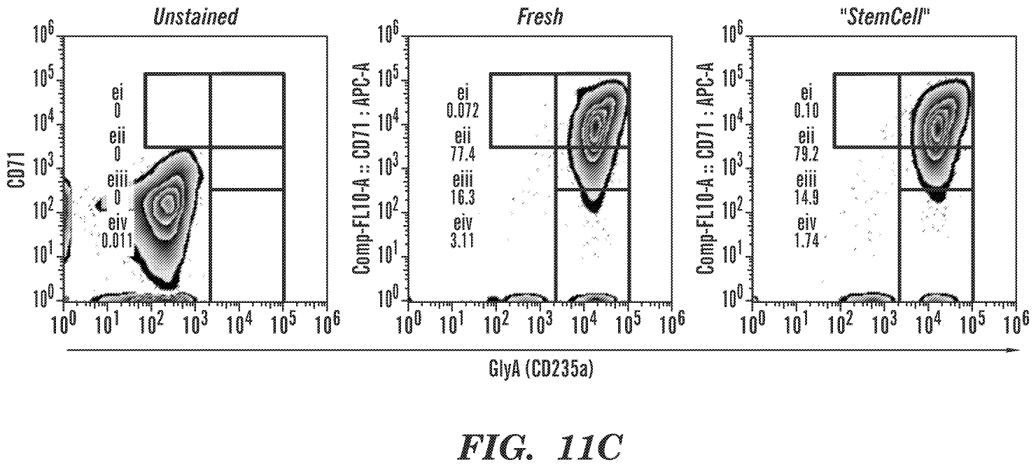

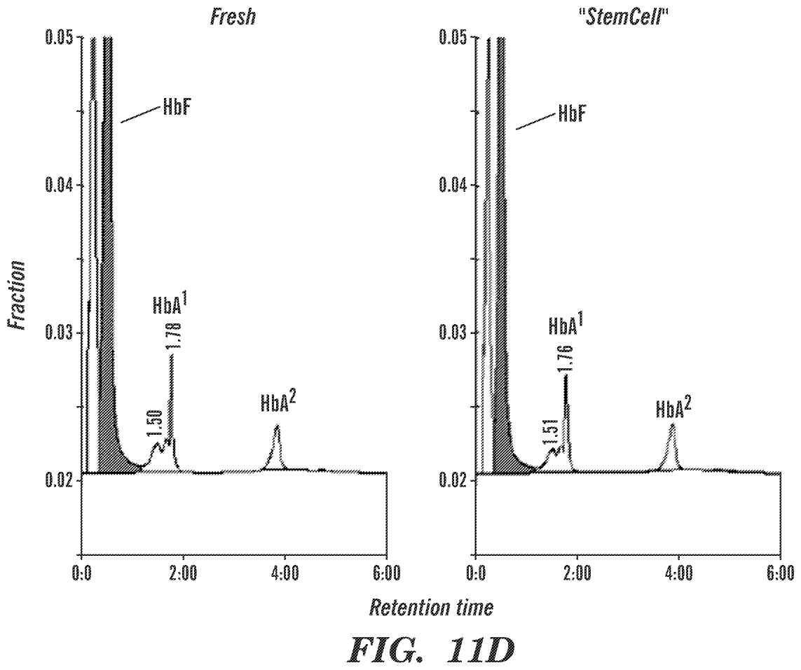

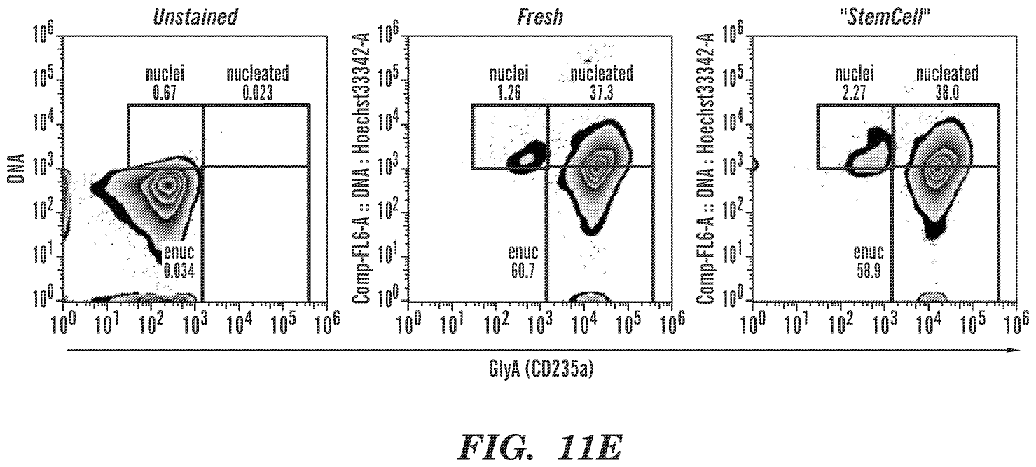

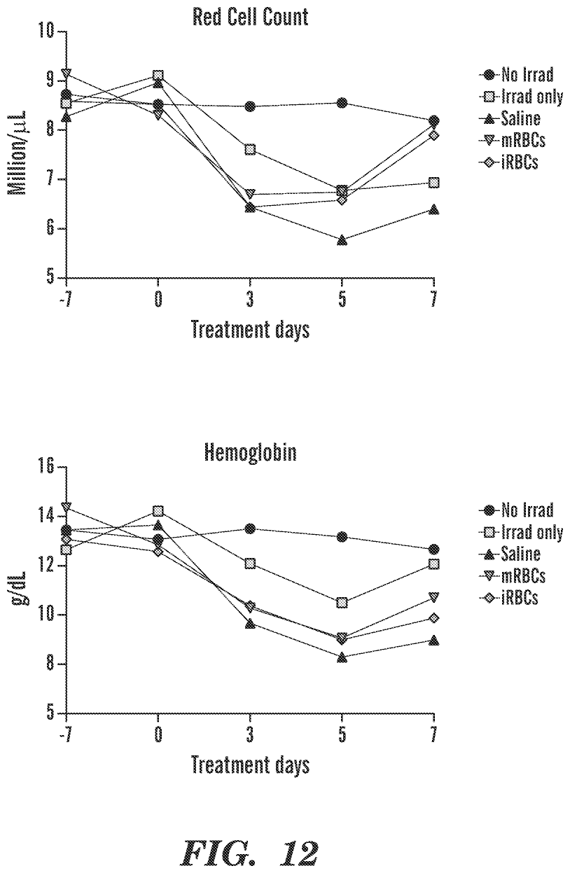

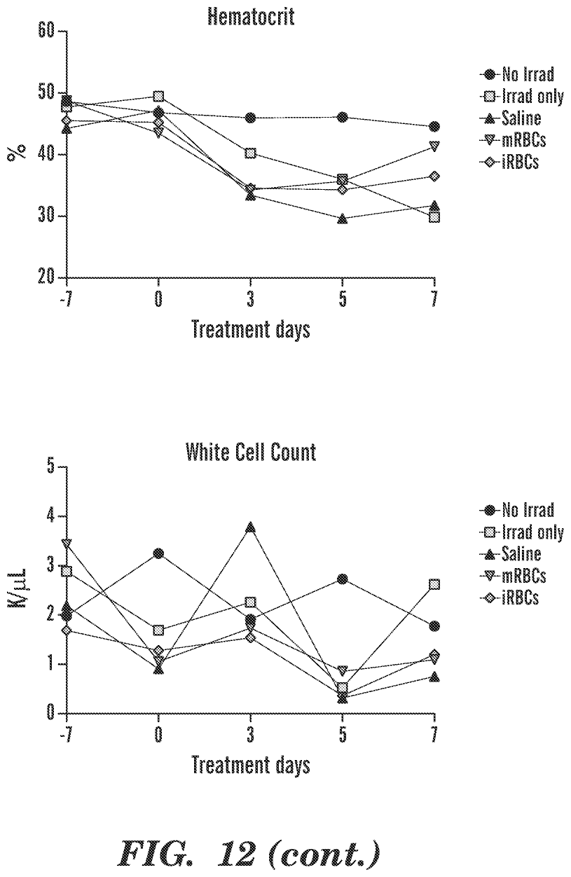

In FIG. 5A, SEQ ID NO: 11, ATGGTGCATCTGACTCCTGAGGAGAAGTC, shows the sequence of healthy (WT) patient #1157; bold double underlined text indicates the WT HBBGlu6 codon. SEQ ID NO: 14, MVHLTPEEKS, is the translation of SEQ ID NO: 11; bold double underlined text indicates WT HBBGlu6.

SEQ ID NO: 12, ATGGTGCACCTGACTCCTGTGGAGAAGTC, shows the sequence of SCA patient #1347; bold double underlined text indicates the SCA HBBGlu6Val mutant codon; italicized text shows a silent mutation in the HBBHis2 codon. SEQ ID NO: 15, MVHLTPEEKS, the translation of SEQ ID NO: 12; bold double underlined text indicates the SCA HBBGlu6Val mutant.

SEQ ID NO: 13, ATGGTGCACCTGACTCCTGAGGAGAAGTC, shows the sequence of the CRISPR-corrected patient sample from SCA patient #1347; bold double underlined text indicates the HBBGlu6Val mutation changed back to HBBGlu6; italicized text shows the silent mutation in the 2-His codon. The translation of SEQ ID NO: 13 is SEQ ID NO: 14.

FIG. 5B shows manual cell counts of the progenitor cell population (after MACS magnetic bead isolation) harvested from patient iPSCs after undergoing standard hemogenic differentiation. All iPS samples (WT, SCA, and CRISPR-corrected) produce similar numbers of hematopoietic progenitor cells per working plate. FIG. 5C shows flow cytometry performed on hematopoietic progenitor cells harvested from patient iPSCs, demonstrating all three patient cell clones produce progenitors with similar CD34/CD45 hematopoietic profiles. FIG. 5D shows flow cytometry performed on the same iPS-derived hematopoietic progenitor cells as FIG. 5C, demonstrating similar CD34/CD36 megakaryocyte-erythroid progenitor profiles. The quality and quantity of hematopoietic cells obtainable from all patient iPSC cohorts are the same.

FIGS. 6A-6B are a series of flow cytometry plots. FIG. 6A shows enucleation flow cytometry gating strategy, which demonstrates how the use of GlyA and a membrane-penetrant DNA dye (Hoechst) can be used to isolate nucleated and enucleated iRBCs, as well as highlight the projected nuclei. FIG. 6B shows thiazole orange fly cytometry gating strategy, showing how to isolate RNA-positive iRBCs (reticulocytes) from RNA-negative erythrocytes.

FIG. 7 shows flow cytometry performed on Day 14 iRBCs, comparing the GlyA/CD71 erythroid profiles of a WT patient and the CRISPR-corrected patient, showing similar erythroid maturation progression using the EDM protocol.

FIGS. 8A-8D show CRISPR-corrected cells. FIG. 8A is an image showing the results of Sanger sequencing, showing the presence of the Sickle homozygous mutation in patient #1347. After CRISPR-Cas9 intervention, the mutation has been corrected back to normal.

In FIG. 8A, SEQ ID NO: 16, CTGACTCCAGAGGAGAAGTCTGCCGTTA, shows the WT sequence of patient #1157; bold double underlined text indicates the WT HBBGlu6 codon; SEQ ID NO: 19, LTPEEKSAV, is the translation of SEQ ID NO: 16; bold double underlined text indicates WT HBBGlu6.

SEQ ID NO: 17, CTGACTCCTGTGGAGAAGTCTGCCGTTA, shows the SCA sequence of patient #1347; bold double underlined text indicates the SCA HBBGlu6Val mutant codon; SEQ ID NO: 20, LTPYEKSAV, is the translation of SEQ ID NO: 17; bold double underlined text indicates SCA HBBGlu6Val mutant codon.

SEQ ID NO: 18, CTGACTCCTGAGGAGAAGTCTGCCGTTA, shows the CRISPR-corrected sequence of patient #1347; bold double underlined text indicates the HBBGlu6Val mutation changed back to HBBGlu6; the translation of SEQ ID NO: 18 is SEQ ID NO: 19.

FIG. 8B is a bar graph showing a post-MACS sort TRYPAN blue count after using STEMCELL's STEMDIFF to collect CD34+ cells from the three iPS samples. There was no difference shown. FIG. 8C shows a typical post-MACS CD34/CD45 flow cytometry panel. The higher CD45 reading on the SCA is likely related to the age of the original patient (young) versus the typical 1157 patient (much older). Since the CRISPR-corrected sample is also high in CD45, this is considered an age-related finding, not genotype-related. FIG. 8D shows a similar FACS panel to FIG. 8C but with CD36 hi/low also analyzed, to look for Erythroid/Megakaryoid progenitors.

FIGS. 9A-9E show expansion of CRISPR-corrected cells. FIG. 9A is a line graph showing cell growth in all three patient cohorts. The CRISPR-corrected sample shows cell growth due to age. FIG. 9B are a series of line graphs showing the results of GlyA and CD71 flow cytometry; there was no significant difference found. FIG. 9C shows the results of HPLC, comparing patient #1347 (Sickle cell) and their CRISPR-corrected cells. HbS is markedly reduced, and HbA is increased in CRISPR-corrected sample as compared to SCA, confirming that the correction has occurred and the disease has been eliminated. FIG. 9D is a bar graph showing collected data from all HPLCs. F=HbF; A0=HbA1; A2=HbA2; S=HbS. Sickle cell patients do not make HbAl. The bar areas are shown in the same top-down order as the legend. FIG. 9E shows images of cytospins, stained with MGG. Sickle cell are mild in phenotype but present in the SCA sample (see arrows). No sickle cells were seen in CRISPR-corrected cells.

FIG. 10 shows schematic of “StemCell” formula versus fresh formula.

FIG. 11A-11E show RBC induction via “StemCell” formula versus fresh formula. FIG. 11A show manual cell counts showing RBC induction. FIG. 11B show flow cytometry for key erythroid markers of cells induced via indicated formula. FIG. 11C is a series of flow cytometry plots showing the developmental profile at day 14 for erythroid cells induced via indicated formula. FIG. 11D shows HPLC results at day 16 of cells induced via indicated formula. FIG. 11E is a series of plots showing the enucleation of cells induced via indicated formula, measured by flow cytometry.

FIG. 12 is a series of line graphs showing red blood cell statistics and key indices from the iRBC transfusion trial (e.g., red cell count, hematocrit, hemoglobin, white blood cell count). Mice that received 300cGy of radiation at Day 0 saw a decline in many red cell parameters, resulting in anemia. Mice then treated on Day 3 with a single transfusion of either mouse red cells (mRBCs) or human iRBCs experienced a positive recovery in the days following. Mice given no treatment (Irrad only), or just Saline, experienced a delayed/poor recovery from radiation-induced anemia. N=1 for each cohort. There was no correlation between white blood cell counts and any treatment groups. The RBC transfusions did not affect the indices of other blood types in the mice; only red cell related values were affected by transfusion. Blood stats were obtained by HEMAVET (DREW SCIENTIFIC).

### DETAILED DESCRIPTION

Human induced pluripotent stem cells (iPSC) are an invaluable resource in tissue and blood cell engineering due to their multi-lineage potential in culture systems. iPSCs that undergo hemogenic differentiation in vitro generate hematopoietic progenitor cells capable of differentiation into a variety of mature blood cell lineages if supplied the correct growth factors, cytokines, and hormones. The generation of adult red blood cells (erythrocytes) from these sources has historically been difficult, often due to a lack of complete maturation, enucleation, or the expression of adult globin chains. The compositions described herein, also known as Xeno-Free Erythroid Differentiation Media (XF-EDM), has been shown to produce large numbers of mature, enucleated RBCs from non-blood cell source material (iPSCs). These artificially-grown cells are called induced RBCs (iRBCs). iRBCs of the invention are the first adult RBC products generated in a laboratory that are safe for live human use. This important step towards engineering a clinically usable blood-replacement product is largely due to the incorporation of a non-toxic chemical compound called polyvinyl alcohol (PVA), which serves as a replacement for an animal-derived product called bovine serum albumin (BSA) found in former published formulas for red cell engineering in culture. The XF-EDM of the invention containing PVA is an enhanced formula for generating human blood cells with greater potential to be accepted as a therapeutic avenue for patients who rely on live blood donations.

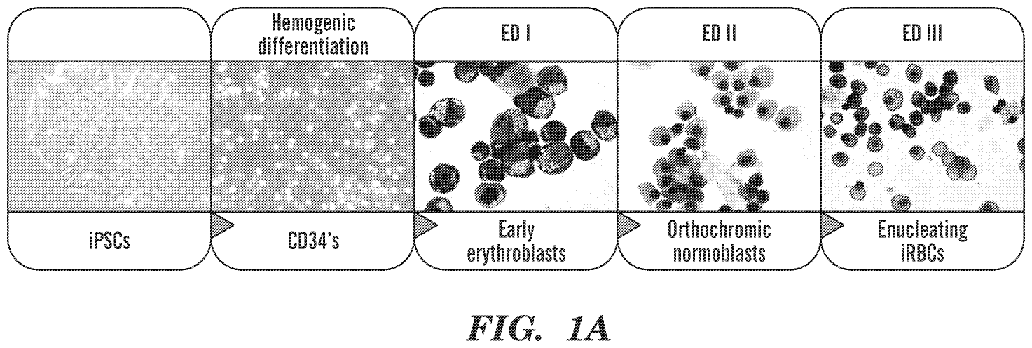

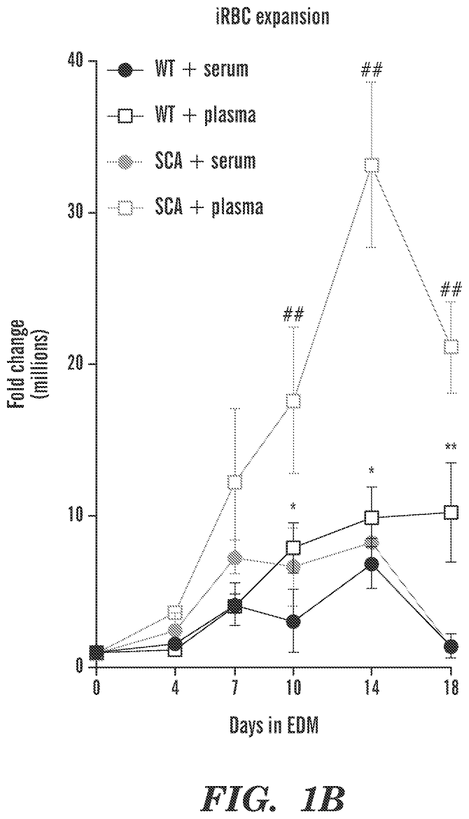

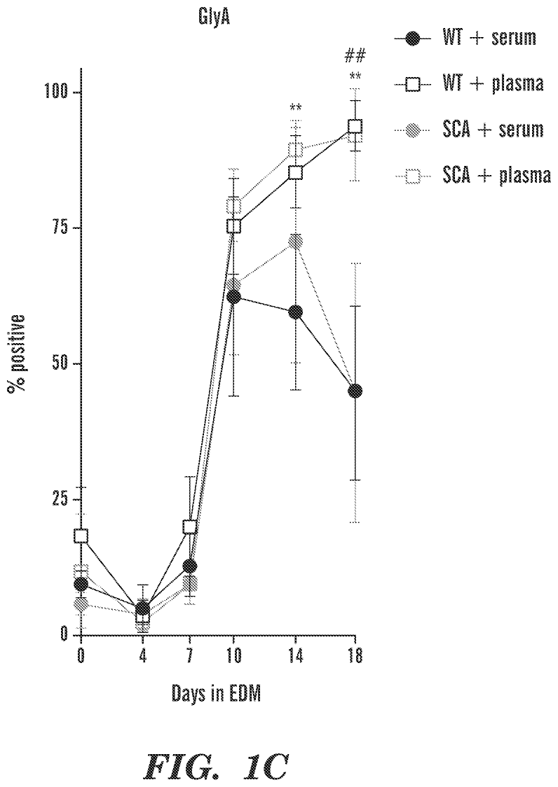

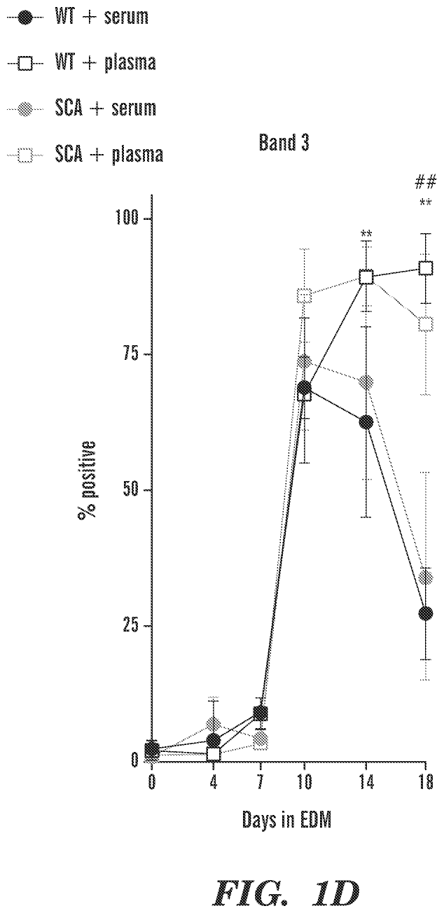

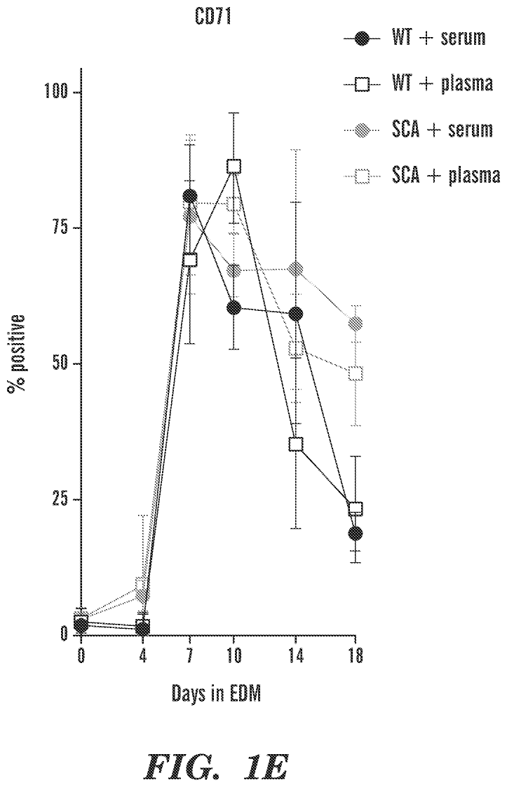

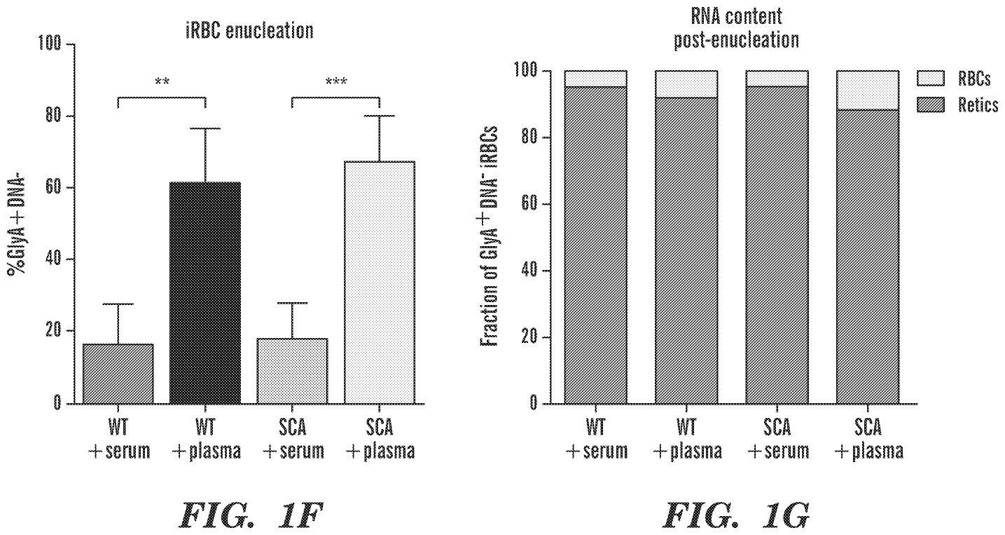

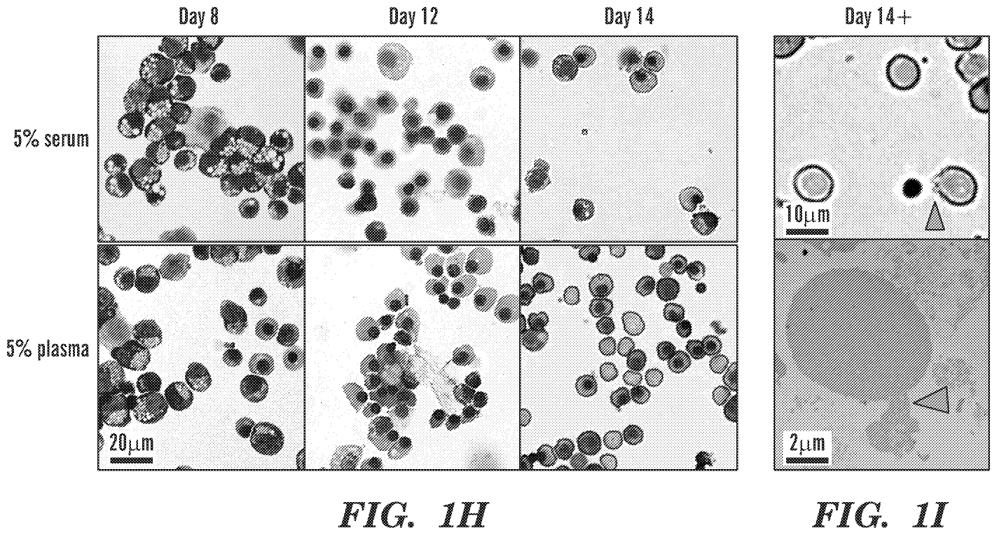

The compositions, e.g., XF-EDM, and methods described herein are designed to be applied to human hematopoietic progenitor cells (often called CD34⁺ cells), which are obtainable from, e.g., fetal cord blood and adult bone marrow, or derived from patient iPSCs after hemogenic differentiation. The protocol takes place over three distinct erythroid development (ED) stage, e.g., ED I, ED II, and ED III. Throughout these stages, the hematopoietic progenitor cells expand to large numbers and differentiate down the erythroid lineage, gradually developing into mature, enucleated iRBCs (see e.g., FIG. 1).

### Compositions for Inducing Blood Cells

Aspects described herein provide compositions for inducing a blood cell. In one embodiment, the blood cell is a RBC. In another embodiment, the blood cell is a cell of erythroid lineage. A “cell of the erythroid lineage” refers to a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage,” as the term is used herein, comprise hematopoietic progenitor cells, rubriblasts, prorubricytes, erythroblasts, metarubricytes, reticulocytes, and erythrocytes.

One aspect of the invention is a composition for the induction of a RBC, comprising human AB plasma; heparin; insulin; holo-transferrin; a corticosteroid; and polyvinyl alcohol (PVA).

One aspect of the invention is a composition for the induction of a RBC, comprising at least one component selected from the group consisting of human AB plasma; antibiotic; heparin; insulin; holo-transferrin; a corticosteroid; and polyvinyl alcohol (PVA). For example, the composition can comprise at least 2, 3, 4, 5, 6, or 7 components.

In various embodiments, the composition further comprises at least one growth factor, e.g., required for the induction of a RBC. For example, IL-3, stem cell factor (SCF) and erythropoietin (EPO).

In one embodiment, the composition comprises human AB plasma; heparin; insulin; human holo-transferrin; a corticosteroid; PVA, IL-3, SCF, and EPO.

In one embodiment, the composition comprises human AB plasma; heparin; insulin; human holo-transferrin; a corticosteroid; PVA, SCF, and EPO.

In one embodiment, the composition comprises human AB plasma; heparin; insulin; human holo-transferrin; a corticosteroid; PVA, and EPO.

The methods described herein require three distinct compositions for each erythroid development (ED) stage, e.g., ED I, ED II, and ED III. One aspect provides a composition for ED I comprising human AB plasma; heparin; insulin; human holo-transferrin; a corticosteroid; PVA, IL-3, SCF, and EPO.

One aspect provides a composition for ED II comprising human AB plasma; heparin; insulin; human holo-transferrin; a corticosteroid; PVA, SCF, and EPO.

One aspect provides a composition for ED III comprising human AB plasma; heparin; insulin; human holo-transferrin; a corticosteroid; PVA, and EPO.

In one embodiment, any of the compositions further comprises at least one antibiotic, e.g., Penicillin and/or Streptomycin. Antibiotics are used in culture media to reduce or prevent bacterial contamination in the culture system. A skilled person would be able to select an appropriate antibiotic for a given composition. In certain embodiment, the at least one antibiotic is present in the composition at a concentration of at least 1%, 2%, 3%, 4%, 5%, 6%, 7% or more.

In one embodiment, the composition further comprises Iscove's DMEM (IMDM). IMDM is commercially available from, e.g., THERMOFISHER, Waltham, MA.

Polyvinyl alcohol (also known as PVOH, PVA, or PVA1) is a water-soluble synthetic polymer and has the idealized formula of [CH2CH(OH)]. PVA in an inert component of culture media that serves as a replacement for bovine serum albumin (BSA). Much like albumin, PVA performs a number of “buffer” functions that maintains the media's integrity. It has been shown to balance pH, serves as an anti-oxidant, an anti-coagulant, and stabilizes important hormones and growth factors in the media that otherwise degrade very rapidly. In one embodiment, the concentration of PVA in the composition is 0.08%. In one embodiment, the concentration of PVA in the composition is less than 0.08%, e.g., 0.01%, 0.02%, 0.03%, 0.04%, 0.05%, 0.06%, or 0.07%. In one embodiment, the concentration of PVA in the composition is greater than 0.08%, e.g., 0.09%, 0.1%, 0.12%, 0.13%, 0.14%, 0.15%, or 0.16%.

Human AB plasma is a vital source of nutrients, sugars, amino acids, and other organic molecules that serve as pro-viability factors (e.g., keeps the stem cell culture alive; prevents cellular death), and pro-erythropoietic factors (e.g., contains erythroid-specific growth factors that speed up the transformation of stem cells into RBCs). Data provided herein demonstrate that a stem cell source grown in a composition lacking human AB plasma prematurely die and fail to fully mature into RBCs. In one embodiment, the concentration of heat shocked human plasma or human AB serum in the composition is 0.5%, 1%, 2%, 3%, 4%, 5%, 6%, 7%, 8%, 9%, 10%, or more.

Heparin is an anti-coagulant required to keep the blood cell culture in suspension. In one embodiment, the concentration of human heparin in the composition is 0.5U, 1 U, 2U, 2.1U, 2.25U, 2.5U, 2.75U, 3U, 3.25U, 3.5U, 3.75U, 4U, 5U, 6U, 7U, 8U, 9U, 10U, or more.

Insulin is a growth factor that helps regulate cellular metabolism (e.g., sugar breakdown) and promotes growth and differentiation of several cell types, including erythroid cells. In one embodiment, insulin is present in the composition at a concentration of at least 1 ug/mL, at least 2 ug/mL, at least 3 ug/mL, at least 4 ug/mL, at least 5 ug/mL, at least 6 ug/mL, at least 7 ug/mL, at least 8 ug/mL, at least 9 ug/mL, at least 10 ug/mL, at least 11 ug/mL, at least 12 ug/mL, at least 13 ug/mL, at least 14 ug/mL, at least 15 ug/mL, at least 16 ug/mL, at least 17 ug/mL, at least 18 ug/mL, at least 19 ug/mL, at least 20 ug/mL, or more.

Holo-transferrin is an iron-containing protein that is absolutely necessary for hemoglobin synthesis within erythroid cells. Erythroid cells absorb Holo-transferrin through the Transferrin Receptor, and then integrate it into the hemoglobin protein. Hemoglobin synthesis does not occur without adequate levels of Holo-transferrin. In one embodiment, human holo-transferrin is present in the composition at a concentration of at least 50 ug, 100 ug, 150 ug, 200 ug, 250 ug, 300 ug, 350 ug, 400 ug, 450 ug, 500 ug, 550 ug, 600 ug, 650 ug, 700 ug, 750 ug, 800 ug, 850 ug, 900 ug, 950 ug, 1000 ug, or more per mL.

Corticosteroids have a known positive effect on RBC production from their stem cells source, and are used to treat a number of bone marrow failure syndromes to boost blood numbers in circulation. An exemplary corticosteroid, dexamethasone, increases the number of RBCs acquired per progenitor cell. It maximizes their erythroid proliferative power, increasing the total number of RBCs obtainable through this method. In one embodiment, the corticosteroid is dexamethasone. In one embodiment, any corticosteroid known in the art can be used in the compositions described herein. In certain embodiments, dexamethasone is present at a concentration of at least 100 nM, 200 nM, 300 nM, 400 nM, 500 nM, 600 nM, 700 nM, 800 nM, 900 nM, or more.

The iRBCs of the invention are safe for live human use. Accordingly, in one embodiment, the composition does not contain BSA. In one embodiment, BSA is not use in any of the methods or compositions described herein.

In one embodiment, the composition comprises IMDM (Iscove's DMEM, CORNING, USA), 2% Pen/Strep (CORNING, USA), 5% human AB serum (VALLEY BIOMEDICAL INC, USA), 3 U human heparin (ACROS DIAGNOSTICS, Belgium), 10 μg/mL insulin (SIGMA-ALDRICH, USA), 250 μg human holo-transferrin (SIGMA-ALDRICH, USA), and 500 nM dexamethasone (SIGMA-ALDRICH, USA).

In various embodiments, the composition further comprises at least one growth factor, e.g., required for the induction of a RBC. For example, IL-3, stem cell factor (SCF) and erythropoietin (EPO). In some embodiments, IL3 and/or SCF are provided in the composition at a concentration of at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 11 ng/mL, at least 12 ng/mL, at least 13 ng/mL, at least 14 ng/mL, at least 15 ng/mL, at least 16 ng/mL, at least 17 ng/mL, at least 18 ng/mL, at least 19 ng/mL, at least 20 ng/mL, at least 25 ng/mL, at least 30 ng/mL, at least 35 ng/mL, at least 40 ng/mL or more. In some embodiments, EPO is provided in the composition at a concentration of at least 0.5 U/mL, at least 0.1 U/mL, 0.2 U/mL, 0.3 U/mL, 0.4 U/mL, 0.5 U/mL, 0.6 U/mL, 0.7 U/mL, 0.8 U/mL, 0.9 U/mL, 1 U/mL, 1.1 U/mL, 1.2 U/mL, 1.3 U/mL, 1.4 U/mL, 1.5 U/mL, 1.6 U/mL, 1.7 U/mL, 1.8 U/mL, 1.9 U/mL, 2 U/mL, 2.1 U/mL, 2.2 U/mL, 2.3 U/mL, 2.4 U/mL, 2.5 U/mL, 2.6 U/mL, 2.7 U/mL, 2.8 U/mL, 2.9 U/mL, 3 U/mL, 3.1 U/mL, 3.2 U/mL, 3.3 U/mL, 3.4 U/mL, 3.5 U/mL, 3.6 U/mL, 3.7 U/mL, 3.8 U/mL, 3.9 U/mL, or more. The concentrations of IL-3, SCF and EPO should be used such that they promote the differentiation of hemogenic endothelium into a population of RBCs.

The concentration of IL3, SCF and/or EPO can be the same or different. In one embodiment, the concentration of IL3, SCF and/or EPO is different in each composition, e.g., ED I, ED II, and ED III.

In one embodiment, the composition for ED I comprises IMDM (Iscove's DMEM, CORNING, USA), 2% Pen/Strep (CORNING, USA), 5% human AB serum (VALLEY BIOMEDICAL INC, USA), 3 U human heparin (ACROS DIAGNOSTICS, Belgium), 10 μg/mL insulin (SIGMA-ALDRICH, USA), 250μg human holo-transferrin (SIGMA-ALDRICH, USA), 500 nM dexamethasone (SIGMA-ALDRICH, USA), 2 ng/mL IL-3 (R&D SYSTEMS, USA), 10 ng/mL SCF (R&D SYSTEMS, USA), and 3 U/mL EPO (LIFE TECHNOLOGIES, USA).

In one embodiment, the composition for ED II comprises IMDM (Iscove's DMEM, CORNING, USA), 2% Pen/Strep (CORNING, USA), 5% human AB serum (VALLEY BIOMEDICAL INC, USA), 3 U human heparin (ACROS DIAGNOSTICS, Belgium), 10 μg/mL insulin (SIGMA-ALDRICH, USA), 250 μg human holo-transferrin (SIGMA-ALDRICH, USA), 500 nM dexamethasone (SIGMA-ALDRICH, USA), 10 ng/mL SCF, and 1 U/mL EPO.

In one embodiment, the composition for ED III comprises IMDM (Iscove's DMEM, CORNING, USA), 2% Pen/Strep (CORNING, USA), 5% human AB serum (VALLEY BIOMEDICAL INC, USA), 3U human heparin (ACROS DIAGNOSTICS, Belgium), 10 μg/mL insulin (SIGMA-ALDRICH, USA), 250 μg human holo-transferrin (SIGMA-ALDRICH, USA), 500 nM dexamethasone (SIGMA-ALDRICH, USA), 0.2 U/mL EPO, 300 m/mL additional human holo-transferrin.

In one embodiment, each component of a composition described herein is pharmaceutical grade, meaning the component exceeds 99% purity (natural sources) and contain no binders, fillers, excipients, dyes, or unknown substances.

In another embodiment, the composition for inducing RBCs is made fresh for each differentiated stage, e.g., on the day it is required in the methods. For example, the basal media is prepared the day of, and the components (i.e., the stage-specific cytokines) are added to fresh basal media the day it is required, and stored at 4° C. This method is referred to herein as “fresh” formula.

In another embodiment, all basal media used for inducing RBCs is prepared at the beginning of the methods and stored at 4° C. This premade basal media is used at all differentiated stages. 3 vials of EDM cytokine mixes, which correspond to each differentiation stage, are prepared and stored at −20° C. The frozen cytokine mixtures are thawed and added to premade basal media at the day it is required. This method is referred to herein as “StemCell” formula.

Crystalized PVA is added to the liquid basal formula and requires high humidity to dissolve. A 37° C. water bath is typically used for approximately 30mins to ensure PVA mixes into the formula. The “fresh” formula contains all media components (including temperature-sensitive cytokines, hormones, and growth factors), and in a heat bath, some of these components may partially degrade in the first 30min, reducing their potency. Using the “StemCell” formula, the PVA is dissolved into the media using heat first, and then the cytokines/growth factors are added later, immediately prior to using the media on the cells. The cytokines/growth factors avoid this long exposure to heat. This approach results in a higher growth rates of red blood cells generated using the “StemCell” formula as compared to the fresh formula.

### Methods for Inducing a Red Blood Cell (RBC)

In one aspect, described herein is a method comprising contacting a stem cell, e.g., an iPSC or a HSC, with any of the compositions described herein for a time sufficient to induce a RBC.

In one embodiment, the time sufficient is at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, or more.

In one embodiment, the stem cell source is contacted with the same composition throughout the time sufficient. In an alternative embodiment, the cells are contacted with different compositions throughout the time sufficient.

For example, in one embodiment, the stem cell source is contacted with the composition for ED I for at least 1 day, at least 2 days, at least 3 days, at least 4 days, at least 5 days, at least 6 days, at least 7 days, at least 8 days, at least 9 days, at least 10 days promote induction into a RBC.

In one embodiment, the stem cell source is contacted with the composition for ED II for at least 1 day, at least 2 days, at least 3 days, at least 4 days, at least 5 days, at least 6 days, at least 7 days, at least 8 days, at least 9 days, at least 10 days promote induction into a RBC.

In one embodiment, the stem cell source is contacted with the composition for ED III for at least 1 day, at least 2 days, at least 3 days, at least 4 days, at least 5 days, at least 6 days, at least 7 days, at least 8 days, at least 9 days, at least 10 days promote induction into a RBC.

In one embodiment, the stem cell source is contacted with the composition for ED I for at least 7 days, with the composition for ED II for at least 3 days, and with the composition for ED III for at least 8 days to promote induction into a RBC. In one embodiment, the stem cell source is contacted sequentially: (1) with the composition for ED I for at least 7 days, (2) with the composition for ED II for at least 3 days, and (3) with the composition for ED III for at least 8 days to promote induction into a RBC.

In certain embodiment, fresh media is added to the cells every 2-3 days throughout contacting.

In one embodiment, contacting occurs on an ultra-low attachment culture dish. In one embodiment, contacting occurs at 37° C. with at least 20% O₂.

In one embodiment, contacting can be in vitro or ex vivo.

In one embodiment, 0.5×10⁵/mL to 2.5×10⁵/mL stem cells are contacted with the composition for ED I. As a non-limiting example, at least 0.5×10⁵/mL, at least 0.6×10⁵/mL, at least 0.7×10⁵/mL, at least 0.8×10⁵/mL, at least 0.9×10⁵/mL, at least 1.0×10⁵/mL, at least 1.1×10⁵/mL, at least 1.2×10⁵/mL, at least 1.3×10⁵/mL, at least 1.4×10⁵/mL, at least 1.5×10⁵/mL, at least 1.6×10⁵/mL, at least 1.7×10⁵/mL, at least 1.8×10⁵/mL, at least 1.9×10⁵/mL, at least 2.0×10⁵/mL, at least 2.1×10⁵/mL, at least 2.2×10⁵/mL, at least 2.3×10⁵/mL, at least 2.4×10⁵/mL, or at least 2.5×10⁵/mL, or more stem cells are contacted with the composition for ED I.

In one embodiment, 1.0×10⁵/mL to 2.5×10⁵/mL cells (e.g., ED-I-derived cells) are contacted with the composition for ED II. As a non-limiting example, at least 1.0×10⁵/mL, at least 1.1×10⁵/mL, at least 1.2×10⁵/mL, at least 1.3×10⁵/mL, at least 1.4×10⁵/mL, at least 1.5×10⁵/mL, at least 1.6×10⁵/mL, at least 1.7×10⁵/mL, at least 1.8×10⁵/mL, at least 1.9×10⁵/mL, at least 2.0×10⁵/mL, at least 2.1×10⁵/mL, at least 2.2×10⁵/mL, at least 2.3×10⁵/mL, at least 2.4×10⁵/mL, or at least 2.5×10⁵/mL, or more cells (e.g., ED-I-derived cells) are contacted with the composition for ED II.

In one embodiment, 1.0×10⁶/mL to 5.0×10⁶/mL cells (e.g., ED-II-derived cells) are contacted with the composition for ED III. As a non-limiting example, at least 1.0×10⁶/mL, at least 1.1×10⁶/mL, at least 1.2×10⁶/mL, at least 1.3×10⁶/mL, at least 1.4×10⁶/mL, at least 1.5×10⁶/mL, at least 1.6×10⁶/mL, at least 1.7×10⁶/mL, at least 1.8×10⁶/mL, at least 1.9×10⁶/mL, at least 2.0×10⁶/mL, at least 2.1×10⁶/mL, at least 2.2×10⁶/mL, at least 2.3×10⁶/mL, at least 2.4×10⁶/mL, at least 2.5×10⁶/mL, at least 3.0×10⁶/mL, at least 3.5×10⁶/mL, at least 4.0×10⁶/mL, at least 4.5×10⁶/mL, at least 5.0×10⁶/mL, or more cells (e.g., ED-II-derived cells) are contacted with the composition for ED III.

In another aspect, described herein is a method comprising: (a) differentiating a population of pluripotent stem cells into a population of HSCs or HSPCs; and (b) differentiating the resultant population of HSCs or HSPCs in a composition described herein to promote induction into a RBC or population thereof.

### Pluripotent Stem Cells

In some embodiments of any of the aspects, the methods described herein comprises differentiating a population of pluripotent stem cells. Pluripotent stem cells (PSCs) have the potential to give rise to all the somatic tissues. In one embodiment of any method, cells, or composition described herein, the population of pluripotent stem cells is induced pluripotent stem cells (iPSCs) or embryonic stem cells (ESC). IPSC and ESC can be produced by any method known in the art. In some embodiments of any of the aspects, the population of pluripotent stem cells comprises embryonic stem cells (ESC). Embryonic stem cells (ESCs) are stem cells derived from the undifferentiated inner mass cells of a human embryo.

In one embodiment, any of the methods for inducing a RBC described herein further comprises, prior to contacting, the step of obtaining a stem cell source.

In one embodiment, any of the methods for inducing a RBC described herein further comprises, prior to contacting, the step of generating an iPSCs, e.g., from a somatic cell.

Directed differentiation of PSCs aims to recapitulate embryonic development to generate patient-matched tissues by specifying the three germ layers. A common theme in directed differentiation across all germ layers is the propensity of PSCs to give rise to embryonic- and fetal-like cell types, which poses a problem for integration and function in an adult recipient. This distinction is particularly striking in the hematopoietic system, which emerges in temporally and spatially separated waves at during ontogeny. The earliest “primitive” progenitors emerge in the yolk sac at 8.5 dpc and give rise to a limited repertoire of macrophages, megakaryocytes and nucleated erythrocytes. These early embryonic-like progenitors are generally myeloid-based and cannot functionally repopulate the bone marrow of adult recipients. By contrast, “definitive” cells with hematopoietic stem cell (HSC) potential emerge later in arterial endothelium within the aorta-gonad-mesonephros (AGM) and other anatomical sites. Directed differentiation of PSCs gives rise to hematopoietic progenitors, which resemble those found in the yolk sac of the early embryo. These lack functional reconstitution potential, are biased to myeloid lineages, and express embryonic globins. Thus, understanding key fate determining mechanisms that promote development of either primitive or definitive lineages is critical for specifying HSCs, and other adult-like cell types (e.g., red blood cells) from PSCs.

In some embodiments of any of the aspects, the population of pluripotent stem cells (PSCs) comprises induced pluripotent stem cells (iPS cells). In some embodiments of any of the aspects, the induced pluripotent stem cells are produced by introducing only reprogramming factors OCT4, SOX2, KLF4 and optionally c-MYC or nanog and LIN28 into mature cells. In some embodiments of any of the aspects, the induced pluripotent stem cells are produced by introducing the reprogramming factors two or more times into the mature cells.

In some embodiments, the pluripotent stem cells (PSCs) described herein are induced pluripotent stem cells (iPSCs). An advantage of using iPSCs is that the cells can be derived from the same subject to which the eventual RBC would be reintroduced. That is, a somatic cell can be obtained from a subject, reprogrammed to an induced pluripotent stem cell, and then transfected and differentiated into a modified RBC to be administered to the subject (e.g., autologous cells). Since the progenitors are essentially derived from an autologous source, the risk of engraftment rejection or allergic responses is reduced compared to the use of cells from another subject or group of subjects. In some embodiments, the cells for generating iPSCs are derived from non-autologous sources. In addition, the use of iPSCs negates the need for cells obtained from an embryonic source. Thus, in one embodiment, the PSCs used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a common myeloid stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein.

Reprogramming methodologies for generating pluripotent cells using defined combinations of transcription factors have been described to induce pluripotent stem cells from somatic cells. Yamanaka and Takahashi converted mouse somatic cells to ES cell-like cells with expanded developmental potential by the direct transduction of Oct4, Sox2, Klf4, and optionally c-Myc. See, e.g., U.S. Pat. Nos. 8,058,065 and 9,045,738 to Yamanaka and Takahashi, which are incorporated herein by reference. iPSCs resemble ES cells as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission, and tetraploid complementation.

Subsequent studies have shown that human iPS cells can be obtained using similar transduction methods, and the transcription factor trio, OCT4, SOX2, and NANOG, has been established as the core set of transcription factors that govern pluripotency. The production of iPS cells can be achieved by the introduction of nucleic acid sequences encoding stem cell-associated genes into an adult, somatic cell, using viral vectors.

OCT4, SOX2, KLF4 and c-MYC are the original four transcription factors identified to reprogram mouse fibroblasts into iPSCs. These same four factors were also sufficient to generate human iPSCs. OCT3/4 and SOX2 function as core transcription factors of the pluripotency network by regulating the expression of pluripotency-associated genes. Kruppel-like factor 4 (KLF4) is a downstream target of LIF-STAT3 signaling in mouse ES cells and regulates self-renewal. Human iPSCs can also be generated using four alternative factors; OCT4 and SOX2 are required but KLF4 and c-MYC could be replaced with NANOG, a homeobox protein important for the maintenance of pluripotency in both ES cells and early embryos, and LIN28, an RNA binding protein. The combination of OCT4, SOX2, NANOG and LIN28 reprogramming factors have been reported to be also sufficient to generate human iPSCs.

In one embodiment of any method, cells, or composition described herein, the iPSCs are produced, for example, by introducing exogenous copies of only three reprogramming factors OCT4, SOX2, and KLF4 into mature or somatic cells. In one embodiment of any method, cells, or composition described herein, c-MYC, or nanog and/or LIN28 are further introduced to iPSCs having exogenous gene coding copies of OCT4, SOX2, and KLF4 to differentiate into mature or somatic cells. In one embodiment of any method, cells, or composition described herein, the iPSCs are produced by introducing exogenous copies of reprogramming factors OCT4, SOX2, and KLF4, and optionally with c-MYC or nanog and/or LIN28 to differentiate into mature or somatic cells.

In one embodiment of any method, cells, or composition described herein, the iPSCs are produced by contacting mature cells with at least one vector, wherein the at least one vector carries an exogenous gene coding copy of reprogramming factors OCT4, SOX2, and KLF4, and optionally with c-MYC, or nanog and/or LIN28 to differentiate into mature or somatic cells, and wherein the reprogramming factors are expressed in vivo in the contacted mature or somatic cells. The contacting is in vitro or ex vivo. The reprogramming factors needed for differentiation can all be expressed by one vector (e.g., a vector that carries an exogenous gene coding copy of OCT4, SOX2, KLF4, and c-MYC). Alternatively, the reprogramming factors can be expressed in more than one vector that is each used to contact the iPSCs. For example, an iPSCs can be contacted by a first vector that carries an exogenous gene coding copy of OCT4, SOX2, and a second vector that carries an exogenous gene coding copy KLF4 and c-MYC.

In one embodiment of any disclosed methods, the iPS cell comprises at least an exogenous copy of a nucleic acid sequence encoding a reprogramming factor selected from the group consisting of genes Oct4 (Pou5f1), Sox2, cMyc, Klf4, Nanog, Lin 28 and Glis1. In some embodiments, combinations of reprogramming factors are used. For example, a combination of four reprogramming factors consisting of Oct4, Sox2, cMyc, and Klf4, or a combination of four reprogramming factors consisting of Oct4, Sox2, Nanog, and Lin 28.

In one embodiment of any method, cells, or composition described herein, the iPSCs are produced by introducing the disclosed reprogramming factors, or any combination of the reprograming factors two or more times into the mature or somatic cells. In one embodiment, the combination of reprograming factors is different when a combination is introduced to the iPSC more than once, for example, the combination of Oct4 (Pou5f1), Sox2, cMyc, Klf4, Nanog is first introduced to the iPSCs, and the combination of Oct4 (Pou5f1), Sox2, cMyc is subsequently introduced to the iPSCs. In one embodiment of any method, cells, or composition described herein, the iPSCs are produced by contacting mature cells with the disclosed vector(s) factors two or more times into the mature/somatic cells.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5; 7(5):618-30, this reference is incorporated herein by reference in its entirety). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135, the contents of each of which are incorporated herein by reference in its entirety. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-CI-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, FUKASAWA, MERCK BIOSCIENCES, NOVARTIS, GLOUCESTER PHARMACEUTICALS, ATON PHARMA, TITAN PHARMACEUTICALS, SCHERING AG, PHARMION, METHYLGENE, and SIGMA ALDRICH.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Daxl, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Many US Patents and Patent Application Publications teach and describe methods of generating iPSCs and related subject matter. For examples, U.S. Pat. Nos. 8,058,065, 9,347,044, 9,347,042, 9,347,045, 9,340,775, 9,341,625, 9,340,772, 9,250,230, 9,132,152, 9,045,738, 9,005,975, 9,005,976, 8,927,277, 8,993,329, 8,900,871, 8,852,941, 8,802,438, 8,691,574, 8,735,150, 8,765,470, 8,058,065, 8,048,675, and US Patent Publication Nos: 20090227032, 20100210014, 20110250692, 20110201110, 20110200568, 20110223669, 20110306516, 20100021437, 20110256626, 20110044961, 20120276070, 20120214243, 20120263689, 20120128655, 20120100568, 20130295064, 20130029866, 20130059386, 20130183759, 20130189786, 20130295579, 20130130387, 20130157365, 20140234973, 20140227736, 20140093486, 20140301988, 20140170746, 20140178989, 20140349401, 20140065227, and 20150140662, all of which are incorporated herein by reference in their entireties.

In some embodiments of any of the aspects, the iPSCs can be derived from somatic cells. Somatic cells, as that term is used herein, refer to any cells forming the body of an organism, excluding germline cells. Every cell type in the mammalian body-apart from the sperm and ova, the cells from which they are made (gametocytes) and undifferentiated stem cells-is a differentiated somatic cell. For example, internal organs, skin, bones, blood, and connective tissue are all made up of differentiated somatic cells. In one embodiment of any method, cells, or composition described herein, the mature cells from which iPS cells are made include any somatic cells such as B lymphocytes (B-cells), T lymphocytes, (T-cells), and fibroblasts and keratinocytes.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, skin, immune cells, hepatic, splenic, lung, peripheral circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; beta-III-tubulin; alpha-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived. In one embodiment, the iPSCs are derived from mature, differentiated, somatic cells.

In some embodiments of any of the aspects, the population of pluripotent stem cells used in the differentiation methods described herein does not comprise CD34+ HSPCs or multipotent lymphoid progenitors (MLPs) purified from a patient sample. In some embodiments of any of the aspects, the population of pluripotent stem cells does not comprise stem cells purified or isolated from cord blood or bone marrow samples. In some embodiments of any of the aspects, the population of pluripotent stem cells is not derived from stem cells isolated from a patient sample (e.g., cord blood or bone marrow). In a preferred embodiment, the population of pluripotent stem cells comprise iPSCs, such as those derived from a somatic cell sample from a patient. See e.g., Tabatabaei-Zavareh et al., J Immunol May 1, 2017, 198 (1 Supplement) 202.9.

### Hematopoietic Stem Cells

In some embodiments of any of the aspects, the methods described herein comprises differentiating a population of HSCs. HSCs are known to give rise to committed hematopoietic progenitor cells (HPCs) that are capable of generating the entire repertoire of mature blood cells over the lifetime of an organism. The term “hematopoietic stem cell” or “HSC” generally refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Patent No. 5,750,397; Schwartz, et al., U.S. Pat. No. 5,759,793; DiGuisto, et al., U.S. Patent No. 5,681,599; Tsukamoto, et al., U.S. Pat. No. 5,716,827; the contents of which are incorporated herein by reference in their entireties). When transplanted into lethally irradiated animals or humans, hematopoietic stem and progenitor cells can repopulate the erythroid, neutrophil-macrophage, megakaryocyte and lymphoid hematopoietic cell pool.

In one embodiment, any of the methods for inducing a RBC described herein further comprises, prior to contacting, the step of obtaining a HSC or HSPC.

In one embodiment, any of the methods for inducing a RBC described herein further comprises, prior to contacting, the step of differentiating a stem cell, e.g., an iPSC into a HSC or HSPC.

In one embodiment, the term “hematopoietic stem cell” or “HSC” refers to a stem cell that have the following cell surface markers: CD34⁺, CD59⁺, Thy1/CD90⁺, CD38^{lo/-}, and C-kit/CD117⁺. In one embodiment, the term “hematopoietic stem cell” or “HSC” refers to a stem cell that is at least CD34⁺. In one embodiment, the term “hematopoietic stem cell” or “HSC” refers to a stem cell that is at least CD38lo/⁻. In one embodiment, the term “hematopoietic stem cell” or “HSC” refers to a stem cell that is at least CD34⁺ and CD38^{lo/-}. In one embodiment, the term “hematopoietic stem cell” or “HSC” refers to a stem cell that is at least lin⁻. In one embodiment, the term “hematopoietic stem cell” or “HSC” refers to a stem cell that is at least CD34⁺ and lin⁻. In one embodiment, the term “hematopoietic stem cell” or “HSC” refers to a stem cell that is at least CD34⁺, CD38^{lo/-} and lin⁻. In one embodiment, the term “hematopoietic stem cell” or “HSC” refers to a stem cell that is at least CD34⁺ and C-kit/CD117⁺. In one embodiment, the term “hematopoietic stem cell” or “HSC” refers to a stem cell that is at least CD34⁺, CD38^{lo/-} and C-kit/CD117⁺. In another embodiment, as used herein, the term “hematopoietic stem cell” or “HSC” includes hematopoietic stem and progenitor cells (HSPC).

Mature blood cells have a finite lifespan and must be continuously replaced throughout life. Blood cells are produced by the proliferation and differentiation of a very small population of pluripotent HSCs that also have the ability to replenish themselves by self-renewal. HSCs are multipotent, self-renewing progenitor cells that develop from mesodermal hemangioblast cells. HSCs are the blood cells that give rise to all the other blood cells, that includes all the differentiated blood cells from the erythroid, lymphoid and myeloid lineages. HSCs are located in the adult bone marrow, peripheral blood, and umbilical cord blood.

During differentiation, the progeny of HSCs progress through various intermediate maturational stages, generating multi-potential hematopoietic progenitor cells and lineage-committed hematopoietic progenitor cells, prior to reaching maturity. Bone marrow (BM) is the major site of hematopoiesis in humans and, under normal conditions, only small numbers of HSCs and hematopoietic progenitor cells can be found in the peripheral blood (PB). Treatment with cytokines (in particular granulocyte colony-stimulating factor; G-CSF), myelosuppressive drugs used in cancer treatment, and compounds that disrupt the interaction between hematopoietic cells and BM stromal cells can rapidly mobilize large numbers of stem and progenitor cells into the circulation.

The HSCs, similar to the hematopoietic progenitor cells, are capable of proliferation and giving rise to more progenitor cells having the ability to generate a large number of mother cells that can in turn give rise to differentiated or differentiable daughter cells. The daughter cells themselves can be stimulated to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In one embodiment, HSCs are obtained or isolated from bone marrow, umbilical cord, chorionic villi, amniotic fluid, placental blood, cord blood or peripheral blood. One skilled in the art will know how to obtain or isolate such cell population using standard protocol known in the art and/or described herein. Methods of mobilizing HSCs from the places of origin or storage are known in the art. For example, treatment with cytokines, in particular granulocyte colony-stimulating factor (G-CSF) and compounds (e.g., plerixafor, a chemokine CXCR4 antagonist) that disrupt the interaction between HSCs and bone marrow (BM) stromal cells can rapidly mobilize large numbers of hematopoietic stem and hematopoietic progenitor cells into the circulation. In one embodiment, CD34⁺ HSCs are obtained or isolated from the bone marrow, umbilical cord, chorionic villi, amniotic fluid, placental blood, cord blood or peripheral blood.

### Compositions Comprising Induced Blood Cells

One aspect provided herein is an induced red blood cell, e.g., an iRBC, or population thereof derived using any of the methods described herein. An iRBC is a mature, and is enucleated, i.e., lacking a nucleus. iRBCs express at least one cellular marker selected from GlyA (CD235a), Band 3, and CD71. One skilled in the art can determine if an iRBC expresses RBC expresses at least one cellular marker selected from GlyA (CD235a), Band 3, or CD71 via standard techniques, such as FACS sorting or immunohistochemistry.

In one embodiment, at least 75% of the iRBCs in the composition are positive for at least one cellular marker selected from GlyA (CD235a), Band 3, or CD71. As a non-limiting example, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% of the iRBCs in the composition are positive for at least one cellular marker selected from GlyA (CD235a), Band 3, or CD71. One skilled in the art can assess whether an iRBC, or population thereof is positive for at least cellular marker using standard techniques. For example, a skilled person can utilize flow cytometry and/or immunofluorescence techniques with commercially available antibodies specific to the cellular markers to visualize the cellular marker. Alternatively, mRNA or protein levels of the cellular marker can be detected in an iRBC using PCR-based assays or Western blotting, respectively.

In one embodiment, at least 75% of the iRBCs in the composition are enucleated, i.e., negative for DNA. As a non-limiting example, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% of the iRBCs in the composition are enucleated, i.e., negative for DNA. One skilled in the art can assess whether an iRBC, or population thereof is enucleated using standard techniques. For example, a skilled person can, e.g., utilize flow cytometry and/or immunofluorescence techniques with commercially available antibodies specific to DNA (e.g., chromatin) to visualize the DNA.

Another aspect provided herein is a composition comprising the population of induced blood cells, e.g., iRBC, of the invention. In one embodiment, the composition of induced blood cells further comprises a pharmaceutically acceptable carrier.

Yet another aspect provided herein is a pharmaceutical composition comprising the population of induced blood cells, e.g., iRBC, of the invention and a pharmaceutically acceptable carrier.

In general, the iRBCs as described herein are administered as a suspension with a pharmaceutically acceptable carrier. For example, as pharmaceutical compositions. Pharmaceutical compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells as described herein using routine experimentation.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

In one embodiment, the “pharmaceutically acceptable” carrier does not include in vitro cell culture media.

### Methods of Treatment

Provided herein are methods of treating a subject in need of a blood transfusion comprising administering any of the induced blood cells, e.g., iRBCs or population thereof, or compositions comprising iRBCs described herein to a recipient subject in need thereof.

Further provided herein are methods of treating a blood disease or disorder, e.g., sickle cell anemia, a subject in need thereof comprising administering any of the iRBC or population thereof, or compositions comprising iRBCs described herein to a recipient subject in need thereof

In various embodiments, the methods further comprise, prior to administering, the step of diagnosing a subject as having a blood disease or disorder. In various embodiments, the methods further comprise, prior to administering, the step of receiving the results of an assay that diagnoses a subject as having a blood disease or disorder. A skilled clinician can diagnose a subject as having a blood disease or disorder using standard tests or procedures, e.g., a complete blood count (CBC) to assess blood cell levels and health (e.g., shape, size, etc.).

In various embodiments, the methods further comprise, prior to administering, the step of diagnosing a subject as needing a blood transfusion. For example, a subject having a disease or disorder that inhibits proper RBC formation or production, a bacterial or viral infection, an injury resulting in a loss of blood, or a surgery resulting in a loss of blood may be in need of a transfusion. In various embodiments, the methods further comprise, prior to administering, the step of receiving the results of an assay that diagnoses a subject as needing a blood transfusion. A skilled clinician can diagnose a subject as needing a blood transfusion using standard tests or procedures, e.g., measuring hemoglobin levels. For example, a subject is likely in need of a blood transfusion if the subject's hemoglobin levels are below 10 g/dL, 9 g/dL, 8 g/dL, or 7 g/dL.

Another aspect described herein provides a method of treating a disease or disorder that inhibits proper RBC formation or production, the method comprising administering any induced RBC or population thereof described herein, or any composition of iRBCs described herein, or any pharmaceutical composition of iRBCs described herein to a recipient subject diagnosed as having a disease or disorder that inhibits proper RBC formation or production. Exemplary diseases or disorders that inhibit proper RBC formation or production include but are not limited to hemoglobinopathies (congenital abnormality of the hemoglobin molecule or of the rate of hemoglobin synthesis), examples of which include sickle-cell disease, thalassemia, and methemoglobinemia; Anemias (lack of red blood cells or hemoglobin), Pernicious anemia; disorders resulting in decreased numbers of cells, such as myelodysplastic syndrome, neutropenia (decrease in the number of neutrophils), and thrombotic thrombocytopenic purpura (TTP), thrombocytosis, hematological malignancies such as lymphomas, myelomas, and leukemia; Lymphomas such as Hodgkin's disease, Non-Hodgkin's lymphoma, Burkitt's lymphoma, Anaplastic large cell lymphoma, Splenic marginal zone lymphoma, Hepatosplenic T-cell lymphoma, and Angioimmunoblastic T-cell lymphoma (AILT); myelomas such as Multiple myeloma, Waldenstrom macroglobulinemia, Plasmacytoma; leukemias that increases defect WBC such as Acute lymphocytic leukemia (ALL), Chronic lymphocytic leukemia (CLL), Acute myelogenous leukemia (AML), Chronic Idiopathic Myelofibrosis (MF), Chronic myelogenous leukemia (CML), T-cell prolymphocytic leukemia (T-PLL), B-cell prolymphocytic leukemia (B-PLL), Chronic neutrophilic leukemia (CNL), Hairy cell leukemia (HCL), T-cell large granular lymphocyte leukemia (T-LGL), and Aggressive NK-cell leukemia. A skilled clinician can diagnose a having a disease or disorder that inhibits proper RBC formation or production using standard tests or procedures, e.g., a CBC, assessment of known symptoms, family history, and genetic sequencing for known mutations.

One aspect herein is a method of providing a blood transfusion, or for the treatment of that inhibit proper RBC formation or production in a subject, comprising (a) providing a somatic cell from a donor subject, (b) generating an iPSCs from the somatic cell of (a), (c) inducing the differentiation of a RBC using any of the methods described herein, and (d) administering the resultant differentiated RBCs into a recipient subject.

One aspect herein is a method of providing a blood transfusion, or for the treatment of that inhibit proper RBC formation or production in a subject, comprising (a) providing a somatic cell from a subject, (b) generating an iPSCs from the somatic cell of (a), (c) inducing the differentiation of a RBC using any of the methods described herein, and (d) administering the resultant differentiated RBCs into the subject.

One aspect herein is a method of providing a blood transfusion, or for the treatment of that inhibit proper RBC formation or production in a subject, comprising (a) providing a somatic cell from a donor subject, (b) generating an iPSC from the somatic cell of (a), (c) differentiating the iPSC of (b) into a HSC or HSPC, and (d) inducing the differentiation of a RBC using any of the methods described herein, and (d) administering the resultant differentiated RBCs into a recipient subject.

One aspect herein is a method of providing a blood transfusion, or for the treatment of that inhibit proper RBC formation or production in a subject, comprising (a) providing a somatic cell from a subject, (b) generating an iPSC from the somatic cell of (a), (c) differentiating the iPSC of (b) into a HSC or HSPC, and (d) inducing the differentiation of a RBC using any of the methods described herein, and (d) administering the resultant differentiated RBCs into the subject.

One aspect herein is a method of providing a blood transfusion, or for the treatment of that inhibit proper RBC formation or production in a subject, comprising (a) providing an iPSC, (b) inducing the differentiation of a RBC using any of the methods described herein, and (c) administering the resultant differentiated RBCs into a recipient subject.

One aspect herein is a method of providing a blood transfusion, or for the treatment of that inhibit proper RBC formation or production in a subject, comprising (a) providing an iPSC, (b) differentiating the iPSC of (a) into a HSC or HSPC, and (c) inducing the differentiation of a RBC using any of the methods described herein, and (d) administering the resultant differentiated RBCs into a recipient subject.

In one embodiment, the host subject and the recipient subject are the same individual. Alternatively, the host subject and the recipient subject are not the same individual, but are at least HLA compatible.

As used herein, the terms “administering,” “introducing” and “transplanting” are used interchangeably in the context of the placement of described cells, e.g. iRBC, into a subject, by a method or route which results in at least partial localization of the introduced cells at a desired site, such as a site of injury or repair, such that a desired effect(s) is produced. The cells, e.g., iRBCs can be administered by any appropriate route which results in delivery to a desired location in the subject where at least a portion of the administered cells or components of the cells remain viable.

In one embodiment, prior to administering, the iRBCs is engineered to modify or manipulate its genome. In one embodiment, the stem cell source, e.g., an iPSC or HSC, can be engineered to modify or manipulate its genome, such that RBCs induced therefrom have the modified or manipulated genome.

The engineered iRBCs described herein are useful in the laboratory for biological studies. For examples, these cells can be derived from an individual having a genetic disease or defect, and used in the laboratory to study the biological aspects of the disease or defect, and to screen and test for potential remedy for that disease or defect.

Alternatively, the engineered iRBCs described herein are useful in therapy and other medical treatment in subjects having the need, for example, patients having a blood disorder such as sickle cell anemia. Disease-causing mutations for sickle cell anemia can be corrected in iRBCs derived from the patient's own stem cells, thus the patient can be administered autologous RBCs that are engineered to be wild-type (e.g., disease free).

One skilled in the art can modify the genome of a cell, e.g., an iRBC or stem cell source described herein, using any technique known in the art. For example, genome editing systems including, but not limited to, zinc finger nucleases, TALENS, meganucleases, and CRISPR/Cas systems, can be used to engineer a cell's genome. In one embodiment, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference. The CRISPR/Cas system is originally an RNA-mediated bacterial immune system that provides a form of acquired immunity against viruses and plasmids; it comprises three components: a Cas (CRISPR associated protein) endonuclease (such as Streptococcus pyogenes Cas9 or Francisella novicida Cas12a), a crRNA (CRISPR RNA), and a tracrRNA (transactivating crRNA). Clustered regularly interspaced short palindromic repeats (CRISPR) are short repetitions of bacterial DNA followed by short repetitions of spacer DNA from viruses or plasmids. The Cas9 endonuclease contains two nuclease domains and is programmed by a crRNA and tracrRNA hybrid to cleave the target sequence. In some embodiments, the Cas9 endonuclease is programmed by a crRNA and tracrRNA hybrid to cleave, e.g., a Notch4 sequence. In other embodiments, the Cas9 endonuclease is programmed by a single-guide RNA (sgRNA), which contains both a crRNA and tracrRNA sequence. In some cases, the guide RNAs (gRNAs) are selected to generate a functional gene deletion, in other cases the gRNAs are selected to recruit a catalytically inactive Cas molecule to inhibit transcription of the target loci (CRISPR interference; CRISPRi) or activate transcription of human target loci (CRISPR activation; CRISPRa).

There are two main considerations in the selection of the 20-nt guide sequence for gene targeting: 1) the target sequence should precede the protospacer adjacent motif (PAM) sequence specific for the Cas nucleus used (5′-GG PAM for S. pyogenes Cas9), and 2) guide sequences should be chosen to minimize off-target activity. Guide RNA sequences can be readily generated for a given target sequence using prediction software, for example, CRISPRdirect (available on the world wide web at crispr.dbels.jp/), see Natio, et al. Bioinformatics. (2015) April 1; 31(7): 1120-1123; ATUM gRNA Design Tool (available on the world wide web at atum.bio:ecommerce/cas9/input); an CRISPR-ERA (available on the world wide web at crispr-era.stanford.eduu/indexjsp), see Liu, et al. Bioinformatics, (2015) November 15; 31(22): 3676-3678. All references cited herein are incorporated herein by reference in their entireties. Non-limiting examples of publicly available gRNA design software include; sgRNA Scorer 1.0, Quilt Universal guide RNA designer, Cas-OFFinder & Cas-Designer, CRISPR-ERA, CRISPR/Cas9 target online predictor, Off-Spotter—for designing gRNAs, CRISPR MultiTargeter, ZiFiT Targeter, CRISPRdirect, CRISPR design from crispr.mit.edu/, E-CRISP etc.

A CRISPR/Cas system can be delivered using a plasmid, vector, or a ribonucleoprotein complex. Ribonucleoprotein complexes comprising a Cas protein can further comprise a nucleic acid sequence encoding crRNA and tracrRNA. When a nucleic acid encoding one or more sgRNAs and a nucleic acid encoding an RNA-guided endonuclease each need to be administered, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

Alternatively, a cell can be engineered via contact with inhibitors of the expression of a given gene, for example, an inhibitory nucleic acid, such as an inhibitory RNA (iRNA). The RNAi can be single stranded or double stranded.

The iRNA can be siRNA, shRNA, endogenous microRNA (miRNA), or artificial miRNA. In one embodiment, an iRNA as described herein effects inhibition of the expression and/or activity of a target, e.g. Notch4. In some embodiments of any of the aspects, the agent is siRNA that inhibits Notch4. In some embodiments of any of the aspects, the agent is shRNA that inhibits Notch4.

One skilled in the art would be able to design siRNA, shRNA, or miRNA to target Notch4, e.g., using publically available design tools. siRNA, shRNA, or miRNA is commonly made using algorithms, such as RNAi Design (available, e.g., on the world wide web at maidesigner.thermofisher.com/rnaiexpress/ and on the world wide web at biotools.idtdna.com/site/order/designtool/index/DSIRNA_CUSTOM); DHARMACON (Lafayette, CO) (available, e.g., on the world wide web at https://www.thermofisher.com/order/custom-genomic-products/tools/sirna/); or SIGMA ALDRICH (St. Louis, Mo.) (available, e.g., on the world wide web at sigmaaldrich.com/life-science/custom-oligos/sirna-oligos/sirna-design-service.html).

In various embodiments, the engineered cells described herein are optionally expanded ex vivo prior to administration to a subject. In other embodiments, the engineered RBCs are optionally cryopreserved for a period, then thawed prior to administration to a subject.

In various embodiments, the iRBCs described herein are administered (i.e., implanted or transplanted) to a subject in need of a blood transfusion.

The iRBCs used for therapy can be autologous/autogenic (“self”) or non-autologous (“non-self,” e.g., allogeneic, syngeneic or xenogeneic) in relation to the recipient of the cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells of the invention are allogeneic.

In various embodiments, the iRBCs described herein can be derived from one or more donors, or can be obtained from an autologous source. In some embodiments, the iRBCs expanded in culture prior to administration to a subject in need thereof.

In various embodiments, the recipient subject is a human.

In one embodiment, a subject is selected to donate a somatic cell which would be used to produce iPSCs and an iRBCs described herein. In one embodiment, the selected subject has a genetic disease or defect.

In various embodiments, the donor subject is a human, non-human animal, rodent or non-rodent. For example, the subject can be any mammal, e.g., a human, other primate, pig, rodent such as mouse or rat, rabbit, guinea pig, hamster, cow, horse, cat, dog, sheep or goat, or a non-mammal such as a bird.

In various embodiments, the donor has been previously diagnosed with a hematological disease or disorder.

In one embodiment, a biological sample, a population of embryonic stem cells, somatic stem cells, progenitor cells, bone marrow cells, hematopoietic stem cells, or hematopoietic progenitor cells is obtained from the donor subject.

In various embodiments, the biological sample, a population of embryonic stem cells, somatic stem cells, progenitor cells, bone marrow cells, hematopoietic stem cells, or hematopoietic progenitor cells described herein can be derived from one or more donors, or can be obtained from an autologous source.

In another embodiment, the embryonic stem cells, somatic stem cells, progenitor cells, bone marrow cells, hematopoietic stem cells, or hematopoietic progenitor cells are isolated from a donor who is an HLA-type match with a subject (recipient). Donor-recipient antigen type-matching is well known in the art. The HLA-types include HLA-A, HLA-B, HLA-C, and HLA-D. These represent the minimum number of cell surface antigen matching required for transplantation. One added advantage is that if the donor of the source cells and recipient of the iRBCs are the same person, the produced iRBCs have HLA that are identical to the recipient and this avoids host-graft immune rejection after the transplantation. For recipient patients that are HLA allogeneic to the donor person of the source cells, host-graft immune rejection is greatly reduced.

In one embodiment, a dose of cells is delivered to a subject intravenously. In one embodiment, the cells are intravenously administered to a subject.

In particular embodiments, subjects receive a dose of cells described herein, e.g., iRBCs, of about 1×10⁵ cells/kg, about 5×10⁵ cells/kg, about 1×10⁶ cells/kg, about 2×10⁶ cells/kg, about 3 x 10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10′ cells/kg, about 5 x cells/kg, about 1×10⁸ cells/kg, or more in one single intravenous dose. In certain embodiments, patients receive a dose of genetically modified cells, e.g., iRBCs, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10′ cells/kg, at least 5×10′ cells/kg, at least 1×10⁸ cells/kg, or more in one single intravenous dose.

In an additional embodiment, subjects receive a dose of cells described herein, e.g., iRBCs, of about 1×10⁵ cells/kg to about 1×10⁸ cells/kg, about 1×10⁶ cells/kg to about 1×10⁸ cells/kg, about 1×10⁶ cells/kg to about 9×10⁶ cells/kg, about 2×10⁶ cells/kg to about 8×10⁶ cells/kg, about 2×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, at least a second or subsequent dose of cells is administered to the recipient subject. For example, a second administration can be given between about one day to 30 weeks from the previous administration. Two, three, four or more total subsequent administrations can be delivered to the individual, as needed, e.g., determined by a skilled clinician.

A cell composition can be administered by any appropriate route which results in effective cellular replacement treatment in the subject, i.e. administration results in delivery to a desired location in the subject where at least a portion of the composition delivered, i.e. at least 1×10⁴ cells are delivered to the desired site for a period of time. Modes of administration include injection, infusion, or instillation. “Injection” includes, without limitation, intravenous, intra-arterial, intraventricular, intracardiac injection and infusion. For the delivery of cells, administration by injection or infusion is generally preferred.

Efficacy testing can be performed during the course of treatment using the methods described herein. Measurements of the degree of severity of a number of symptoms associated with a particular ailment are noted prior to the start of a treatment and then at later specific time period after the start of the treatment. In some embodiments of any of the aspects, a pharmaceutical composition comprising an immune as described herein or a population thereof can be used for a therapy in a subject.

Accordingly, it is also the objective of this the present disclosure to provide compositions of modified (also referred to as engineered) cells for use in in vivo medical therapy such as blood disorder therapy, and for the in vitro studies of disease modeling, drug screening, and hematological diseases.

In one embodiment, any of the methods described herein further comprise administering at least one additional therapeutic, e.g., a drug and agent known in the art for the treatment of a disease or disorder that that inhibits proper RBC formation or production, or for the treatment of a subject in need of a blood transfusion. A non-limiting example of a second agent and/or treatment can include iron supplements. A skilled clinician can determine the correct additional therapeutic to administer to an individual having a given disease or disorder.

### Kits

Another aspect of the technology described herein relates to kits for differentiating RBCs, using compositions and method as described herein, among others. Described herein are kit components that can be included in one or more of the kits described herein.

Another aspect described herein provides a kit for inducing a RBC comprising: (a) a first composition comprising at least one of the components selected from the group consisting of: human AB plasma, an antibiotic, heparin, insulin, human holo-transferrin, a corticosteroid, and polyvinyl alcohol (PVA); (b) a second composition comprising IL-3, SCF, and EPO; (c) a third composition comprising SCF and EPO; and (d) a fourth composition comprising EPO and optionally human holo-transferrin.

In some embodiments, the kit comprises an effective amount of components of the compositions described herein; and an effective amount of differentiation factors (e.g., IL-3, SCF, and/or EPO); or an effective amount of iPSC differentiation factors (e.g., OCT4, SOX2, KLF4, c-MYC, nanog, and/or LIN28); or an effective amount of hemogenic differentiation factors (e.g., BMP4, SB-431542, CHIR99021, bFGF, VEGF, IL-6, IL-11, IGF-1, SCF, and EPO). In one embodiment, a kit can comprise any of the compositions or differentiation factors described herein, e.g., the kit can comprise an effective amount of components of the compositions described herein and an effective amount of differentiation factors. As will be appreciated by one of skill in the art, such cell differentiation factors can be supplied in a lyophilized form or a concentrated form that can diluted prior to use with cultured cells. Preferred formulations include those that are non-toxic to the cells and/or does not affect growth rate or viability etc. Components of the compositions described herein and/or differentiation factors can be supplied in aliquots or in unit doses.

In some embodiments, the kit comprises a stem cell source, e.g., a iPSCs or HSC. In some embodiments, the kit comprises a cell source that can be reprogrammed into a stem cell source, e.g., iPSCs or HSC. In some embodiments, the kit does not comprise a stem cell source or a cell source.

In some embodiments, the kit further comprises a means for genetically modifying a cell, e.g., a vector or gene editing tool.

In some embodiments, the components described herein can be provided singularly or in any combination as a kit. Such kits can optionally include one or more agents that permit the detection of markers for a blood cell, e.g., a RBC (e.g., GlyA (CD235a), Band 3, and CD71) or a set thereof. Such kits can optionally include one or more agents that permit the detection of markers for hemogenic cell (e.g., CD34, CD38, CD45, KDR, CD235, CD43, etc.). In addition, the kit optionally comprises informational material.

In some embodiments, the compositions in the kit can be provided in a watertight or gas tight container which in some embodiments is substantially free of other components of the kit. For example, a cell differentiation reagent can be supplied in more than one container, e.g., it can be supplied in a container having sufficient reagent for a predetermined number of differentiation assays, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about the induction of blood cells, e.g., RBC, the method as described herein; or the concentration, date of expiration, batch or production site information, and so forth of reagents used herein such as cell differentiation factors. In one embodiment, the informational material relates to methods for using or administering the components of the kit.

The kit can include a component for the detection of a marker for cell differentiation. In addition, the kit can include one or more antibodies that bind a cell marker, or primers for an RT-PCR or PCR reaction, e.g., a semi-quantitative or quantitative RT-PCR or PCR reaction. Such components can be used to assess the activation of cell maturation markers or the loss of undifferentiated or immature cell markers. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A composition for inducing a red blood cell (RBC) comprising:
   human AB plasma;
   heparin;
   insulin;
   holo-transferrin;
   a corticosteroid; and
   polyvinyl alcohol (PVA).
2. The composition of paragraph 1, further comprising at least one growth factor.
3. The composition of paragraph 2, wherein the at least one growth factor is selected from the group consisting of IL-3, stem cell factor (SCF), and erythropoietin (EPO).
4. The composition of paragraph 1, wherein the corticosteroid is dexamethasone.
5. The composition of any of paragraphs 1-4, further comprising Iscove's DMEM (IMDM).
6. The composition of any of paragraphs 1-5, further comprising at least one antibiotic.
7. The composition of paragraph 6, wherein the at least one antibiotic is Penicillin and Streptomycin.
8. The composition of any of paragraphs 1-7, wherein PVA is present at a 0.08% concentration.
9. The composition of any of paragraphs 1-8, wherein the composition does not comprise bovine serum albumin (BSA).
10. The composition of any of paragraphs 1-8, wherein each component of the composition is of pharmaceutical grade.
11. A composition for inducing a RBC comprising at least one of the components selected from the group consisting of: human AB plasma, an antibiotic, heparin, insulin, human holo-transferrin, a corticosteroid, and polyvinyl alcohol (PVA)
12. The composition of paragraph 11, further comprising IL-3, SCF, and EPO.
13. The composition of paragraph 11, further comprising SCF and EPO.
14. The composition of paragraph 11, further comprising EPO.
15. The composition of any of paragraphs 11-14, wherein the composition does not comprise bovine serum albumin (BSA).
16. The composition of any of paragraphs 11-15, wherein the PVA is present at a 0.08% concentration.
17. A method of inducing a red blood cell (RBC), the method comprising contacting a stem cell with the composition of any of paragraphs 1-16 for a time sufficient to induce a RBC.
18. The method of paragraph 17, wherein the RBC is an enucleated RBC.
19. The method of paragraph 17 and 18, wherein the RBC expresses at least one cellular marker selected from GlyA (CD235a), Band 3, and CD71.
20. The method of paragraph 17, wherein the time sufficient is at least 18 days.
21. The method of paragraph 17, wherein the stem cell is an induced pluripotent stem cell (iPSC).
22. The method of paragraph 17, wherein the stem cell is a hematopoietic stem cell (HSC) or hematopoietic stem and progenitor cell (HSPC).
23. The method of any of paragraphs 17-22, wherein contacting occurs on an ultra-low attachment culture dish.
24. The method of any of paragraphs 17-23, wherein contacting occurs at 37° C. with at least 20% 02.
25. The method of any of paragraphs 17-24, wherein the composition is replaced at least every 2 or 3 days.
26. A method of inducing a red blood cell (RBC), the method comprising
   a) contacting a population of stem cells with the composition of paragraph 12;
   b) contacting the population of a) with the composition of paragraph 13; and
   c) contacting the population of b) with the composition of paragraph 14.
27. The method of any of paragraphs 17-26, wherein contacting is in vitro or ex vivo.
28. The method of paragraph 27, wherein contacting is culturing.
29. A RBC produced by any of the methods of paragraphs 11-28.
30. The RBC of paragraph 29, wherein the RBC is an enucleated RBC.
31. A composition comprising the RBC of paragraphs 29 or 30, or population thereof
32. The composition of paragraph 31, further comprising a pharmaceutically acceptable carrier.
33. A pharmaceutical composition comprising the RBC of paragraphs 29 or 30, or population thereof, and a pharmaceutically acceptable carrier.
34. The pharmaceutical composition of paragraph 33 for use in a blood transfusion in a subject.
35. A method of treating a subject in need of a blood transfusion, the method comprising administering a RBC of paragraphs 29 or 30, or population thereof, or a composition of paragraphs 31-32, or a pharmaceutical composition of paragraphs 33-34 to a recipient subject in need thereof
36. The method of paragraph 35, wherein the subject in need thereof has a disease or disorder that inhibits proper RBC formation or production.
37. The method of paragraph 36, wherein the disease or disorder is selected from the group consisting of anemia, cancer, hemophilia, kidney disease, liver disease, severe microbial infection, sickle cell disease, and thrombocytopenia, a hemoglobinopathies, Diamond-Blackfan Anemia, iron deficiency, B12 deficiency, folate deficiency, dyserythropoietic anemias, hemolytic anemias, metabolic disorders, the porphyrias, autoimmune diseases.
38. The method of paragraph 35, further comprising, prior to administering, diagnosing a subject as having a disease or disorder that inhibits proper RBC formation or production.
39. The method of paragraph 35, further comprising, prior to administering, receiving the results of an assay that diagnoses a subject as having a disease or disorder that inhibits proper RBC formation or production.
40. The method of paragraph 35, wherein the subject in need thereof has a hemoglobin level below 10 g/dL, 9 g/dL, 8 g/dL, or below 7 g/dL.
41. The method of paragraph 35, further comprising, prior to administering, diagnosing a subject as having hemoglobin level below 10 g/dL, 9 g/dL, 8 g/dL, or below 7 g/dL.
42. The method of paragraph 35, further comprising, prior to administering, receiving the results of an assay that diagnoses a subject as having hemoglobin level below 10 g/dL, 9 g/dL, 8 g/dL, or below 7 g/dL.
43. A method of treating a disease or disorder that inhibits proper RBC formation or production, the method comprising administering a RBC of paragraphs 29 or 30, or population thereof, or a composition of paragraphs 31-32, or a pharmaceutical composition of paragraphs 33-34 to a recipient subject diagnosed as having a disease or disorder that inhibits proper RBC formation or production.

44. The method of paragraph 43, further comprising, prior to administering, diagnosing a subject as having a disease or disorder that inhibits proper RBC formation or production.
45. The method of paragraph 43, further comprising, prior to administering, receiving the results of an assay that diagnoses a subject as having a disease or disorder that inhibits proper RBC formation or production.
46. The method of any of paragraphs 35-45, wherein the RBC is autologous or allogenic to the subject.
47. A method of transfusing a population of autologous RBCs to a subject in need thereof, the method comprising
   a) obtaining a stem cell source from a subject;
   b) inducing a population of RBCs according to the method of any of paragraphs 17-28; and
   c) administering the induced RBC population of b) via transfusion to the subject.
48. A method of treating a disease or disorder that inhibits proper RBC formation or production, the method comprising
   a) obtaining a stem cell source from a subject;
   b) inducing a population of RBCs according to the method of any of paragraphs 17-28; and
   c) administering the induced RBC population of b) via transfusion to the subject diagnosed as having a disease or disorder that inhibits proper RBC formation or production.
49. The method of paragraph 47 or 48, further comprising, prior to administering, the step of genetically modifying the induced RBC of b).
50. The method of paragraph 47 or 48, further comprising, prior to inducing, the step of genetically modifying the stem cell source of a).
51. The method of paragraph 49 or 50, wherein genetically modifying corrects a disease gene carried by the subject.
52. A kit comprising any of the compositions of paragraphs 1-16 and instructions for inducing a RBC using the composition.
53. A kit comprising the composition of paragraph 12, the composition of paragraph 13, and the composition of paragraph 14.
54. The kit of paragraphs 52 or 53, further comprising a stem cell source.
55. A kit for inducing a RBC comprising:
   a) a first composition comprising at least one of the components selected from the group consisting of: human AB plasma, an antibiotic, heparin, insulin, human holo-transferrin, a corticosteroid, and polyvinyl alcohol (PVA);
   b) a second composition comprising IL-3, SCF, and EPO;
   c) a third composition comprising SCF and EPO; and
   d) a fourth composition comprising EPO and optionally human holo-transferrin.

### EXAMPLES

### Example 1

### Introduction

Sickle Cell Disease (SCD) is a congenital blood disease caused by a single base-pair mutation in the adult β-globin gene (HBB^{Glu6Val}). Clinically, the disease manifests as severe anemia and bouts of painful vaso-occlusive crises, resulting in systemic tissue damage and increased mortality. Symptoms in patients do not appear until after birth, when a transcriptional phenomenon known as globin-switching occurs within bone marrow erythroblasts. Complex restructuring of the chromatin surrounding the β-globin locus on chromosome 11 results in the down-regulation of fetal (γ) globin gene expression, replaced by the adult (β) chain to form the adult hemoglobin tetramer (HbA). In patients with the sickle mutation, the mutant β-globin chain forms the sickle hemoglobin tetramer (HbS) that underlies the sickle cell anemia (SCA) disease phenotype. Under hypoxic conditions, HbS proteins found in circulating red blood cells (RBCs) polymerize into long chains that deform the biconcave RBC into the problematic “sickle” cell morphology.

Treatment options for SCD are currently limited to bone marrow transplantation, which is curative but carries significant risks, and the limited approved drugs available on the market, such as hydroxyurea. Otherwise, lifelong blood transfusions—risking infections, adverse reactions, and iron overload—are required for patients to sustain healthy blood hemoglobin levels. There is a need for safer and more targeted SCA therapies, but there is still a lack of adequate models available—in vivo or in vitro—that can fully recapitulate human globin-switching and the SCA disease phenotype, for which therapies could then be developed and trialed.

Methods of mimicking erythropoiesis in vitro have primarily relied on the use of immortalized cell lines with erythroid phenotypes, such as HUDEP-2 and BEL-A cells, or required erythroid differentiation of short-lived CD34⁺ progenitor cells from donated peripheral or cord blood. Obtaining CD34⁺ cells from SCD patients has historically been challenging due to the adverse effects of common mobilizing agents, such as G-SCF, which limits access to cellular materials needed for in vitro disease modeling and patient-specific therapeutic designs. While CD34⁺ cells from healthy donors can be manipulated to genetically model SCA, this practice ignores the larger genetic and epigenetic profiles in patients that contribute to the complex heterogeneity seen in disease symptoms, severity, and reactions to medications.

Human induced pluripotent stem cells (iPSCs) can be generated from easily-accessible, somatic cell sources, such as leukocytes (PBMCs) and skin (fibroblasts). iPSCs generated from SCD patients can provide a renewable cellular source from which CD34⁺ progenitor cells can be obtained using a number of hemogenic differentiation techniques, but subsequent differentiation of those progenitors into mature RBCs has so far been difficult due to a lack of terminal development. Engineered RBCs that present with an immature erythroid phenotype and fail to produce sufficient amounts of β-globin will not demonstrate the pathological traits of SCA needed for it to function as an in vitro disease model.

Described herein is an optimized culture protocol that efficiently engineers induced RBCs (iRBCs) from iPSCs, generated from healthy and SCD homozygous patient PBMCs. This protocol is used to generate an in vitro model of human SCA, composed of enucleated, HbS-positive, morphologically sickled iRBCs that mimic the disease phenotype. This protocol establishes a novel platform for the ex vivo production of patient-specific iRBCs from somatic cell-derived iPSCs to serve as models of red cell diseases, on which new therapies can be developed and trialed.

### Materials and Methods

iPSC generation and maintenance—Human induced pluripotent stem cells (iPSCs) were generated from two unrelated healthy (WT) and two unrelated sickle homozygous (SCA) patient peripheral blood mononuclear cells (PBMCs), as previously described; see e.g., Doulatov et al. Sci Transl Med. 2017; 9(376): eaah5645; the content of which is incorporated herein by reference in its entirety. iPSC lines were karyotyped and genotyped using Sanger sequencing to confirm the presence or absence of a homozygous β-globin sickle cell mutation (HBB^{Glu6Val}) (see e.g.,FIG. 5A). iPSC colonies were maintained in mTeSR (STEMCELL Technologies, USA) on MATRIGEL-coated culture plates in standard normoxic incubating conditions (20% O₂, 5% CO₂, 37° C.) with daily media changes. No feeder layers were used in iPS growth or any differentiation step hereafter.

Hemogenic differentiation of iPSCs into CD34⁺ progenitor cells—Healthy (WT) and SCD patient-derived (SCA) iPSCs underwent 2D hemogenic differentiation on MATRIGEL-coated plates using the STEMDIFF Hematopoietic kit (STEMCELL Technologies, USA), as per manufacturer's instructions. On Day 12 of the STEMDIFF protocol, floating progenitor cells with hematopoietic potential were isolated using the human CD34 MicroBead kit (MACS, MILTENYI BIOTEC, Germany), as per manufacturer's instructions. Manual cell counts following CD34 enrichment confirmed both iPSC genotypes generated similar numbers of CD34⁺ progenitor cells per plate (see e.g., FIG. 5B) with similar CD34/CD45/CD36 immunophenotypic profiles, when analyzed by flow cytometry (see e.g., FIG. 5C, FIG. 5D).

Erythroid differentiation using the EDM formula—erythroid differentiation from iPSC-derived CD34⁺ progenitor cells took place in three erythroid differentiation stages (ED I, ED II, and ED III) using an erythroid differentiation media (EDM); see e.g., Huang et al Nat Commun. 2017; 8(1):423, 2017; the content of which is incorporated herein by reference in its entirety. In the BSA EDM, all ED stages used a basal media that consisted of IMDM (ISCOVE'S DMEM, CORNING, USA), 2% Pen/Strep (CORNING, USA), 1% BSA (Fraction V 7.5%, GIBCO, USA), 5% human plasma, heat-shocked (STEMCELL Technologies, USA) or 5% human AB serum (VALLEY BIOMEDICAL INC, USA), 3U human heparin (ACROS DIAGNOSTICS, Belgium), 10 μg/mL insulin (SIGMA-ALDRICH, USA), 250 μg human holo-transferrin (SIGMA-ALDRICH, USA), and 500 nM dexamethasone (SIGMA-ALDRICH, USA). Stage-specific supplements added to the basal media at each ED stage were as follows: ED I (Day 0-7): 2 ng/mL IL-3 (R&D SYSTEMS, USA), 10 ng/mL SCF (R&D SYSTEMS, USA), 3 U/mL EPO (Life Technologies, USA); ED II (Day 7-10): 10 ng/mL SCF, 1 U/mL EPO; ED III (Day 10-18): 0.2 U/mL EPO, 300 μg/mL human holo-transferrin. The PVA EDM formula contained all the same media components and supplements as a 5% plasma EDM, except for the substitution of BSA with 0.8% w/v polyvinyl alcohol (87%-89%; SIGMA-ALDRICH, USA). Erythroid differentiation took place in ultra-low adherence plates and cells were kept in high density throughout (0.5×10⁵/mL to 2.5×10⁵/mL in ED I; 1×10⁵/mL to 2.5×10⁵/mL in ED II; 1×10⁶/mL to 5×10⁶/mL in ED III). Media changes occurred every 2-3 days with fresh, pre-warmed media. Incubation occurred in standard normoxic conditions (20% O₂, 5% CO₂, 37° C.), except during hypoxic conditioning, when plates were kept in a low-oxygen chamber (5% O₂, 5% CO₂, 37° C.).

Real-time quantitative PCR (RTqPCR)—Semi-quantitative mRNA expression of human globins was performed on iRBC cDNA using the GoTaq® SYBR-Green Master Mix kit (PROMEGA, USA) and processed by the QUANTSTUDIO 7 FLEX (APPLIED BIOSYSTEMS, USA). The comparative CT method was used to determine relative mRNA expression levels. Primers used for RTqPCR are listed (see e.g., Table 1).

TABLE 1
RTqPCR primers

| Name | Sequence (5’-3’) | SEQ ID NO: |
|---|---|---|
| Human β-Actin FOR | GTG CTG TCC CTG TAT GCC TCT G | 1 |
| Human β-Actin REV | GGG AGA GCA TAG CCC TCG TAG ATG | 2 |
| Human β-globin FOR | CGT GCT GGT CTG TGT GCT G | 3 |
| Human β-globin REV | CCC CCA GTT TAG TAG TTG GAC TTA GGG | 4 |
| Human γ-globin FOR | GGA CCC AGA GGT TCT TTG ACA GC | 5 |
| Human γ-globin REV | GGA AGT CAG CAC CTT CTT GCC | 6 |
| Human ε-globin FOR | GCC AGA ACT TCG GCA GTA AAG AAT | 7 |
| Human ε-globin REV | CCT GAG AGC TTG CTA GTG ATT G | 8 |
| Human α-globin FOR | TCC TAA GCC ACT GCC TGC TG | 9 |
| Human α-globin REV | TTA ACG GTA TTT GGA GGT CAG CAC | 10 |

High-performance liquid chromatography—1×10⁶ to 5×10⁶ iRBCs were harvested from cultures on Day 14, 16 and 18 of EDM and washed three times in PBS to remove all media. Cell pellets were then lysed in dH₂O with three freeze-thaw cycles. Lysed samples were centrifuged and the supernatant was extracted and stored at −80° C. until testing. Samples were diluted 1:5 in D-10 diluent solution (BIO-RAD, USA) and run on the D-10 Hemoglobin Testing System (BIO-RAD, USA) set to the HbA2/F program.

RNA Sequencing and Analysis—1×10⁵ peripheral blood CD34⁺ derived cultured red cells (cRBC), as well as WT and SCA iPSC-derived iRBCs were analyzed at Day 11 and Day 15 of erythroid differentiation in triplicate to form RNAseq libraries. Sequencing was carried out on an ILLUMINA NEXTSEQ 500 on a single read flowcell with 75 cycles. Analysis was performed using a Cancer Computational Biology (CCCB) custom pipeline. Raw sequencing reads were aligned to the hg19 reference genome using an RNA-specific Spliced Transcripts Alignment to a Reference (STAR) aligner. Quality control was done with FastQC. Read counts were calculated using the featureCounts in the Subread package. Differential expression analysis was performed using the DESeq2 package in R and subsequent analyses were conducted using Gene Ontology (GO) and Gene Set Enrichment Analysis (GSEA) in their native implementations. Comparisons to publicly available data from GEO (GSE53983) used r-log normalization from DEseq2 and dimensionality reduction via PCA using the pr-comp function in the R stats package. Data is publically available from GEO (GSE125196).

Phalloidin assay—1×10⁵ iRBCs (WT and SCA) or adult peripheral blood red cells (AA and SS) were stained with a FITC-conjugated phalloidin peptide (LIFE TECHNOLOGIES, USA) in an isotonic (300 mOsm/L) solution for 10 minutes at 37° C. Flow cytometry was then used to quantitate the proportion of phalloidin-positive cells within the live (propidium iodide-negative), enucleated (DNA-negative) red cell populations.

### Results

Human plasma is crucial for the growth and development of iRBCs from human iPSCs.

In the goal to generate terminally mature red blood cells from human iPSCs, and to subsequently model SCA in vitro, key components of a modified erythroid differentiation media (EDM) were trialed and tested for optimization. In order to determine whether human serum or human plasma served as the superior media supplement, a BSA-based EDM containing either 5% human serum or 5% human plasma was used to test which supplement generated viable, mature erythrocytes based on phenotype, globin content, and morphological changes. iPSC-derived CD34⁺ progenitors from healthy (WT) and sickle homozygous (SCA) patients underwent a three-stage in vitro erythroid differentiation (ED) protocol using the serum- or plasma-enriched EDM (see e.g., FIG. 1A).

For both WT and SCA cells, plasma was found to be a key driver of cell growth and a necessity for maintaining cell viability throughout all ED stages (see e.g., FIG. 1B). Proliferation of erythroblasts of both genotypes was significantly greater in a plasma-supplemented EDM compared to serum, typically peaking in numbers at Day 14 and stagnating once enucleation commenced. Serum alone was a poor promoter of erythroid cell growth in vitro, and high levels of cell death were visible during manual counts from Day 10 onwards. By Day 18 of the EDM protocol, few viable cells were found in serum-supplemented cultures, indicating human plasma contained pro-viability and pro-erythropoietic factors that serum lacked.

Erythroid lineage specificity was also accelerated in the presence of plasma (see e.g., FIG. 1C-1E). The emergence of common erythroid markers, GlyA (CD235a), Band 3, and CD71, identified by flow cytometry, were used to track erythropoietic development over time. Cells of both genotypes rapidly acquired these markers when grown in the plasma EDM, becoming >95% GlyA⁺ and >90% Band 3⁺by Day 18. In serum, expression of these markers was significantly lower, and decreased towards the terminal stages of development when cell viability also began to decline. Interestingly, the rise and fall of CD71 expression was uniquely genotype-specific; SCA iRBCs retained more CD71 than their WT counterparts as they matured, regardless of media conditions (see e.g., FIG. 1E). Since CD71 is integral for hemoglobin synthesis, this observation indicated SCA cells were showing signs of anemia towards their terminal stages of development.

Enucleation was an important indicator of iRBC maturity and was typically observed from Day 14 onwards using the EDM protocol. Enucleation was quantitated by flow cytometry using GlyA and a membrane-penetrant DNA dye (Hoecsht) (see e.g., FIG. 6A). In a plasma-supplemented EDM, both WT and SCA iRBCs successfully enucleated at a high rate (65%-85%), while in serum, most cells failed to pass this maturation milestone (see e.g., FIG. 1F). Of the enucleated iRBCs, the proportion of true red blood cells to reticulocytes, based on Thiazole Orange detection of cytoplasmic RNA (see e.g., FIG. 6B), demonstrated that reticulocytes dominated the final culture population, regardless of media type or genotype (see e.g., FIG. 1G).

Microscopy was performed at each ED stage to track maturation and morphological changes of the iRBCs (see e.g., FIG. 1H). Smears of plasma-supplemented cultures showed rapid erythropoietic development and patterns of morphological evolution that resembled in vivo human erythropoiesis, with enucleated erythrocytes prominently appearing in the culture from Day 14 onwards. In serum-supplemented cultures, few enucleating cells were seen and a notable presence of underdeveloped erythroblasts were still present at later time-points. Both light microscopy and electron microscopy were able to capture individual enucleation events, providing detailed imagery of pyrenocyte expulsion, forming the reticulocyte (see e.g., FIG. 14

Together, this demonstrated that iPSC-derived progenitor cells can undergo mature erythropoietic processes in vitro to produce red blood cells that phenotypically and morphologically resemble native RBCs found in vivo, and that plasma plays a vital role in those processes.

iRBCs undergo globin-switching and model human Sickle Cell Anemia in vitro.

Replicating the globin-switching phenomenon that occurs in vivo was paramount to achieving a human model of globinopathy in vitro. Globin content was tracked in plasma-EDM iRBC cultures throughout the ED protocol at both an RNA and protein level. Here, the goal was to identify how normoxic (20% O₂) and hypoxic (5% O₂) incubation environments impacted globin expression patterns, with the aim to achieve the most robust expression of adult β-globin.

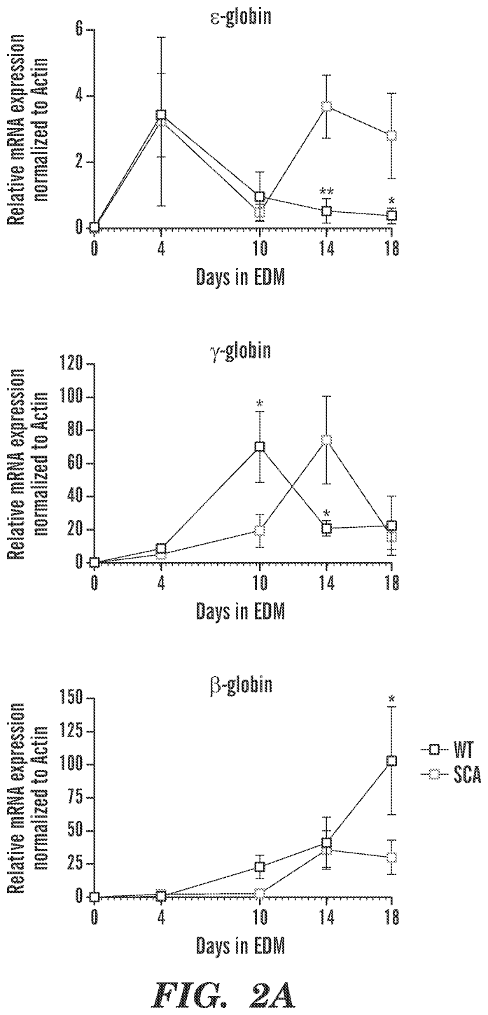

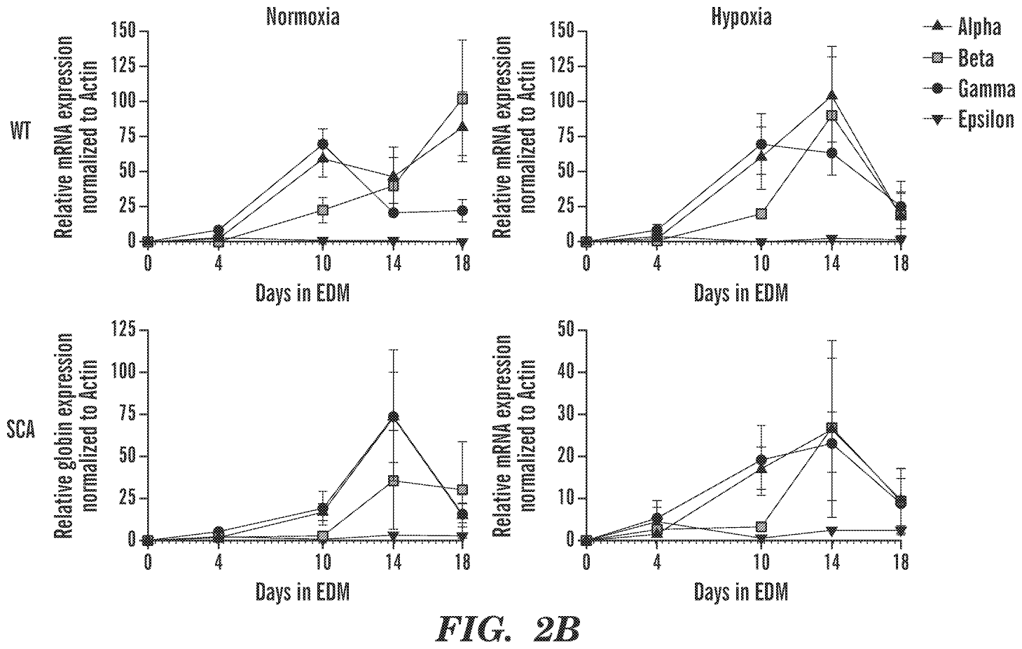

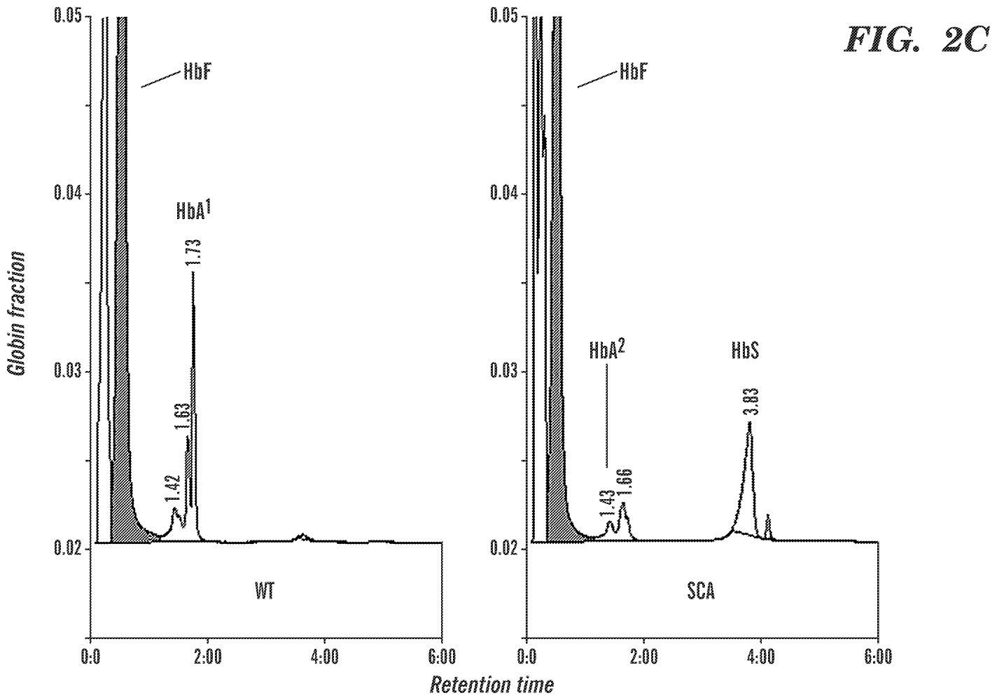

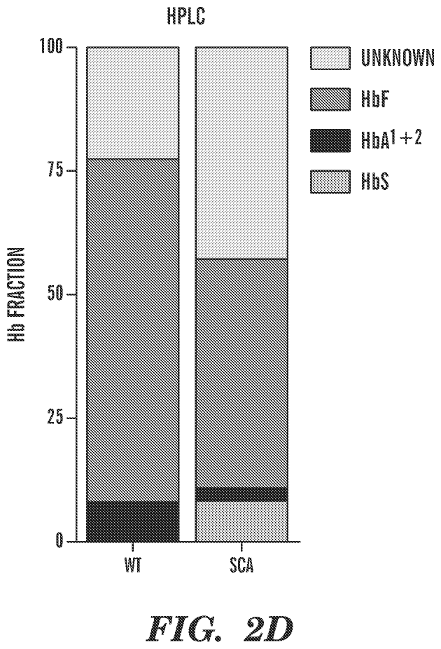

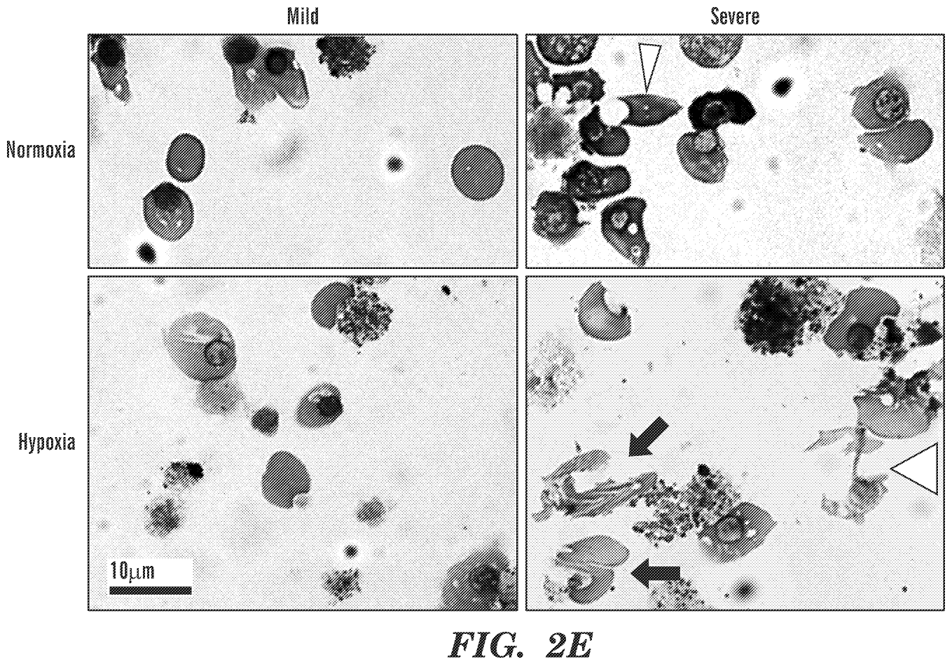

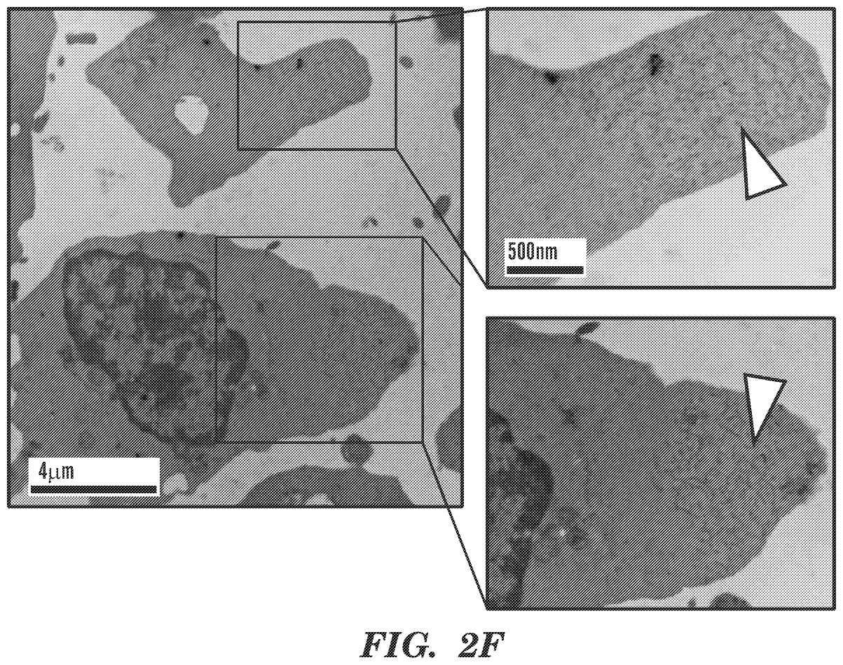

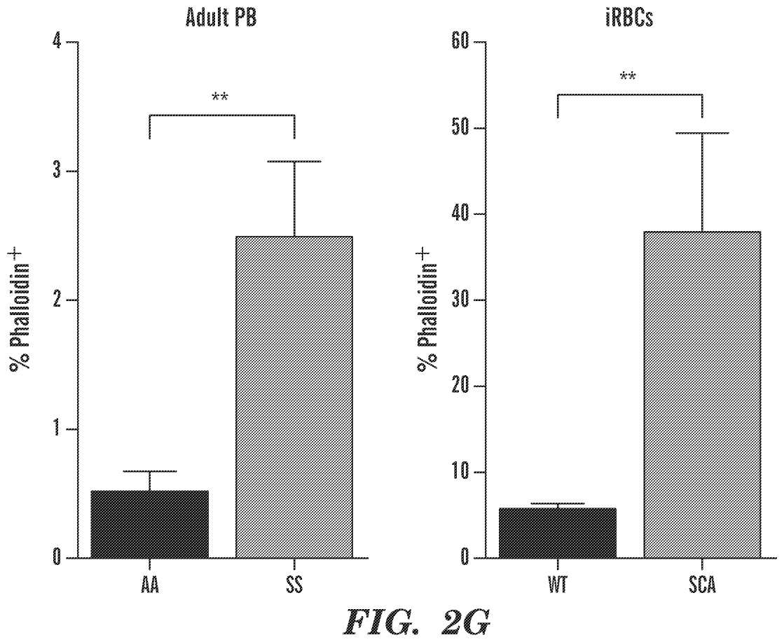

Using real-time quantitative PCR (RTqPCR), relative mRNA expression levels of the key human beta-like globin chains—ϵ (epsilon), γ (gamma), and β (beta)—were measured in WT and SCA cells over time (see e.g., FIG. 2A). At an mRNA level, WT cells underwent three distinct waves of globin chain activation and deactivation that closely mimicked the human in vivo globin-switching map (see e.g., FIG. 2A, FIG. 2B); see e.g., Iarovaia et al. Biochemistry (Mosc). 2018, 83(4):381-392, the content of which is incorporated herein by reference in its entirety. A notable transcriptional switch from fetal (y) to adult 03) globin was seen in WT iRBCs just prior to Day 14, when enucleation also commenced. By Day 18 of the erythroid differentiation protocol, WT iRBCs were roughly 80% β-globin-expressing at an mRNA level.

SCA iRBCs also developed into β-globin-expressing erythrocytes; however, their pattern of globin-switching was notably different to WT counterparts (see e.g., FIG. 2A, FIG. 2B). At an mRNA level, there was a delay in the activation of adult β-globin expression, with a much longer persistence of γ-globin transcription and a small reappearance of ϵ-globin at later time-points. This pattern indicated a compensatory mechanism was initiated within maturing SCA iRBCs when the effects of the β-globin sickle mutation became apparent. While adult globin chain expression was notably weaker than in WT iRBCs, this showed that both genotypes had the capacity to produce β-globin positive iRBCs.

Hypoxic incubation, which was trialed between Day 10 and Day 14 of EDM, had a favorable effect on globin-switching in WT cells (see e.g., FIG. 2B). Compared to normoxic counterparts, hypoxia induced a two-fold to three-fold increase in relative α-globin mRNA and β-globin mRNA expression in WT iRBCs prior to enucleation. SCA cells did not perform as well in hypoxia in terms of globin synthesis (see e.g., FIG. 2B). Compared to normoxic counterparts, relative β-globin mRNA expression in SCA iRBCs was reduced by half. Given the sickle mutation's known effect on red cell oxygen-carrying capacity, it was not unexpected that SCA iRBCs were poorly equipped to handle prolonged hypoxic stress.

High performance liquid chromatography was performed on post-enucleation iRBCs in order to identify the presence of hemoglobin protein tetramers, such as HbF, HbA, and HbS (see e.g., FIG. 2C). In WT iRBCs, HbF and HbA (including both HbAl and HbA2) were the key eluted hemoglobins, agreeing with mRNA data that mature iRBCs produced adult globin chains such as β- and δ-globin. In the SCA cells, the unique HbS mutant protein was clearly identified using HPLC, along with HbA2, while HbAl was not detectable due to the homozygous sickle mutation (see e.g., FIG. 2C). The averages of hemoglobin fractions derived from HPLC data is shown (see e.g., FIG. 2D). While HbF was the dominant protein found in both genotypes, HbA made up an average of 7.7% of the hemoglobin profile of WT iRBCs, while HbS made up 8.1% of the total hemoglobin of SCA iRBCs. Unknown peaks and fractions were likely from cellular debris. HPLC data demonstrated that iRBCs engineered from patient iPSCs have the capacity to generate adult hemoglobin proteins, including HbA and HbS, that are detectable using the gold standard method used to diagnose globinopathies in patients; see e.g., Sankaran et al. Science. 2008, 322(5909):1839-1842, the content of which is incorporated herein by reference in its entirety.

A model of human Sickle Cell Anemia is also reliant on mimicking the morphological characteristics of the diseased “sickle” red cell, which appears in vivo under hypoxic conditions due to the polymerizing action of HbS proteins; see e.g., Papageorgiou et al. Proc Natl Acad Sci U S A. 2018;115(38):9473-9478, the content of which is incorporated herein by reference in its entirety. Microscopy was performed on SCA iRBCs following incubation in either normoxia or hypoxia incubators for a period of 48 hours (see e.g., FIG. 2E). Under both oxygen conditions, a combination of iRBCs bearing either a “mild” or “severe” sickle phenotype was observed. Amongst sickling, other pathological observations included microcytosis, hypochromasia, fragmentation, and cell-to-cell adhesion, which were more severe and more frequently seen in hypoxic SCA iRBCs. High-magnification electron micrographs (EMs) of hypoxic sickled iRBCs showed long, protein polymers formed within the elongated cytoplasm of deformed cells, supporting the findings under light microscopy that the sickle morphologies were a result of the mutant HbS proteins polymerizing within SCA iRBCs (see e.g., FIG. 2F). These polymers were not visible in EMs of WT iRBCs (see e.g., FIG. 1I).

SCA iRBCs were further characterized functionally for their membrane integrity using a phalloidin permeability assay. Phalloidin only permeates weak or damaged cellular membranes and binds to intracellular F-actin with high affinity. As shown in peripheral blood samples taken from healthy (AA) and sickle cell homozygous (SS) patients, the higher deformability of sickle cells allowed greater phalloidin binding to red cell actin filaments (see e.g., FIG. 2G). This pattern was mimicked in iPSC-generated red cells, as SCA iRBCs showed a significant increase in the percentage of phalloidin-positive cells compared to WT cells due to the higher deformability, and therefore permeability, of SCA iRBC cellular membranes (see e.g., FIG. 2G). This live assay complemented visual observations that SCA iRBCs undergo sickling in vitro, and therefore are a functional model of human Sickle Cell Anemia.

RNA-sequencing identifies perturbed oxygen and metabolic pathways in SCA iRBCs.

In order to test the applicability of iRBCs in human disease studies, iRBCs derived from WT or SCA patients were compared on a transcriptional level in order to identify additional disease phenotypes that may be present. Bulk RNA-seq was performed on early (Day 11) and late (Day 15) stage iRBCs from both genotypes, developed in plasma-supplemented EDM under normoxic conditions. Control RBCs (cRBCs), differentiated from peripheral blood (PB) CD34⁺ progenitors, using the same protocol, served as controls to assess how similar the iPSC-derived iRBCs were at an mRNA level to cRBCs derived from other sources. All iRBCs and cRBCs used for these analyses were positive for RNA (i.e., reticulocytes).

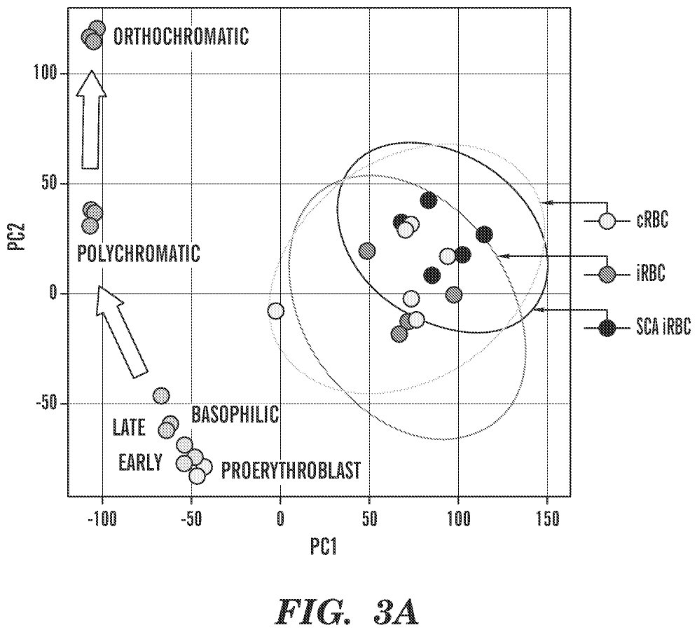

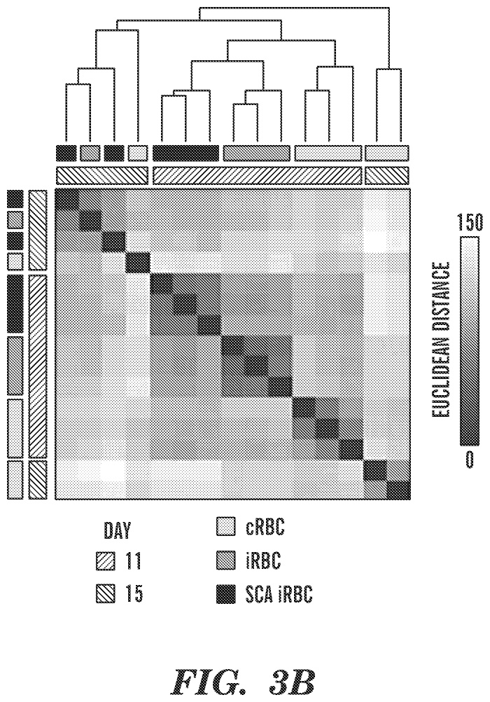

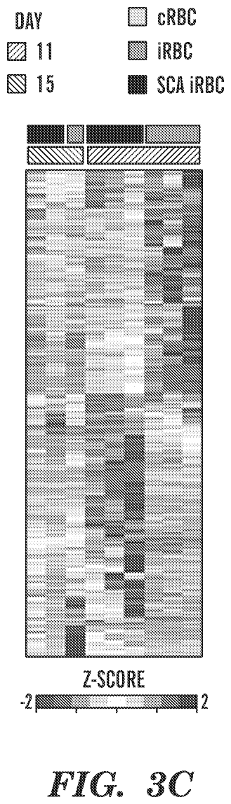

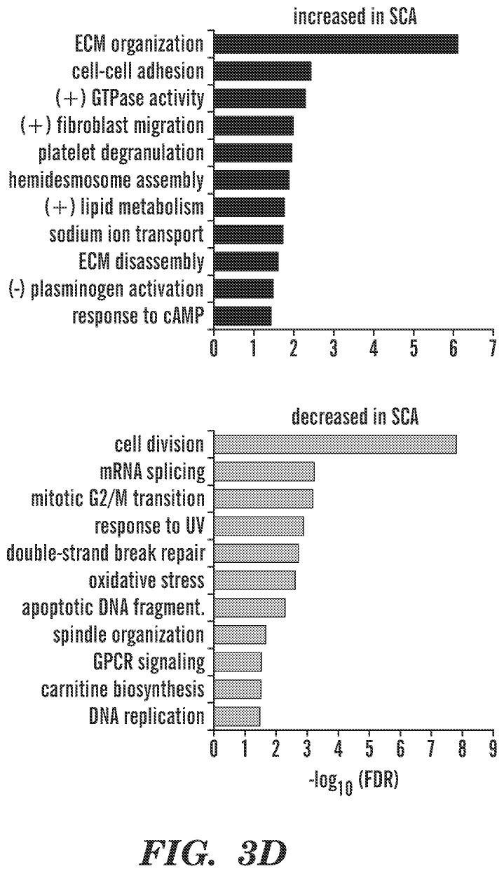

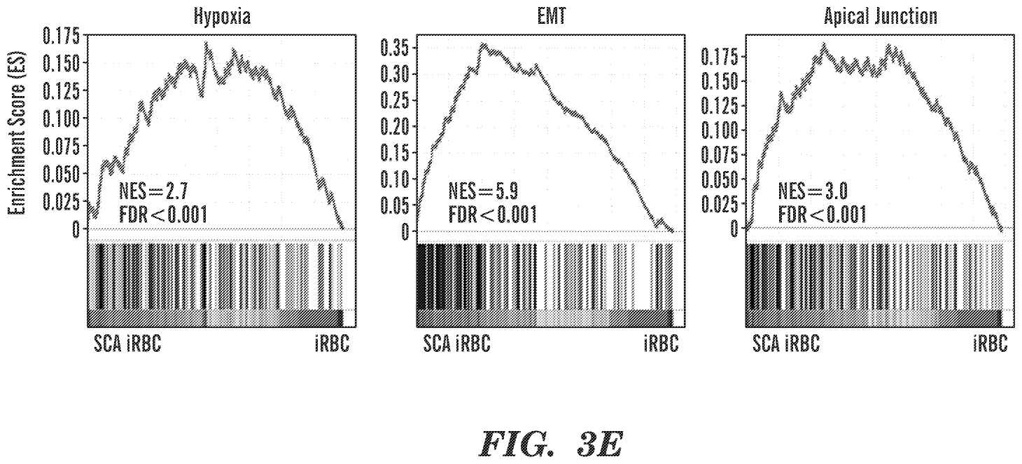

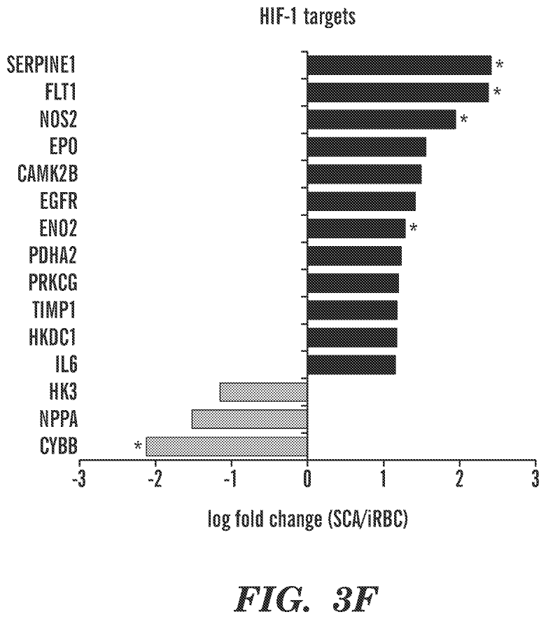

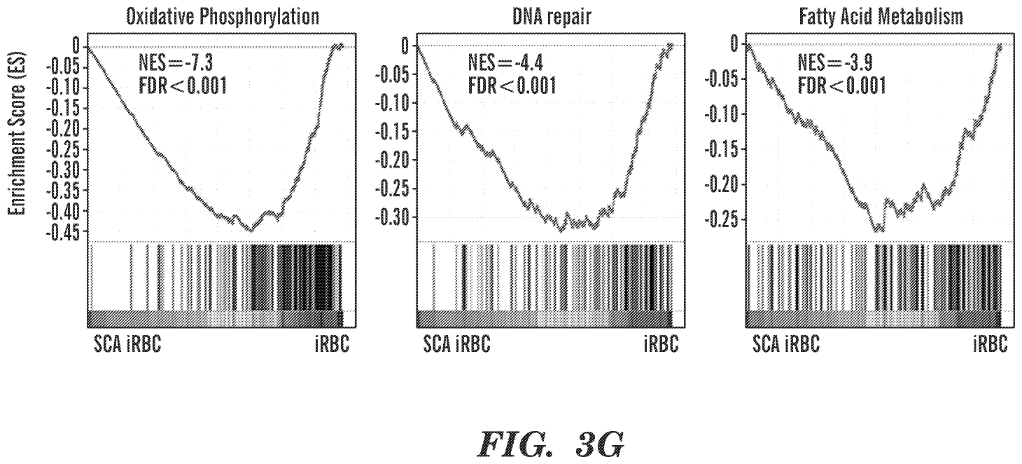

Dimensionality reduction techniques, captured at Day 11 and Day 15 of EDM on the second principal component axis, indicated that iRBCs and cRBCs exhibited near-identical transcriptome profiles (see e.g., FIG. 3A). This was true for both iRBC genotypes, which each exhibited 95% overlapping cell type-specific confidence ellipses to the PB-derived cRBCs. A more resolved clustering of the Euclidean distances between individual samples indicated that the variance in mRNA transcription between the two stages of erythroid differentiation (Day 11 versus Day 15) was more pronounced, indicating a transcriptional shift occurring throughout erythroid development that coincided with key erythroid phenotypic changes that distinctly separated these time-points (see e.g., FIG. 3B). Variance between WT and SCA iRBCs, and also cRBCs, was less apparent at Day 15, demonstrating that iPSC-derived iRBCs and PB-derived cRBCs were near-identical at a transcriptional level towards the terminal stages of their development.

When comparing the transcriptional profiles of WT versus SCA iRBCs, RNA-seq detected 751 differentially expressed genes between the two groups (see e.g., FIG. 3C). Gene ontology (GO) analysis indicated SCA iRBCs had high expression levels of genes related to extracellular matrix (ECM) organization and cell-to-cell adhesion (see e.g., FIG. 3D), which aligned with earlier observations regarding SCA iRBC sickling in vitro. Down-regulated genes in the SCA iRBCs were associated with cell proliferation and DNA repair pathways (see e.g., FIG. 3D). Additionally, hypoxia response genes, many of which are HIF-la targets, were significantly elevated in SCA iRBCs (see e.g., FIG. 3E, FIG. 3F), despite normoxic conditions used for these tests. This was consistent with earlier findings that showed SCA iRBCs have increased sensitivity to hypoxic stress compared to WT cells.

Other metabolic pathways found to be aberrant in SCA iRBCs were less known to the classical disease phenotype, such as lipid metabolism (see e.g., FIG. 3G). Though poorly understood mechanistically, SCA patients have been found to exhibit hypocholesterolemia and hypertriglyceridemia; see e.g., Zorca et al. Br J Haematol. 2010, 149(3):436-445; Shores et al. J Natl Med Assoc. 2003, 95(9):813-817; Buchowski et al. JPEN J Parenter Enteral Nutr. 2007, 31(4):263-268; the contents of each of which are incorporated herein by reference in their entireties. These additional phenotypes, captured through RNA-seq, broaden understanding of the wide spectrum of pathologies seen in SCA patients, and may serve to be future therapeutic targets. This further demonstrates how an in vitro model of SCA, derived from somatic cell sources, is a valuable tool for patient-specific genetic and clinical research.

A xeno-free EDM containing PVA further enhances iRBC proliferation.

Polyvinyl alcohol (PVA) has recently undergone investigations as a viable replacement for bovine serum albumin (BSA) in culture medias, and has shown to have a positive effect on human stem cell proliferation in vitro; see e.g., Wilkinson et al. Nature. 2019, 571(7763):117-121 (published correction appears in Nature. 2019 Jul;571(7766): E12), the content of which is incorporated herein by reference in its entirety. In an attempt to further optimize the erythroid differentiation protocol and eliminate xenoid products from the EDM formula, it was tested how well PVA served as a substitute for BSA in the plasma-based EDM. A concentration of 0.08% w/v PVA was the optimal replacement for 1% v/v BSA in this system. All other components of the EDM and the three-stage erythroid differentiation protocol remained identical (see e.g., Methods, supra).

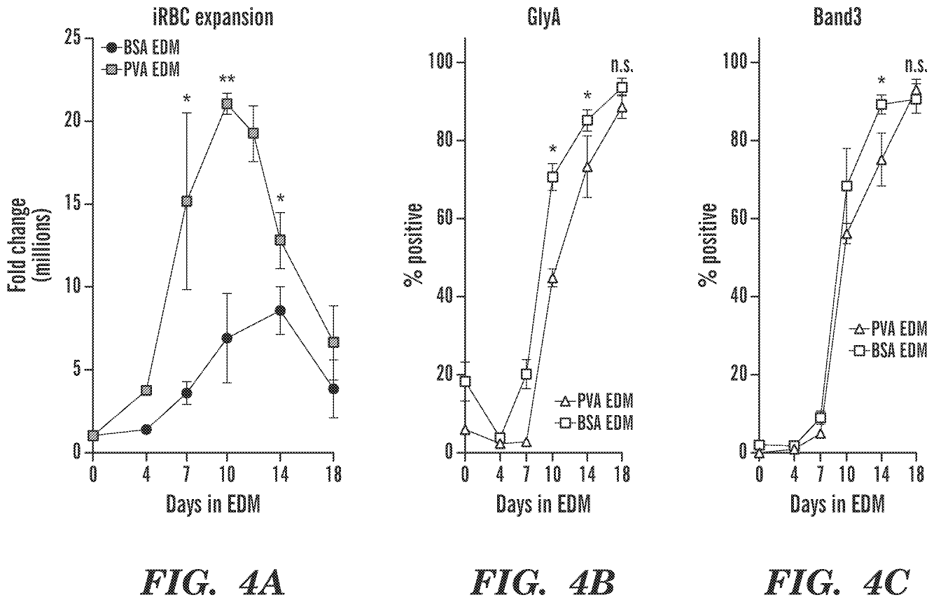

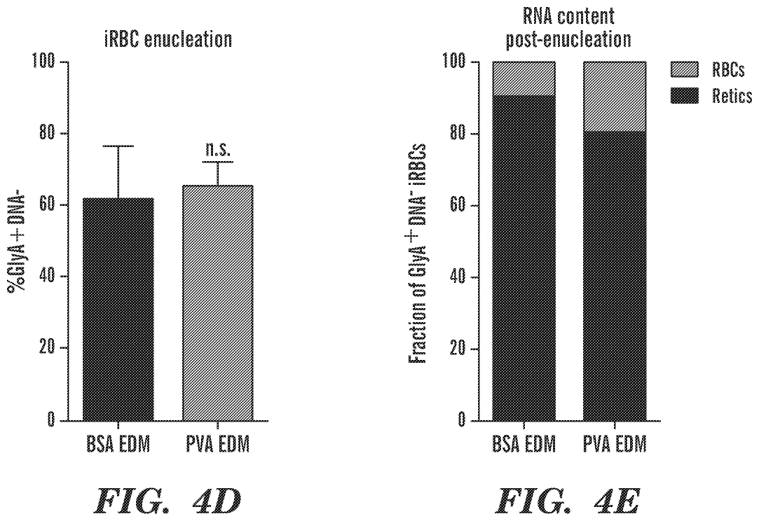

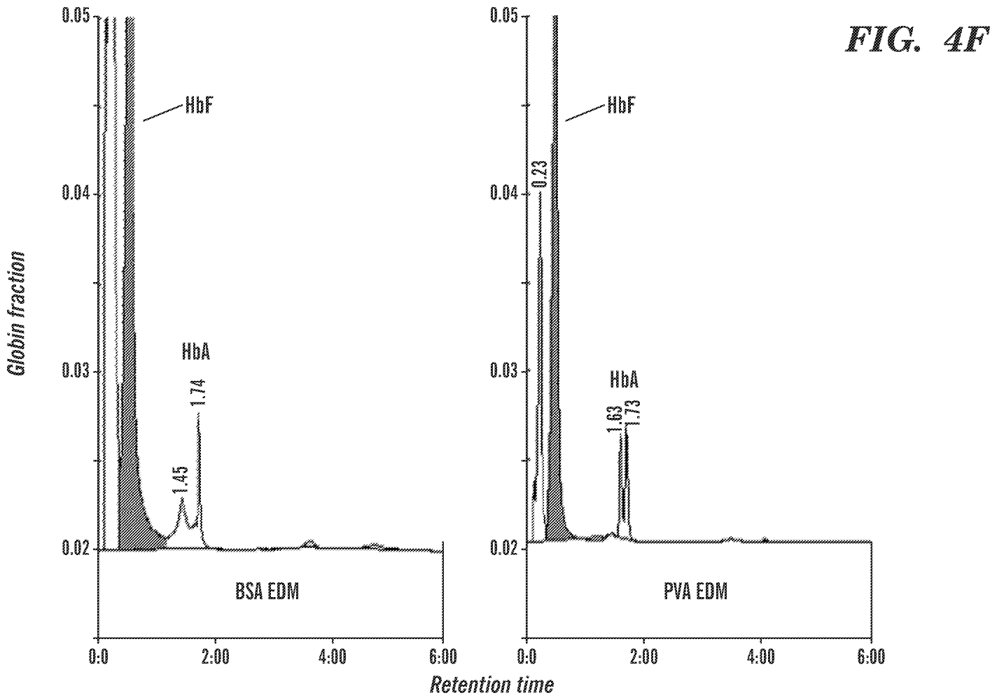

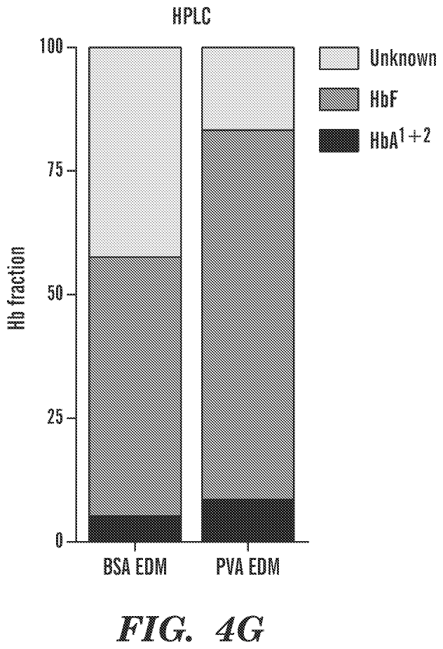

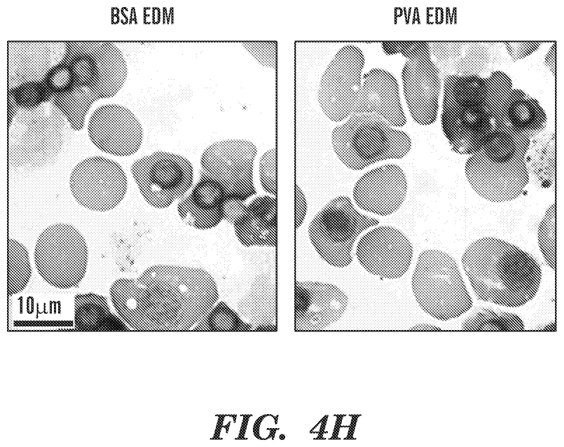

A xeno-free EDM, containing PVA, had a significant effect on early erythroid proliferation, resulting in a three-fold to five-fold increase in cell numbers compared to those cultured in BSA EDM (see e.g., FIG. 4A). In PVA, the acquisition of erythroid markers, GlyA and Band 3, was noticeably slower in the early stages of development compared to BSA, indicating erythroid cells were kept at a highly proliferative, early erythroblast state longer before maturing (see e.g., FIG. 4B, FIG. 4C). However, terminal maturation of iRBCs was not hindered by PVA, and by Day 18 of the protocol, both EDMs produced iRBCs that were similarly 90-95% GlyA⁺Band3⁺ in phenotype.

Terminal erythropoietic processes remained equally as successful in a PVA-based EDM compared to the BSA EDM, such as identical rates of enucleation (see e.g., FIG. 4D). The ratio of RBCs to reticulocytes within the enucleated population was more favourable in PVA EDM compared to BSA EDM (see e.g., FIG. 4E); however, both cultures remained dominated by reticulocytes by the final day of development, as previously observed.

Globin protein analysis performed by HPLC showed both EDM formulas generated iRBCs containing adult hemoglobin tetramers, with the average fractions of HbA being 6.5% in BSA and 8.3% in PVA (see e.g., FIG. 4F). Smears of both cohorts appeared identical under light microscopy, as both EDM formulas generated round, enucleated iRBCs of similar size and unremarkable morphology (see e.g., FIG. 4G). This demonstrated that a PVA-based EDM had the same capacity to differentiate iPSC-derived progenitors into mature, enucleated, hemoglobinized iRBCs in vitro as a traditional BSA-based EDM. The enhanced proliferative properties of PVA on erythroid progenitors also make this xeno-free substitution an attractive choice for investigators who are interested in bulk iRBC production for translational uses.

CRISPR-Corrected iRBCs resemble wild-type iRBCs

iPS cells samples were obtained from SCA patients. The CRISPR-Cas9 gene editing system was employed to “correct” the cells, i.e., CRISPR was used to remove the homozygous sickle mutation (HBBE6V) from the patient iPS cells, and the corrected, wild-type genetic sequence was added in its place. These CRISPR-corrected iPS cells were used to induce RBCs using the methods described herein. Data presented herein show that CRISPR-corrected cells were capable of forming “healthy” RBCs, essentially identical to iRBCs generated from the iPSCs of healthy patients (see e.g., FIG. 7). If it further noted that HbS is markedly reduced and HbA is increased in CRISPR-corrected cells as compared to SCA, confirming that the correction has occurred and the disease has been eliminated (see e.g., FIG. 9C).

Growth was identified as being a positive feedback loop, meaning the greater number of cells as starting material, the faster they grow. As a non-limiting example, starting with at least 2.5×10⁵/mL (e.g., as compared to starting with at least 0.5×10⁵/mL, as described herein) iPSC-derived CD34⁺ progenitor cells in the ED I medium can yield increased numbers of resultant iRBCs using the methods described herein. As another non-limiting example, using at least 2.5×10⁵/mL (e.g., as compared to using at least 1.0×10⁵/mL, as described herein) ED I-derived cells in the ED II medium can yield increased numbers of resultant iRBCs using the methods described herein. As another non-limiting example, using at least 5.0×10⁶/mL (e.g., as compared to using at least 1.0×10⁶/mL, as described herein) ED II-derived cells in the ED III medium can yield increased numbers of resultant iRBCs using the methods described herein. Discussion

The aim of this project was twofold: first, to optimize a culture protocol that had the capacity to use human iPSCs, derived from somatic cell sources, to engineer fully mature, hemoglobinized iRBCs in vitro. Second, to use that protocol to generate a new model of human Sickle Cell Anemia (SCA) with comparable phenotypic properties to the human disease, suitable for basic and translational studies.

Differentiation protocols for generating RBCs ex vivo vary greatly; see e.g., Vinjamur et al. Methods Mol Biol. 2018, 1698:275-284; Uchida et al. Mol Ther Methods Clin Dev. 2018, 9:247-256; Giarratana et al. Nat Biotechnol. 2005, 23(1):69-74; Baek et al. Transfusion. 2008, 48(10):2235-2245; Huang et al. Nat Commun. 2017, 8(1):423; the contents of each of which are incorporated herein by reference in their entireties. Variations of an erythroid differentiation media (EDM) were tested, and it was determined that components such as human plasma contained vital pro-erythropoietic and pro-viability factors that serum alone lacked, making plasma an absolute necessity in an erythroid monoculture medium. An optimized EDM and a three-stage erythroid differentiation (ED) protocol allowed for the terminal maturation of iPSC-derived progenitors into GlyA⁺ Band3⁺CD71^{lo} iRBCs, which enucleated at a high rate (e.g., up to 85%) and underwent globin-switching to produce adult hemoglobins. iPSCs harboring the HBBG^{Glu6Val} mutation in homozygosity produced morphologically sickled iRBCs that displayed pathological profiles similar to those seen in SCD patients, which were exacerbated by environmental hypoxic stress. HPLC, a gold standard diagnostic tool for globinopathies, was capable of detecting HbA and HbS proteins within WT and SCA iRBCs respectively. Lastly, the plasma-based EDM was further optimized into a xeno-free version by replacing BSA with polyvinyl alcohol (PVA), which significantly increased early erythroblast proliferation and generated clinical-grade iRBCs. These engineered red cells can be utilized to study normal erythropoietic processes in vitro that still require investigation, such as globin-switching and pyrenocyte ejection, as well as serve as models for any number of congenital human red cell diseases.

The generation of an in vitro model of Sickle Cell Anemia that morphologically and transcriptionally recapitulates the human condition has profound implications for novel drug design and pre-clinical testing. Using somatic cells from patients (in this case, leukocytes) as the iPSC source simplified the process of creating patient-specific models without the need for mobilizing agents to gain hematopoietic progenitors, and allowed for continuous iRBC production in culture. Multiple studies of the use of the FDA approved drug, hydroxyurea, have highlighted how diverse the SCD population is in terms of phenotypes, affecting patient dosage, efficacy, and safety; see e.g., Lanzkron et al. Ann Intern Med. 2008,148(12):939-955; Sclafani et al. Hematol Rep. 2016, 8(4):6678; Pule et al. Clin Transl Med. 2016, 5(1):15; the contents of each of which are incorporated herein by reference in their entireties. RNA-sequencing further demonstrated that individual patients can present with rare or uncharacterized phenotypes that may impact responses to new medications, or act as predictors for new therapeutic targets. This model is particularly beneficial for patients who are non-responders or experience adverse reactions to current medications, and for the testing of new medications in development, such as Voxelator; see e.g., Estepp et al. Am J Hematol. 2018, 93(3):326-329; Vichinsky et al. N Engl J Med. 2019, 381(6):509-519; the contents of each of which are incorporated herein by reference in their entireties. Phalloidin permeability, as an assay to assess cell sickling, can serve as a rapid read-out for testing. Ongoing investigations using hydroxyurea and Voxelator on iRBCs can be performed, as well as the generation of a CRISPR-corrected iPSC line that rescues key phenotypes of the SCA model. Healthy iRBC production also has clinical use as a substitute for donor blood products, such as those used in transfusions to treat disease or injury.

### Example 2

### Transfusion of Human iRBCs in Anemic Mice (In Vivo Trial)

Experimental setup: 5× NSG (immune-compromised) mice, all 6-8 weeks old, were administered 1× dose of full-body Cesium irradiation at 300cGy (non-lethal). There was 48 hours of recovery from irradiation before any treatment. Each mouse was given one of five different treatment plans (randomized, single-blinded): (1) No irradiation, no treatment; (2) Irradiation, but no treatment; (3) Irradiation, IV injected with 100 uL Saline; (4) Irradiation, IV injected with 100 uL mouse blood (from a non-irradiated healthy donor mouse of same age); or (5) Irradiation, IV injected with 100 uL human WT iRBCs. iRBCs were generated by the XF-EDM protocol from human iPSCs, as described herein. iRBCs were pre-analyzed and sorted before injection (i.e., only enucleated (DNA-negative) red cells were transfused). Roughly 45-65 million red cells total (either mouse or human) were injected into mice. The blood stats and health of the mice were tracked routinely over a period of 14 days (see e.g. FIG. 12). All mice were humanely euthanized at the end of the experiment.

Experimental timeline: On Day −7, baseline blood values were obtained. On Day 0, irradiation (300cGy) was performed. On Day 3, mice given treatment (e.g., nothing, saline, or blood transfusion). On Day 7, mice were euthanized.

Conclusion of study: (1) Human iRBCs perform equally as well as mouse blood to treat mild anemia in mice; and (2) Human iRBCs perform better than saline as a treatment for mild anemia in mice.

### Example 3

### Comparison to Other RBC Protocols

The iRBC methods described herein can be tested against other red cell formulas and methods; e.g., StemCell™ STEMdiff™ Erythroid Kit. Without wishing to be bound by theory, it is hypothesized that other RBC protocols promote differentiation but not maturation; i.e., those formulae achieve only early erythroblast transformation from iPS/ES cell sources (e.g., immature red cell progenitors, as found in the bone marrow), whereas the iRBC formula described herein results in complete maturation into the “adult” red cells, as found in blood circulation.

For example, the StemCell™ formula reports that 74% of cells will express the key red cell markers GlyA and CD71 by the end of the written protocol. The iRBC formula and methods described herein can result in 95-99% GlyA/CD71 positivity (see e.g., FIGS. 1C-1E, FIGS. 4B-4C, FIG. 6B, FIG. 7, FIG. 9B, FIGS. 11B-11C), showing that nearly all cells grown in the iRBC formula adopt a red cell identity, even sickle cells. Other protocols do not mention any statistics of red cell maturation that are used to determine that the cells are “adult” red cells, such as enucleation and globin expression. Based on the StemCell™ protocol, it is expected that the final product of StemCell™'s formula is a fairly heterogeneous culture of semi-differentiated and immature erythroblasts. While such cells could be used for erythroid research, these immature cells are not a clinically useful red cell product.

As such a side-by-side comparison of the iRBC formula described herein compared to other RBC formulas, such as StemCell™ STEMdiff™ Erythroid Kit is done to compare the quality of the resultant red cells. The iRBC formula results in a significantly higher percentage of mature RBCs, e.g., as measured using enucleation or globin expression, as compared to the StemCell™ formula.

1. A composition for inducing a red blood cell (RBC) comprising:
human AB plasma;
heparin;
insulin;
holo-transferrin;
a corticosteroid; and
polyvinyl alcohol (PVA).

2. The composition of claim 1, further comprising at least one growth factor.

3. The composition of claim 2, wherein the at least one growth factor is selected from the group consisting of IL-3, stem cell factor (SCF), and erythropoietin (EPO).

4. The composition of claim 1, wherein the corticosteroid is dexamethasone.

5. The composition of any of claims 1-4, further comprising Iscove's DMEM (IMDM).

6. The composition of any of claims 1-5, further comprising at least one antibiotic.

7. The composition of claim 6, wherein the at least one antibiotic is Penicillin and Streptomycin.

8. The composition of any of claims 1-7, wherein PVA is present at a 0.08% concentration.

9. The composition of any of claims 1-8, wherein the composition does not comprise bovine serum albumin (BSA).

10. The composition of any of claims 1-8, wherein each component of the composition is of pharmaceutical grade.

11. A composition for inducing a RBC comprising at least one of the components selected from the group consisting of: human AB plasma, an antibiotic, heparin, insulin, human holo-transferrin, a corticosteroid, and polyvinyl alcohol (PVA)

12. The composition of claim 11, further comprising IL-3, SCF, and EPO.

13. The composition of claim 11, further comprising SCF and EPO.

14. The composition of claim 11, further comprising EPO.

15. The composition of any of claims 11-14, wherein the composition does not comprise bovine serum albumin (BSA).

16. The composition of any of claims 11-15, wherein the PVA is present at a 0.08% concentration.

17. A method of inducing a red blood cell (RBC), the method comprising contacting a stem cell with the composition of any of claims 1-16 for a time sufficient to induce a RBC.

18. The method of claim 17, wherein the RBC is an enucleated RBC.

19. The method of claims 17 and 18, wherein the RBC expresses at least one cellular marker selected from GlyA (CD235a), Band 3, and CD71.

20. The method of claim 17, wherein the time sufficient is at least 18 days.

21. The method of claim 17, wherein the stem cell is an induced pluripotent stem cell (iPSC).

22. The method of claim 17, wherein the stem cell is a hematopoietic stem cell (HSC) or hematopoietic stem and progenitor cell (HSPC).

23. The method of any of claims 17-22, wherein contacting occurs on an ultra-low attachment culture dish.

24. The method of any of claims 17-23, wherein contacting occurs at 37° C. with at least 20% 0₂.

25. The method of any of claims 17-24, wherein the composition is replaced at least every 2 or 3 days.

26. A method of inducing a red blood cell (RBC), the method comprising
a) contacting a population of stem cells with the composition of claim 12;
b) contacting the population of a) with the composition of claim 13; and
c) contacting the population of b) with the composition of claim 14.

27. The method of any of claims 17-26, wherein contacting is in vitro or ex vivo.

28. The method of claim 27, wherein contacting is culturing.

29. A RBC produced by any of the methods of claims 11-28.

30. The RBC of claim 29, wherein the RBC is an enucleated RBC.

31. A composition comprising the RBC of claim 29 or 30, or population thereof

32. The composition of claim 31, further comprising a pharmaceutically acceptable carrier.

33. A pharmaceutical composition comprising the RBC of claim 29 or 30, or population thereof, and a pharmaceutically acceptable carrier.

34. The pharmaceutical composition of claim 33 for use in a blood transfusion in a subject.

35. A method of treating a subject in need of a blood transfusion, the method comprising administering a RBC of claim 29 or 30, or population thereof, or a composition of claims 31-32, or a pharmaceutical composition of claims 33-34 to a recipient subject in need thereof

36. The method of claim 35, wherein the subject in need thereof has a disease or disorder that inhibits proper RBC formation or production.

37. The method of claim 36, wherein the disease or disorder is selected from the group consisting of anemia, cancer, hemophilia, kidney disease, liver disease, severe microbial infection, sickle cell disease, and thrombocytopenia, a hemoglobinopathies, Diamond-Blackfan Anemia, iron deficiency, B12 deficiency, folate deficiency, dyserythropoietic anemias, hemolytic anemias, metabolic disorders, the porphyrias, autoimmune diseases.

38. The method of claim 35, further comprising, prior to administering, diagnosing a subject as having a disease or disorder that inhibits proper RBC formation or production.

39. The method of claim 35, further comprising, prior to administering, receiving the results of an assay that diagnoses a subject as having a disease or disorder that inhibits proper RBC formation or production.

40. The method of claim 35, wherein the subject in need thereof has a hemoglobin level below 10 g/dL, 9 g/dL, 8 g/dL, or below 7 g/dL.

41. The method of claim 35, further comprising, prior to administering, diagnosing a subject as having hemoglobin level below 10 g/dL, 9 g/dL, 8 g/dL, or below 7 g/dL.

42. The method of claim 35, further comprising, prior to administering, receiving the results of an assay that diagnoses a subject as having hemoglobin level below 10 g/dL, 9 g/dL, 8 g/dL, or below 7 g/dL.

43. A method of treating a disease or disorder that inhibits proper RBC formation or production, the method comprising administering a RBC of claim 29 or 30, or population thereof, or a composition of claims 31-32, or a pharmaceutical composition of claims 33-34 to a recipient subject diagnosed as having a disease or disorder that inhibits proper RBC formation or production.

44. The method of claim 43, further comprising, prior to administering, diagnosing a subject as having a disease or disorder that inhibits proper RBC formation or production.

45. The method of claim 43, further comprising, prior to administering, receiving the results of an assay that diagnoses a subject as having a disease or disorder that inhibits proper RBC formation or production.

46. The method of any of claims 35-45, wherein the RBC is autologous or allogenic to the subject.

47. A method of transfusing a population of autologous RBCs to a subject in need thereof, the method comprising
a) obtaining a stem cell source from a subject;
b) inducing a population of RBCs according to the method of any of claims 17-28; and
c) administering the induced RBC population of b) via transfusion to the subject.

48. A method of treating a disease or disorder that inhibits proper RBC formation or production, the method comprising
a) obtaining a stem cell source from a subject;
b) inducing a population of RBCs according to the method of any of claims 17-28; and
c) administering the induced RBC population of b) via transfusion to the subject diagnosed as having a disease or disorder that inhibits proper RBC formation or production.

49. The method of claim 47 or 48, further comprising, prior to administering, the step of genetically modifying the induced RBC of b).

50. The method of claim 47 or 48, further comprising, prior to inducing, the step of genetically modifying the stem cell source of a).

51. The method of claim 49 or 50, wherein genetically modifying corrects a disease gene carried by the subject.

52. A kit comprising any of the compositions of claims 1-16 and instructions for inducing a RBC using the composition.

53. A kit comprising the composition of claim 12, the composition of claim 13, and the composition of claim 14.

54. The kit of claim 52 or 53, further comprising a stem cell source.

55. A kit for inducing a RBC comprising:
a) a first composition comprising at least one of the components selected from the group consisting of: human AB plasma, an antibiotic, heparin, insulin, human holo-transferrin, a corticosteroid, and polyvinyl alcohol (PVA);
b) a second composition comprising IL-3, SCF, and EPO;
c) a third composition comprising SCF and EPO; and
d) a fourth composition comprising EPO and optionally human holo-transferrin.


SEQUENCE LISTING (20 sequences)

SEQ ID NO: 1 - DNA, 22 bp, Artificial Sequence
  synthetic oligonucleotide
gtgctgtccctgtatgcctctg

SEQ ID NO: 2 - DNA, 24 bp, Artificial Sequence
  synthetic oligonucleotide
gggagagcatagccctcgtagatg

SEQ ID NO: 3 - DNA, 19 bp, Artificial Sequence
  synthetic oligonucleotide
cgtgctggtctgtgtgctg

SEQ ID NO: 4 - DNA, 27 bp, Artificial Sequence
  synthetic oligonucleotide
cccccagtttagtagttggacttaggg

SEQ ID NO: 5 - DNA, 23 bp, Artificial Sequence
  synthetic oligonucleotide
ggacccagaggttctttgacagc

SEQ ID NO: 6 - DNA, 21 bp, Artificial Sequence
  synthetic oligonucleotide
ggaagtcagcaccttcttgcc

SEQ ID NO: 7 - DNA, 24 bp, Artificial Sequence
  synthetic oligonucleotide
gccagaacttcggcagtaaagaat

SEQ ID NO: 8 - DNA, 22 bp, Artificial Sequence
  synthetic oligonucleotide
cctgagagcttgctagtgattg

SEQ ID NO: 9 - DNA, 20 bp, Artificial Sequence
  synthetic oligonucleotide
tcctaagccactgcctgctg

SEQ ID NO: 10 - DNA, 24 bp, Artificial Sequence
  synthetic oligonucleotide
ttaacggtatttggaggtcagcac

SEQ ID NO: 11 - DNA, 29 bp, Homo sapiens
atggtgcatctgactcctgaggagaagtc

SEQ ID NO: 12 - DNA, 29 bp, Homo sapiens
atggtgcacctgactcctgtggagaagtc

SEQ ID NO: 13 - DNA, 29 bp, Homo sapiens
atggtgcacctgactcctgaggagaagtc

SEQ ID NO: 14 - protein, 10 aa, Homo sapiens
MVHLTPEEKS

SEQ ID NO: 15 - protein, 10 aa, Homo sapiens
MVHLTPVEKS

SEQ ID NO: 16 - DNA, 28 bp, Homo sapiens
ctgactccagaggagaagtctgccgtta

SEQ ID NO: 17 - DNA, 28 bp, Homo sapiens
ctgactcctgtggagaagtctgccgtta

SEQ ID NO: 18 - DNA, 28 bp, Homo sapiens
ctgactcctgaggagaagtctgccgtta

SEQ ID NO: 19 - protein, 9 aa, Homo sapiens
LTPEEKSAV

SEQ ID NO: 20 - protein, 9 aa, Homo sapiens
LTPVEKSAV